(19) United States
(12) Patent Application Publication
(10) Pub. No.: US 2004/0002056 A1
(43) Pub. Date: Jan. 1, 2004

(54) Methods of screening for bioactive agents using cells transformed with self-inactivating viral vectors

(76) Inventors: James B. Lorens, Portola Valley, CA (US); David A. Ferrick, El Macero, CA (US)

Correspondence Address: LAHIVE & COCKFIELD, 28 STATE STREET, BOSTON, MA, 02109, US

(21) Appl. No.: 10/151,750

(22) Filed: May 15, 2002

Related U.S. Application Data
(63) Continuation-in-part of application No. 10/133,973, filed on Apr. 24, 2002.
(63) Continuation-in-part of application No. 09/710,058, filed on Nov. 10, 2000.
(63) Continuation-in-part of application No. 09/966,976, filed on Sep. 27, 2001.
Application No. 09/966,976 is a continuation of application No. 09/963,206, filed on Sep. 25, 2001.
Application No. 09/966,976 is a continuation of application No. 09/963,247, filed on Sep. 25, 2001.
Application No. 09/963,247 is a division of application No. 09/076,624, filed on May 12, 1998.
Application No. 09/963,247 is a division of application No. 09/712,821, filed on Nov. 13, 2000.
(60) Provisional application No. 60/290,287, filed on May 10, 2001.
(60) Provisional application No. 60/164,592, filed on Nov. 10, 1999.
(60) Provisional application No. 60/165,189, filed on Nov. 12, 1999.

(51) Int. Cl. C12Q 1/70; C12Q 1/68; C12N 15/861

(52) U.S. Cl. 435/5; 435/6; 435/456

(57) ABSTRACT

The invention relates to cells transformed with self-inactivating retroviral vectors and their use in methods of screening for candidate bioactive agents that produce an altered phenotype in the cells.

### CROSS REFERENCE TO RELATED APPLICATIONS

This application is a continuation-in-part application of U.S. Ser. No. 09/076,624, filed May 12, 1998, application U.S. Ser. No. 09/712,821 filed Nov. 13, 2000, and application U.S. Ser. No. 10/133,973 filed Apr. 24, 2002. The content of each of these applications is hereby incorporated by reference in their entirety.

### FIELD OF THE INVENTION

The invention relates to methods and compositions useful in screening for candidate agents having biological activity. Specifically, the present invention is drawn to methods for identifying biologically active molecules using cells transformed with self-inactivating (SIN) viral vectors expressing fusion nucleic acids.

### BACKGROUND OF THE INVENTION

Stable cell lines expressing a gene of interest provide significant advantages in studying biological processes and in screens for biologically and pharmacologically active agents. Once isolated, a transformed cell line provides a stable source of gene of interest. There is low variability in expression between cells and all cells express the gene. Uniformly and consistent expression permits facile identification of a cell phenotype when the cells are subjected to a variety of manipulations, for example when exposed to ligands of cell surface receptors. In addition, expressing a gene of interest allows for manipulating the phenotype of cells, which are then useful in identifying agents that alter or change the induced cellular phenotype. These properties afforded by stably transformed cell lines enable large scale screens for candidate agents having biological and pharmacological activity.

Stable cell lines expressing a fusion nucleic acid may be obtained by transient transfection of cells with an expression vector expressing a selectable marker, such as a drug resistance gene. Stable expression relies on non-homologous integration into the chromosome, which is generally random in nature. Difficulties in transient transfections include the need to optimize the transfection process for each cell type being analyzed due to inherent differences in DNA uptake efficiencies. More importantly, generating stable cell lines requires a lengthy process for selecting and cloning the stable lines.

Stable cell lines expressing genes of interest can also be generated based on homologous recombination mechanisms. Generally described as a “knock-in” or “knock-out” process, the DNA used for recombination have DNA sequences substantially similar to the target sequences on the host chromosome. Recombination between the substantially similar sequences by strand invasions leads to insertion of the nucleic acid vector into the host chromosome. Since homologous recombination is limited by the presence of homologous sequences within the host chromosome, insertion of multiple constructs are difficult. Moreover, as the homologous sequences are frequently directed to coding regions of known genes, the integrated nucleic acid is potentially subject to regulatory influence by cellular sequences that normally control expression of the coding region. This may interfere with the activity of promoters present on the integrated fusion nucleic acid. Moreover, homologous recombination is inefficient since a majority of cells fail to stably integrate the nucleic acid of interest.

Stable integration of nucleic acids may also rely on site-specific recombination mediated by recombinases. In these processes, specific recombinases catalyze a reciprocal double-stranded DNA exchange between two DNA segments by recognizing specific sequences present on both partners of the exchange. Specific recombinases are found in both prokaryotes and eukaryotes. In prokaryotes, the λ-integrase acts to insert λ phage into bacterial chromosomes. Similarly transposon integrases, such a γδ resolvase, function to allow integration of transposons into specific sequences within the bacterial genome. Promiscuity of the integration depends on the sequence elements recognized by the resolvase or integrase. Both the resolvase and integrase constitute members of the “tyrosine recombinases” which include flp recombinase of yeast and cre-lox recombinase of P1 bacteriophage.

An analogous system for site specific recombination in eukaryotic cells are the integrases involved in integration of retroviruses. Specificity of integration derives from recognition of specific sequences located at the ends of the linear viral DNA intermediates. The integration is essentially random since insertions occur with high promiscuity, although biases (i.e., hot spots) for particular chromosomal sites are known. After integration, the provirus stably resides in the host chromosome. Consequently, by engineering retroviruses to accommodate non-viral nucleic acids, retroviruses serve as efficient vectors for gene transfer and for creation of cell lines stably transformed with exogenous nucleic acids.

Common retroviral vectors, however, have several drawbacks. First, the presence of viral promoters at the 5′ long terminal repeats (LTR) may result in mobilization or rescue of an integrated provirus by endogenous retroviruses or upon infection with retroviral vectors that express viral proteins. In addition, the expressed viral RNA can recombine with retroviral RNAs, for example during propagation of the vector, to reconstitute replication competent retroviruses.

Additional problems associated with retroviral vectors are that the promoter elements at the 3′ LTR region can potentially activate or influence expression of nearby endogenous genes on the host chromosome, thereby producing undesirable phenotypes in cells harboring the provirus. Moreover, the promoter at the 5′ LTR of the provirus may interfere with internal promoters used to express non-viral nucleic acids within the retroviral vector, which may result in inconsistent expression of the non-viral nucleic acid.

Self-inactivating (SIN) retroviral vectors reduce these problems by removing or inactivating the promoter elements at the 3′ LTR, which results in elimination of promoter elements from both 5′ and 3′ LTR of the integrated viral DNA. Accordingly, the present invention uses the advantages of cells transformed with SIN vectors for use in screening for candidate agents with biological and pharmacological activity.

### SUMMARY OF THE INVENTION

In accordance with the objects outline above, the present invention provides methods of screening for candidate bioactive agents capable of producing an altered phenotype in a transformed cell. The method comprises combining a candidate agent and a transformed cell comprising a SIN vector, or a plurality of SIN vectors, and screening the cells for an altered phenotype.

In one aspect, the SIN vector comprises a promoter operably linked to a gene of interest. In another aspect, the SIN vector comprises a promoter operably linked to a first gene of interest, a separation sequence, and a second gene of interest. When separation sequences are used, the separation sequence may be a protease recognition sequence, an IRES element, or a Type 2A sequence. The gene of interest may comprise a reporter gene, a selection gene, a nucleic acid encoding a dominant effect protein, or combinations thereof. Various reporter/selection genes or combinations of reporter/selection genes may be used for identifying cells displaying a particular phenotype.

The present invention further relates to methods of screening for candidate agents capable of regulating promoter activity. These screens comprise providing a cell or a plurality of cells transformed with SIN vectors, which comprise fusion nucleic acids containing a promoter of interest, combining the cells with at least one candidate agent, and screening the cells for an altered phenotype. The promoter of interest is operably linked to a fusion nucleic acid comprising a gene of interest, or a fusion nucleic acid comprising a first gene of interest, a separation sequence, and a second gene of interest. Detecting expression of the gene(s) of interest permits identification of candidate agents that directly or indirectly regulate promoter activity. When the promoter of interest is inducible, inducing agent is used to activate the promoter. This provides a method of screening for candidate agents that affect inducing processes, such as signal transduction pathways.

In another preferred embodiment, the SIN vectors are used to express candidate agents in the transformed cells. Candidate agents expressed from the SIN vectors include cDNAs, cDNA fragments, genomic DNA fragments, and random nucleic acids, which may or may not encode peptides.

In the present invention, the transformed cells may comprise a plurality of SIN vectors. In one aspect, the plurality of SIN vectors in a cell express different genes of interest. Thus, in one preferred embodiment, at least one SIN vector expresses a candidate agent while at least one other SIN vector expresses gene(s) of interest used for detecting an altered phenotype. Alternatively, at least one of the SIN vector expresses a gene of interest which regulates the promoter of another SIN vector in the cell, thus allowing regulated expression of other SIN vectors. In this way, expression of candidate agents may be regulated during the screening process.

The methods of the present invention further comprise isolating from the plurality of cells a cell with an altered phenotype and identifying the candidate agent producing the altered phenotype. Accordingly, the present invention provides methods of identifying biologically and pharmacologically active agents and the cognate target molecules affected by the candidate agents.

### BRIEF DESCRIPTION OF THE FIGURES

FIG. 1 shows the nucleotide sequence of the a long terminal repeat (LTR) of Moloney Murine Leukemia Virus (MMLV) (upper sequence) and a self-inactivating deletion in a SIN LTR (lower sequence). The SIN deletion removes the duplicated enhancer elements (present from about nucleotide positions −342 to about −174) and the CAAT box (at about nucleotide position -80) in the U3 segment. A TATA box present at −20 nucleotide position is intact in the SIN LTR, which results in a low basal level of viral promoter activity. The R region begins at nucleotide position 0 and contains the poly A site, AATAAA, at about nucleotide position 50.

FIG. 2 shows a SIN expression vector used to generate promoter reporter cell lines. The retroviral construct comprises a CMV promoter operably linked to the 5′ end of a retroviral genome (see Naviux et al. (1996) “The pCL Vector System: Rapid Production of Helper Free, High Titre, Recombinant Viruses,” J. Virol. 70: 5701-05) and an extended packaging signal y for packaging of viral RNA into virions. The 3′ end of the viral genome comprises a SIN deletion in the U3 region, as described in FIG. 1. Within the viral genome, a promoter is operably linked to a selectable marker (e.g., a reporter gene) via an intron, which results in efficient expression of the selectable marker. Introns may be from a natural intron associated with the selectable marker gene or introns of other genes, such a β-globin intron (see Lorens et al. (2000) Virology 272: 7-15). A polyadenylation signal, pA, or a polyA tract enhances translation of the transcribed selectable marker gene. To produce viral particles, the retroviral plasmid construct is transfected into a packaging cell line (e.g., 293 cell-based Phoenix A amphotropic cell line). Transcription from the CMV promoter produces RNAs, which are packaged into virions. Following infection of a host cell and integration of the viral construct into a host chromosome, the deleted U3 segment in the 3′ LTR is duplicated at the 5′ LTR, resulting in loss of viral promoter/enhancer activity.

FIG. 3A depicts a retroviral construct used to generate cell lines that serve as screening cells for agents modulating the IgE ε promoter. The retroviral construct comprises anε promoter fragment containing various enhancer elements (e.g., C/EBP) operably linked via an intron, for example a β-globin intron, to a GFP reporter gene. Deletion within the U3 region generates the SIN feature of the retroviral construct. FIG. 3B shows FACS analysis of B cell line CA46 transduced with the promoter reporter fusion nucleic acid. Upon transduction of CA46 cells with retroviruses, 14.3% of non-IL4 induced cells express detectable GFP while 19.6% of IL-4 induced cells express the reporter molecule. Cell line D5 isolated from the transduced CA46 cell population displays little or no GFP expression in the absence of IL-4 induction. Upon treatment with IL-4, 99.7% of the cells have detectable GFP fluorescence, thus showing that the ε promoter in the D5 clone is highly responsive to signal transduction events mediated by IL-4.

FIG. 4 shows two retroviral promoter reporter constructs used for generating cells lines useful in screening for agents affecting IgH promoter activity. Construct p129 and p132 is based on a SIN vector backbone similar to that described in FIG. 2. p129 and p132 has an intronic enhancer element, Eμ, linked to a IgH promoter, V_{H}. The promoter drives expression of a fusion nucleic acid comprising a first gene of interest comprising HBEGF, a separation sequence of FMDV 2A, and a second gene of interest comprising a GFP gene fused to a PEST sequence (dsGFP; Clontech, Palo Alto, Calif.). A bovine growth hormone polyadenylation signal (BGH pA) and an intron from the β-globin gene allow efficient expression of the encoded proteins. Construct p132 is same as p129 except that a 3′ enhancer element, 3′αE, is inserted downstream of the polyadenylation signal.

FIG. 5 shows composition of a cell used in a screen for candidate agents that affect signal transduction pathways involved in regulating IgH promoter activity. The cell comprises a SIN vector based promoter reporter, p132 (described in FIG. 4) and a SIN vector comprising a tetracycline regulated promoter (TRE) operably linked to a blue fluorescent protein gene, which is fused to nucleic acids encoding random peptides (BFP-RP). The cell line also contains a retroviral construct that expresses a tetracyclin regulatable tranactivator, tTA, which regulates synthesis of the candidate agent, BFP-RP.

Stimulation of the B cell receptor (BCR) with anti-IgM F(ab)2 antibodies activates signal transduction events leading to activation of IgH promoter activity, and thus synthesis of HBEGF and dsGFP. Selecting for cells expressing no or low GFP levels in the absence of tetracyclin analog, doxycylin, identifies cells expressing candidate peptides that inhibit activation of the IgH promoter. Treatment with diptheria toxin provides a more stringent selection for cells with low IgH promoter activity. Following isolation low GFP expressing cells, treatment with doxycyclin should result in increased GFP expression after restimulation of the BCR receptor if the expressed candidate peptide inhibits signaling pathways involved in activation of the IgH promoter.

### DETAILED DESCRIPTION OF THE INVENTION

The availability of cell lines stably transformed with exogenous nucleic acids provides a useful platform for examining biological processes and for drug screening. The self-inactivating (SIN) retroviral vectors allow for generating stably transformed cell lines but without the attendant problems associated with vectors having active viral promoters and enhancers. Accordingly, the present invention relates to cells transformed with retroviral SIN vectors.

By “retroviral vectors” herein is meant vectors used to introduce into a host the fusion nucleic acids of the present invention in the form of a RNA viral particle, as is generally outlined in PCT US 97/01019 and PCT US 97/01048, both of which are incorporated by reference. Various retroviral vectors are known, including vectors based on the murine stem cell virus (MSCV) (see Hawley, R. G. et al. (1994) Gene Ther. 1: 136-38), modified MFG virus (Riviere, I. et al. (1995) Genetics 92: 6733-37), pBABE (see PCT US97/01019), and pCRU5 (Naviaus, R. K. et al. (1996) J. Virol. 70: 5701-05); all references are hereby expressly incorporated by reference. In addition, particularly well suited retroviral transfection systems for generating retroviral vectors are described in Mann et al., supra; Pear, W. S. et al. (1993) Pro. Natl. Acad. Sci. USA 90: 8392-96; Kitamura, T. et al. (1995) Proc. Natl. Acad. Sci. USA 92: 9146-50; Kinsella, T. M. et al. (1996) Hum. Gene Ther. 7: 1405-13; Hofmann, A. et al. (1996) Proc. Natl. Acad. Sci. USA 93: 5185-90; Choate, K. A. et al. (1996) Hum. Gene Ther. 7: 2247-53; WO 94/19478; PCT US 97/01019, and references cited therein, all of which are incorporated by reference.

In a preferred embodiment, the retroviral vectors are self-inactivating retroviral vectors or SIN vectors. By “self-inactivating” or “SIN” or grammatical equivalents herein is meant retroviral vectors in which the viral promoter elements are rendered ineffective or inactive (see Yu, S.-F. et al. (1986) Proc. Natl. Acad. Sci. USA 83: 3094-84). These promoter and enhancer elements are present in the 3′ long terminal repeat (3′ LTR), which is composed of segments designated as U3 and R (see John M. Coffin, Retroviridae: The Viruses and Their Replication, in Virology, Vol. 2, 1767-1847 (Bernard M. Fields et al. eds.) (3rd ed. 1996). The integrated retroviral genome, called the provirus, is bounded by two LTRs and is transcribed from the 5′ LTR to the 3′ LTR. The viral promoters and enhancers reside generally in the U3 region of the 3′ LTR, but the 3′ LTR region is duplicated at the 5′ LTR during viral integration. Promoter elements situated at the 5′ LTR direct expression of virally encoded genes and generate the RNA copies that are packaged into viral particles.

The self-inactivating feature of SIN vectors arises from the mechanism of viral replication and integration (see Coffin, supra). Following entry of the retrovirus into a cell, a tRNA molecule binds to the primer binding region (PB) at the 5′ end of the viral RNA. Extension of the tRNA primer by reverse transcriptase results in a tRNA linked to a DNA segment containing the U5 and R sequences present at the 5′ end of the viral RNA. RNase activity of reverse transcriptase acts on the viral RNA strand of the DNAIRNA hybrid, thus releasing the elongated tRNA, which then hybridizes to complementary R sequences present on the 3′ end of the viral genome. Elongation by reverse transcriptase results in synthesis of a DNA copy of the viral genome (minus strand DNA) and degradation of the RNA strand by RNase. A short RNA sequence designated the PP sequence, which is resistant to RNase action, remains hybridized to the newly synthesized DNA strand—generally at a region immediately preceding the U3 region at the 3′ end of the viral genome—and acts as a primer for replication of the complementary strand (plus strand DNA). Extension of this PP primer results in replication of sequences comprising U3, R, U5, and PB segments, which eventually become the 5′ LTR of the integrated virus. Subsequently, the PB region of the extended primer hybridizes to the complementary PB region present on the 3′ end of the minus strand DNA, and subsequent extension of this hybrid results in synthesis of a double strand DNA intermediate in which the 5′ and 3′ LTR contain the U3, R, and U5 segments. Following replication and transport into the nucleus, the viral double stranded DNA integrates into the host chromosome via the attachment sites (att) present near the ends of the LTRs, to generate the integrated provirus.

Since the mechanism of viral replication results in duplication of the promoter elements at the 3′ LTR to the 5′ LTR of the integrated virus, inactivating or replacing the viral promoter results in inactivating or replacing the promoter normally present in the proviral 5′ LTR. This feature describes the self-inactivating nature of these retroviral vectors. Inactivation of the 5′ LTR promoter reduces expression of the proviral nucleic acid from the 5′ LTR and reduces the potential deleterious effects arising from influences on cellular genes by the viral promoter present on the 3′ LTR of the integrated virus.

Accordingly, the SIN vectors of the present invention comprise fusion nucleic acids in which the viral promoter elements, as generally defined below, are rendered inactive or ineffective. By “ineffective” is meant a promoter whose transcriptional activity is reduced by about 80% as compared to promoter activity of the intact viral promoter/enhancer or other measurable promoter activities in the cell. Preferred are reductions in promoter activities of about 90%, with most preferred being inactivation of the viral promoter/enhancer as compared to a cellular promoter or intact viral promoter. By “inactivation” or grammatical equivalents herein is meant that transcription directed by viral sequences in not detected by the assays described below or is about 1% or lower than that of an identifiable promoter activity, such as a constitutively active promoter.

In the present invention, promoter activity is assessed relative to identifiable promoter activities, such as comparisons to constitutively expressed cellular transcripts, for example glyceraldehyde 3′ phosphate dehydrogenase (G3PHD). Another measure of promoter activity is by use of fusion nucleic acids comprising a heterologous promoter, for example SV40 early promoter or CMV promoter operably linked to a reporter or selection gene (Yu, S-F, et al., supra). In one preferred embodiment, the heterologous promoter construct is introduced into cells via retroviral vectors to generate stably integrated fusion nucleic acids expressing the reporter/selection gene. Direct comparisons of promoter activities are also possible by replacing the viral genes, such as gag, env and pol with a reporter or selection gene. This arrangement positions the 5′ LTR of the provirus to directly regulate expression of the reporter or selection gene, thus allowing comparisons of promoter activity between intact and altered (i.e., inactive) viral promoters. In addition, the retroviral fusion nucleic acid further comprises an independent promoter (e.g., CMV promoter) directing expression of a second reporter or selection gene, which provides a basis for selecting transformed cells harboring the fusion construct used to assess promoter activity.

Promoter activity is measured by methods well known in the art, including Northern hybridization, primer extension, or detecting expression of a reporter or selection gene (e.g., by growing cells in presence of selection agent). Alternatively, promoter activity is measurable by a viral rescue assay. If the viral promoters on the 5′ LTR of the provirus are active, the expressed viral RNAs are packaged when the transformed cells are transfected with fusion nucleic acids that provide viral proteins necessary for packaging the viral RNAs expressed from the provirus (see for example, Miyoshi, H. et al. (1998) J. Virol. 72: 8150-57). Following release of the packaged viruses from the cell, the cellular media is examined for the number of infectious viral particles retaining the reporter gene by infecting a population of cells and assaying for reporter gene expression.

Ineffectiveness or inactivation of the promoter is measured in the cell in which the vector is expressed. Thus, where alterations of the viral promoter renders the promoter active in particular cell types while inactive in others, the retroviral vector is a SIN vector with respect to the cell types in which the altered promoter and/or enhancer is ineffective or inactive. For example, deletion of cell specific viral promoter/enhancer elements can reduce or eliminate transcriptional activity of viral promoter in those particular cells where the promoter/enhancer is active while retaining transcriptional activity in other cells.

Altering the viral promoter/enhancer to render it ineffective or inactive to produce SIN vectors is accomplished by various methods well known to those skilled in the art. In one aspect, enhancer and promoter elements are deleted. Deletions at the 3′ LTR is generally at the U3 region of the 3′ LTR. For example, a 299 bp deletion of the U3 of MoMuLV removes the 72 bp repeat enhancer elements and the canonical “CAAT” sequence, essentially inactivating the viral promoter (see Yu, supra). Since complete elimination of U3 region may negatively affect polyadenylation signals, deletions may be restricted to certain enhancer and promoter elements to maintain high titre production of retroviral vectors. Thus, deletions may be directed specifically to certain enhancer or promoter elements or combinations thereof. Alternatively, the deletions comprise a series of deletions progressively removing longer segments of the suspected promoter and/or enhancer region to inactivate viral promoters without seriously compromising virus production or proviral expression (Iwakuma, T. et al. (1999) Virology 261: 120-32). The promoter elements, including enhancers, are well known for various retroviruses (see Coffin, supra).

In another aspect, mutagenesis is used to render the viral promoter and/or enhancers ineffective or inactive (U.S. Pat. No. 5,672,510). Various mutagenesis techniques are well known, including oligonucleotide directed mutagenesis, error prone replication, and chemical mutagenesis. Mutagenesis by insertions of nucleic acids, for example by linker scanning mutagenesis or other insertional mutagenesis, are also useful for inactivating promoters and enhancers (see Steffy, K.R. (1991) J. Virol. 65: 6454-60; Haapa, S. (1999) Nucleic Acids Res. 27: 2777-84). As with deletions, mutagenesis may be directed towards the whole 3′ LTR segment comprising the viral promoter element, or restricted to specific promoter and/or enhancer elements and combinations thereof.

In another preferred embodiment, the viral promoter elements are replaced or substituted with other nucleic acids. In one aspect, the replacement or substitution is with promoter/enhancer sequences from other organisms or cells, thus creating a vector in which the promoters/enhancers are active in particular cell types while inactive in other types of cells. These types of constructs allow for efficient propagation of the virus in one cell type while retaining the SIN features in another cell type (Ferrari, G. et al. (1995) Hum. Gene Ther. 6: 733-42).

Alternatively, in a preferred embodiment, the replacement or substitution sequence is an inducible promoter, for example a tetracyclin inducible promoter, tetP, to generate conditional SIN vectors. In the absence of induction (e.g., presence of tetracyclin analog, doxycycline), the virally associated inducible promoter is inactive, thus generating a SIN phenotype as described herein. The ability to manipulate the SIN phenotype provides several advantages, including (1) efficient propagation of retrovirus, (2) retention of SIN phenotype for wide variety of cell types, and (3) inducible expression of provirual nucleic acids.

In the present invention, SIN vectors are generally made so as to preserve efficient expression of the fusion nucleic acid of the provirus. These include the polyadenlylation signals needed for efficient expression of viral transcripts and viral propagation, integrations sites (i.e., aft L) required for insertion of the viral DNA intermediate into the host chromosome, and preservation of mRNA splicing signals when needed for postranscriptional processing of the transcript. In some cases, the efficiency of viral replication may be enhanced by incorporation nonviral elements, such as non-viral polyadenylation signals or poly A tracts, etc.

Since retroviral vectors allow for delivery of various nucleic acids, the SIN vectors of the present invention further comprise fusion nucleic acids useful for introducing and expressing other nucleic acids, including nucleic acids expressing genes of interest. By “fusion nucleic acid” herein is meant a plurality of nucleic acid components that are joined together, either directly or indirectly. As will be appreciated by those in the art, in some embodiments the sequences described herein may be DNA, for example when extrachromosomal plasmids are used, or RNA when retroviral vectors are used. In some embodiments, the sequences are directly linked together without any linking sequences while in other embodiments linkers such as restriction endonuclease cloning sites, linkers encoding flexible amino acids, such as glycine or serine linkers such as known in the art, are used, as further discussed below.

As one aspect of the SIN vectors is to express nucleic acids, the fusion nucleic acids of the present invention further comprises a promoter. By “promoter” as defined herein is meant nucleic acid sequences capable of initiating transcription of the fusion nucleic acid or portions thereof. Promoter may be constitutive wherein the transcription level is constant and unaffected by modulators of promoter activity. Promoter may also be inducible in that promoter activity is capable of being increased or a decreased, for example as measured by the presence or quantitation of transcripts or of translation products (see Walther, W. et al. (1996) J. Mol. Med. 74: 379-92; Mills, A. A. (2001) Genes Dev. 15: 1461-67; and White, J.H. (1997) Adv. Pharmacol. 40: 339-67). Promoter may also be cell specific wherein the promoter is active only in particular cell types. In this sense, promoter as defined herein includes sequences required for initiating and regulating the level of transcription and transcription in specific cell types. Thus, included within the definition of promoter are enhancer elements which act to regulate transcription generally or transcription in specific cell types. Furthermore, the promoters of the present invention include within derivatives or mutant promoters, and hybrid promoters formed by combining elements of more than one promoter. Preferred promoters for expression in mammalian cells are CMV promoters and hybrid tetracycline inducible promoters, such as tetP.

Generally, the transcriptional regulatory nucleic acid sequences are operably linked to the nucleic acids to be expressed. Nucleic acid is “operably linked” when it is placed into a functional relationship with another nucleic acid sequence. In this context, operably linked means that the transcriptional and other regulatory nucleic acids are positioned relative to a coding sequence in such a manner that transcription is initiated. Generally, this will mean that the promoter and transcriptional initiation or start sequences are positioned 5′ to the coding region. The transcriptional regulatory nucleic acid selected will be appropriate to the host cell used, as will be appreciated by those in the art. Numerous types of appropriate expression vectors, and suitable regulatory sequences, are known in the art for a variety of host cells. In addition, the fusion nucleic acids of the present invention comprise nucleic acid sequences necessary for efficient translation of expressed fusion nucleic acid such as translation initiation sequences, polyadenylation signals, mRNA splicing signals, all of which are well known in the art.

The SIN vectors of the present invention are used to express fusion nucleic acids in a cell transformed with the SIN vector. The expressed fusion nucleic acid may or may not code for a protein. In one preferred embodiment, the expressed nucleic acids do not code for a protein but is capable of having a biological effect on the cell. In one aspect, the nucleic acid may be an antisense nucleic acid directed toward a complementary target nucleic acid. As is well known in the art, antisense nucleic acids find use in suppressing or affecting expression of various genes of pathogenic organisms or expression of cellular genes. These include suppression of oncogenes to affect the proliferative properties of transformed cells (Martiat, P. et al. (1993) Blood 81: 502-09; Daniel, R. (1995) Oncogene 10: 1607-14; Niemeyer, C. C. (1998) Cell Death Differ. 5: 440-49), modulate cell cycle (Skotz, M. et al. (1995) Cancer Res. 55: 5493-98), inhibit proteins involved in cardiovascular disease states (Wang, H. (1999) Circ. Res. 85: 614-22) and inhibit viral pathogenesis (Lo, K. M. et al. (1992) Virology 190: 176-83; Chatterjee, S. et al (1992) Science 258: 1485-88).

In another aspect, the expressed nucleic acids are nucleic acids capable of catalyzing cleavage of target nucleic acids in a sequence specific manner, preferably in the form of ribozymes. Ribozymes include, among others, hammerhead ribozymes, hairpin ribozymes, and hepatitis delta virus ribozymes (Tuschl, T. (1995) Curr. Opin. Struct. Biol. 5: 296-302; Usman, N. (1996) Curr Opin Struct Biol 6: 527-33; Chowrira, B. M. et al. (1991) Biochemistry 30: 8518-22; and Perrotta A. T. et al. (1992) Biochemistry 3: 16-21). As with antisense nucleic acids, nucleic acids catalyzing cleavage of target nucleic acids may be directed to a variety of expressed nucleic acids, including those from pathogenic organisms or cellular genes (see Jackson, W. H. et al. (1998) Biochem. Biophys. Res. Commun. 245: 81-84).

In another aspect, the expressed nucleic acids are double stranded RNA capable of inducing RNA interference or RNAi (Bosher, J. M. et al. (2000) Nat. Cell Biol. 2: E31-36). Introducing double stranded RNA can trigger specific degradation of homologous RNA sequences, generally within the region of identity of the dsRNA (Zamore, P. D. et. al. (1997) Cell 101: 25-33). This provides a basis for silencing expression of genes, thus permitting a method for altering the phenotype of cells. The dsRNA may comprise synthetic RNA made either by known chemical synthetic methods or by in vitro transcription of nucleic acid templates carrying promoters (e.g., T7 or SP6 promoters). Alternatively, the dsRNAs are expressed in vivo using SIN vectors, preferably by expression of palindromic fusion nucleic acids, that allow facile formation of dsRNA in the form of a hairpin when expressed in the cell. The double strand regions of the hairpin RNA are generally about 10-500 basepairs or more, preferably 15-200 basepairs, and most preferably 20-100 basepairs.

Since the expressed nucleic acids produce an identifiable phenotype in the cell (i.e., a dominant phenotype), these cells provide a basis for identifying candidate agents, such as random nucleic acids or random peptides, which alter the cellular phenotype arising from the expressed nucleic acid. For example, if the expressed nucleic acid affects a signal transduction pathway, candidate agents that inhibit or activate the pathway may be identified in a screen.

In another preferred embodiment, the SIN vectors are used to express fusion nucleic acids comprising a gene of interest, or as explained below, a plurality of genes of interest, such as a first and a second gene of interest. By “gene of interest” herein is meant any nucleic acid sequence capable of encoding a “protein of interest” or a “protein,” as defined below. However, in some embodiments, the “gene of interest” encompasses a regulatory element that does not encode a protein. These elements may include, but are not limited to, promoter/enhancer elements, chromatin organizing sequences, ribosome binding sequences, mRNA splicing sequences, etc.

In one preferred embodiment, the gene of interest is a reporter gene. By “reporter gene” or “selection gene” or grammatical equivalents herein is meant a gene that by its presence in a cell (e.g., upon expression) allows the cell to be distinguished from a cell that does not contain the reporter gene. Reporter genes can be classified into several different types, including detection genes, survival genes, death genes, cell cycle genes, cellular biosensors, proteins producing a dominant cellular phenotype, and conditional gene products. In the present invention, expression of the protein product causes the effect distinguishing between cells expressing the reporter gene and those that do not. As is more fully outlined below, additional components, such as substrates, ligands, etc., may be additionally added to allow selection or sorting on the basis of the reporter gene.

In a preferred embodiment, the gene of interest is a reporter gene. The reporter gene encodes a protein that can be used as a direct label, for example a detection gene for sorting the cells or for cell enrichment by FACS. In this embodiment, the protein product of the reporter gene itself can serve to distinguish cells that are expressing the reporter gene. Suitable reporter genes include those encoding green fluorescent protein (GFP, Chalfie, M. et al. (1994) Science 263: 802-05; and EGFP, Clontech—Genbank Accession Number U55762), blue fluorescent protein (BFP, Quantum Biotechnologies, Inc. 1801 de Maisonneuve Blvd. West, 8th Floor, Montreal (Quebec) Canada H3H 1J9; Stauber, R. H. (1998) Biotechniques 24: 462-71; and Heim, R. et al. (1996) Curr. Biol. 6: 178-82), enhanced yellow fluorescent protein (EYFP, Clontech Laboratories, Inc., 1020 East Meadow Circle, Palo Alto, Calif. 94303), Anemonia majano fluorescent protein (amFP486, Matz, M. V. (1999) Nat. Biotech. 17: 969-73), Zoanthus fluorescent proteins (zFP506 and zFP538; Matz, supra), Discosoma fluorescent protein (dsFP483, drFP583; Matz, supra), Clavularia fluorescent protein (cFP484; Matz, supra); luciferase (for example, firefly luciferase, Kennedy, H. J. et al. (1999) J. Biol. Chem. 274: 13281-91; Renilla reniformis luciferase, Lorenz, W. W. (1996) J Biolumin. Chemilumin. 11: 31-37; Renilla muelleri luciferase, U.S. Pat. No. 6,232,107); β-galactosidase (Nolan, G. et al. (1988) Proc. Natl. Acad. Sci. USA 85: 2603-07); β-glucouronidase (Jefferson, R. A. et al. (1987) EMBO J. 6: 3901-07; Gallager, S., GUS Protocols: Using the GUS Gene as a reporter of gene expression, Academic Press, Inc.(1992)); and secreted form of human placental alkaline phosphatase, SEAP (Cullen, B. R. et al. (1992) Methods Enzymol. 216: 362-68). In a preferred embodiment, the codons of the reporter genes are optimized for expression within a particular organism, especially mammals, and particularly for humans (see Zolotukhin, S. et al. (1996) J. Virol. 70: 4646-54; U.S. Pat. No. 5,968,750; U.S. Pat. No. 6,020,192; all of which are expressly incorporated by reference).

In a preferred embodiment, the codons of the reporter genes are optimized for expression within a particular organism, especially mammals, and particularly preferred for human cell expression (see Zolotukhin, S. et al. (1996) J. Virol. 70: 4646-54; U.S. Pat. No. 5,968,750; U.S. Pat. No. 6,020,192; U.S. S. No. 60/290,287, all of which are expressly incorporate by reference).

In another embodiment, the reporter gene encodes a protein that will bind a label that can be used as the basis of the cell enrichment (sorting); that is, the reporter gene serves as an indirect label or detection gene. In this embodiment, the reporter gene preferably encodes a cell-surface protein. For example, the reporter gene may be any cell-surface protein not normally expressed on the surface of the cell, such that secondary binding agents serve to distinguish cells that contain the reporter gene from those that do not. Alternatively, albeit non-preferably, reporters comprising normally expressed cell-surface proteins could be used, and differences between cells containing the reporter construct and those without could be determined. Thus, secondary binding agents bind to the reporter protein. These secondary binding agents are preferably labeled, for example with fluors, and can be antibodies, haptens, etc. For example, fluorescently labeled antibodies to the reporter gene can be used as the label. Similarly, membrane-tethered streptavidin could serve as a reporter gene, and fluorescently-labeled biotin could be used as the label, i.e., the secondary binding agent. Alternatively, the secondary binding agents need not be labeled as long as the secondary binding agent can be used to distinguish the cells containing the construct; for example, the secondary binding agents may be used in a column, and the cells passed through, such that expression of the reporter gene results in the cell being bound to the column, and a lack of the reporter gene (i.e., inhibition), results in the cells not being retained on the column. Other suitable reporter proteins/secondary labels include, but are not limited to, antigens and antibodies, enzymes and substrates (or inhibitors), etc.

In a preferred embodiment, the reporter gene is a survival gene that serves to provide a nucleic acid iL5 (or encode a protein) without which the cell cannot survive, such as drug resistance genes. In this embodiment, expressing the survival gene allows selection of cells expressing the fusion nucleic acid by identifying cells that survive, for example in presence of a selection drug. Examples of drug resistance genes include, but are not limited to, puromycin resistance gene (puromycin-N-acetyl-transferase; de la Luna, S. et al. (1992) Methods Enzymol. 216: 376-85), G418 neomycin resistance gene, hygromycin resistance gene (hph), and blasticidine resistance genes (bsr, brs, and BSD; Pere-Gonzalez, et al.(1990) Gene, 86: 129-34; Izumi, M. et al. (1991) Exp. Cell Res. 197: 229-33; Itaya, M. et al. (1990) J. Biochem. 107: 799-801; and Kimura, M. et al. (1994) Mol. Gen. Genet. 242: 121-29). In addition, generally applicable survival genes are the family of ATP-binding cassette transporters, including multiple drug resistance gene (MDR1) (see Kane, S. E. et. al. (1988) Mol. Cell. Biol. 8: 3316-21 and Choi, K. H. et al. (1988) Cell 53: 519-29), multi-drug resistance associated proteins (MRP) (Bera, T. K. et al. (2001) Mol. Med. 7: 509-16), and breast cancer associated protein (BCRP or MXR) (Tan, B. et al. (2000) Curr. Opin. Oncol. 12: 450-58). When expressed in cells, these selectable genes can confer resistance to a variety of toxic reagents, especially anti-cancer drugs (i.e., methotrexate, colchicine, tamoxifen, mitoxanthrone, doxorubicin, etc.). As will be appreciated by those skilled in the art, the choice of the selection/survival gene will depend on the host cell type used.

In a preferred embodiment, the reporter gene encodes a death gene that causes the cells to die when expressed. Death genes fall into two basic categories: death genes that encode death proteins requiring a death ligand to kill the cells, and death genes that encode death proteins that kill cells as a result of high expression within the cell and do not require the addition of any death ligand. Preferred are cell death mechanisms that requires a two-step process: the expression of the death gene and induction of the death phenotype with a signal or ligand such that the cells may be grown expressing the death gene, and then induced to die. A number of death genes/ligand pairs are known, including, but not limited to, the Fas receptor and Fas ligand (Schneider, P. et al. (1997) J. Biol. Chem. 272: 18827-33; Gonzalez-Cuadrado, S. et al. (1997) Kidney Int. 51: 1739-46; and Muruve, D. A. et al. (1997) Hum. Gene Ther. 8: 955-63); p450 and cyclophosphamide (Chen, L. et al. (1997) Cancer Res. 57: 4830-37); thymidine kinase and gangcylovir (Stone, R. (1992) Science 256: 1513); diptheria toxin and heparin-binding epidermal growth factor-like growth factor (HBEGF; see WO 01/34806, hereby incorporated by reference); and tumor necrosis factor (TNF) receptor and TNF. Alternatively, the death gene need not require a ligand, and death results from high expression of the gene, for example, the overexpression of a number of programmed cell death (PCD) proteins known to cause cell death, including, but not limited to, caspases, bax, TRADD, FADD, SCK, MEK, etc.

In a preferred embodiment, death genes also include toxins that cause cell death, or impair cell survival or cell function when expressed by a cell. These toxins generally do not require addition of a ligand to produce toxicity. An example of a suitable toxin is campylobacter toxin CDT (Lara-Tejero, M. (2000) Science, 290: 354-57). Expression of CdtB subunit, which has homology to nucleases, causes cell cycle arrest and ultimately cell death. Another toxin, the diptheria toxin (and similar Pseudomonas exotoxin), functions by ADP ribosylating the ef-2 (elongation factor 2) molecule in the cell and preventing translation. Expression of the diptheria toxin A subunit induces cell death in cells expressing the toxin fragment. Other useful toxins include cholera toxin and pertussis toxin (catalytic subunit-A ADP ribosylates the G protein regulating adenylate cyclase), pierisin from cabbage butterflys (induces apoptosis in mammalian cells; Watanabe, M. (1999) Proc. Natl. Acad. Sci. USA 96: 10608-13), phospholipase snake venom toxins (Diaz, C. et al. (2001) Arch. Biochem. Biophys. 391: 56-64), ribosome inactivating toxins (e.g., ricin A chain, Gluck, A. et al. (1992) J. Mol. Biol. 226: 411-24;and nigrin, Munoz, R. et al. (2001) Cancer Lett. 167: 163-69), and pore forming toxins (e.g., hemolysin and leukocidin). When the target cells are neuronal cells, neuronal specific toxins may be used to inhibit specific neuronal functions. These include bacterial toxins such as botulinum toxin and tetanus toxin, which are proteases that act on synaptic vesicle associated proteins (e.g., synaptobrevin) to prevent neurotransmitter release (see Binz, T. et al. (1994) J. Biol. Chem. 269: 9153-58; Lacy, D. B. et al. (1998) Curr. Opin. Struct. Biol. 8: 778-84). Another preferred embodiment of a reporter molecule is a cell cycle gene; that is, a gene that causes alterations in the cell cycle. For example, Cdk interacting protein p21 (Harper, J. W. et al. (1993) Cell 75: 805-16), which inhibits cyclin dependent kinases, does not cause cell death but causes cell-cycle arrest. Consequently, expressing p21 allows selecting for regulators of promoter activity or regulators of p21 activity based on detecting cells that grow out much more quickly due to low p21 activity, either through inhibiting promoter activity or inactivation of p21 protein activity. As will be appreciated by those in the art, it is also possible to configure the system to select cells based on their inability to grow out due to increased p21 activity. Similar mitotic inhibitors include p27, p57, p16, p15, p18 and p19, p19 ARF (human homolog p14 ARF). Other cell cycle proteins useful for altering cell cycle include cyclins (Cln), cyclin dependent kinases (Cdk), cell cycle checkpoint proteins (i.e. Rad17, p53), Cks1 p9, Cdc phosphatases (i.e Cdc 25) etc.

In yet another preferred embodiment, the gene of interest encodes a cellular biosensor. By a cellular biosensor herein is meant a gene product that when expressed within a cell can provide information about a particular cellular state. Biosensor proteins allow rapid determination of changing cellular conditions, for example Ca⁺² levels in the cell, pH within cellular organelles, and membrane potentials (see Miesenbock, G. et al. (1998) Nature 394: 192-95). An example of an intracellular biosensor is Aequorin, which emits light upon binding to Ca⁺² ions. The intensity of light emitted depends on the Ca⁺² concentration, thus allowing measurement of transient calcium concentrations within the cell. When directed to particular cellular organelles by fusion partners, as more fully described below, the light emitted by Aequorin provides information about Ca⁺² concentrations within the particular organelle. Other intracellular biosensors are chimeric GFP molecules engineered for fluorescence resonance energy transfer (FRET) upon binding of an analyte, such as Ca⁺² (Miyawaki, A. et al. (1997) Nature 388: 882-87; Miyakawa, A. et al. (1997) Mol. Cell. Biol. 8: 2659-76). For example, Camelot consists of blue or cyan mutant of GFP, calmodulin, CaM binding domain of myosin light chain kinase, and a green or yellow GFP. Upon binding of Ca⁺² by the CaM domain, FRET occurs between the two GFPs because of a structural change in the chimera. Thus, FRET intensity is dependent on the Ca⁺² levels within the cell or organelle (Kerr, R. et al. Neuron (2000) 26: 583-94). Other examples of intracellular biosensors include sensors for detecting changes in cell membrane potential (Siegel, M. et al. (1997) Neuron 19: 735-41; Sakai, R. (2001) Eur. J. Neurosci. 13: 2314-18), monitoring exocytosis (Miesenbrock, G. et al. (1997) Proc. Natl. Acad. Sci. USA 94: 3402-07), and measuring intracellular/organellar ATP concentrations via luciferase protein (Kennedy, H. J. et al. (1999) J. Biol. Chem. 274: 13281-91). These biosensors find use in monitoring the effects of various cellular effectors, for example pharmacological agents that modulate ion channel activity, neurotransmitter release, ion fluxes within the cell, and changes in ATP metabolism.

Other intracellular biosensors comprise detectable gene products with sequences that are responsive to changes in intracellular signals. These sequences include peptide sequences acting as substrates for protein kinases, peptides with binding regions for second messengers, and protein interaction sequences sensitive to intracellular signaling events (see for example, U.S. Pat. No. 5,958,713 and U.S. Pat. No. 5,925,558). For example, a fusion protein construct comprising a GFP and a protein kinase recognition site allows measuring intracellular protein kinase activity by measuring changes in GFP fluorescence arising from phosphorylation of the fusion construct. Alternatively, the GFP is fused to a protein interaction domain whose interaction with cellular components are altered by cellular signaling events. For example, it is well known that inositol-triphosphate (InsP3) induces release of Ca⁺² from intracellular stores into the cytoplasm, which results in activation of a kinases responsible for regulating various cellular responses. The precursor to InsP3 is phosphatidyl-inositol4,5-bisphosphate (PtdInsP₂), which is localized in the plasma membrane and cleaved by phospholipase C (PLC) following activation of an appropriate receptor. Many signaling enzymes are sequestered in the plasma membrane through pleckstrin homology domains that bind specifically to PtdInsP₂. Following cleavage of PtdInsP₂, the signaling proteins translocate from the plasma membrane into the cytosol where they activate various cellular pathways. Thus, a reporter molecule such as GFP fused to a pleckstrin domain will act as a intracellular sensor for phospholipase C activation (see Haugh, J. M. et al. (2000) J. Cell. Biol. 15: 1269-80; Jacobs, A. R. et al. (2001) J. Biol. Chem. 276: 40795-802; and Wang, D. S. et al. (1996) Biochem. Biophys. Res. Commun. 225: 420-26). Other similar constructs are useful for monitoring activation of other signaling cascades and applicable as assays in screens for candidate agents that inhibit or activate particular signaling pathways.

Since protein interaction domains, such as the described pleckstrin homology domain, are important mediators of cellular responses and biochemical processes, other preferred genes of interest are proteins containing protein-interaction domains. By “protein-interaction domain” herein is meant a polypeptide region that interacts with other biomolecules, including other proteins, nucleic acids, lipids, etc. These protein domains frequently act to provide regions that induce formation of specific multiprotein complexes for recruiting and confining proteins to appropriate cellular locations or affect specificity of interaction with targets ligands, such as protein kinases and their substrates. Thus, many of these protein domains are found in signaling proteins. Protein-interaction domains comprise modules or micro-domains ranging about 20-150 amino acids that can be expressed in isolation and bind to their physiological partners. Many different interaction domains are known, most of which fall into classes related by sequence or ligand binding properties. Accordingly, the genes of interest comprising interaction domains may comprise proteins that are members of these classes of protein domains and their relevant binding partners. These domains include, among others, SH2 domains (src homology domain 2), SH3 domain (src homology domain 3), PTB domain (phosphotyrosine binding domain), FHA domain (forkedhead associated domain), WW domain, 14-3-3 domain, pleckstrin homology domain, C1 domain, C2 domain, FYVE domain (i.e., Fab-1, YGLO23, Vps27, and EEA1), death domain, death effector domain, caspase recruitment domain, Bcl-2 homology domain, bromo domain, chromatin organization modifier domain, F box domain, hect domain, ring domain (e.g., Zn⁺² finger binding domain), PDZ domain (PSD-95, discs large, and zona occludens domain), sterile a motif domain, ankyrin domain, arm domain (armadillo repeat motif), WD 40 domain and EF-hand (calretinin), PUB domain (Suzuki T. et al. (2001) Biochem. Biophys. Res. Commun. 287:1083-87), nucleotide binding domain, Y Box binding domain, H.G. domain, all of which are well known in the art. Since protein interactions domains are pervasive in cellular signal transduction cascades and other cellular processes, such as cell cycle regulation and protein degradation, expression of single proteins or multiple proteins with interaction domains acting in specific signaling or regulatory pathway may provide a basis for inactivating, activating, or modulating such pathways in normal and diseased cells. In another aspect, the preferred embodiments comprise binding partners of these interactions domains, which are well known to those skilled in the art or are identifiable by well known methods (e.g., yeast two hybrid technique, co-precipitation of immune complexes, etc.).

Included within the protein-interaction domains are transcriptional activation domains capable of activating transcription when fused to an appropriate DNA binding domain. Transcriptional activation domains are well known in the art. These include activator domains from GAL4 (amino acids 1-147; Fields, S. et al. (1989) Nature 340: 245-46; Gill, G. et al. (1990) Proc. Natl. Acad. Sci. USA 87: 2127-31), GCN4 (Hope, I. A. et al. (1986) Cell 46: 885-94), ARD1 (Thukral, S. K. et al. (1989) Mol. Cell. Biol. 9: 2360-69), human estrogen receptor (Kumar, V. et al. (1987) Cell 51: 941-51), VP16 (Triezenberg, S. J. et al. (1988) Genes Dev. 2: 718-29), Sp1 (Courey, A.J. (1988) Cell 55: 887-98), AP-2 (Williams, T. et al. (1991) Genes Dev. 5: 670-82), and NF-kB p65 subunit and related Rel proteins (Moore, P. A. et al. (1993) Mol. Cell. Biol. 13: 1666-74). DNA binding domains include, among others, leucine zipper domain, homeo box domain, Zn⁺² finger domain, paired domain, LIM domain, ETS domain, and T Box domain.

Since the genes of interest may comprise DNA binding domains and transcriptional activation domains, other genes of interest useful for expression in the present invention are transcription factors. Preferred transcription factors are those producing a cellular phenotype when expressed within a particular cell type. Transcription factors as defined herein include both transcriptional activator or inhibitors. As not all cells will respond to expression of a particular transcription factor, those skilled in the art can choose appropriate cell strains in which expression of a transcription factor results in dominant or altered phenotypes as described below.

In another aspect, the transcription factor regulates expression of a different promoter of interest on a retroviral vector that does not encode the transcription factor. This arrangement requires introducing a plurality or multiple retroviral vectors into a single cell, as described below, one of which expresses the transcription factor regulating the different promoter of interest. Expression of the transcription factor is inducible or the transcription factor itself is an inducible transcription factor, thus allowing further regulation of the different promoter of interest.

In an alternative embodiment, the transcription factor encoded by the gene of interest regulates the promoter on the retroviral vector encoding the transcription factor. These constructs are autoregulatory for expression of the retroviral vector (Hofmann, A. (1996) Proc. Natl. Acad. Sci. USA 93: 5185-90). Accordingly, if the transcription factor inhibits promoter activity on the retroviral vector, continued synthesis of transcription factor restricts expression of the viral fusion nucleic acids. On the other hand, if the transcription factor activates transcription, synthesis is elevated because of continued synthesis of the transcriptional activator. Consequently, by use of separation sequences, as described below, to express a plurality of genes of interest, one of which encodes the transcription factor, the retroviral vector autoregulates expression of the genes of interest. To enhance autoregulation, the transcription factor is an inducible transcription factor, for example a tetracycline or steroid inducible transcription factor (e.g., RU-486 or ecdysone inducible; see White J H (1997) Adv. Pharmacol. 40: 339-67). Incorporation of an inducible transcription factor in a retroviral vector as a single autoregulatory cassette eliminates the need for additional vectors for regulating the promoter activity. Moreover, this system results in rapid, uniform expression of the gene(s) of interest.

In another preferred embodiment, the gene of interest encodes a protein whose expression has a dominant effect on the cell (i.e., produces an altered cellular phenotype). By “dominant effect” herein is meant that the protein or peptide produces an effect upon the cell in which it is expressed and is detected by the methods described below. The dominant effect may act directly on the cell to produce the phenotype or act indirectly on a second molecule, which leads to a specific phenotype. Dominant effect is produced by introducing small molecule effectors, expressing a single protein, or by expressing multiple proteins acting in combination (i.e., synergistically on a cellular pathway or multisubunit protein effectors). As is well known in the art, expression of a variety of genes of interest may produce a dominant effect. Expressed proteins may be mutant proteins that are constitutive for a catalytic activity (Segouffin-Cariou, C. et al. (2000) J. Biol. Chem. 275: 3568-76; Luo et al. (1997) Mol. Cell. Biol. 17: 1562-71) or are inactive forms that sequester or inhibit activity of normal binding partners (Bossu, P. (2000) Oncogene, 19: 2147-54; Mochizuki, H. (2001) Proc. Natl Acad. Sci. USA 98: 10918-23). The inactive forms as defined herein include expression of small modular protein-interaction regions or other domains that bind to binding partners in the cell (see for example, Gilchrist, A. et al. (1999) J. Biol. Chem. 274: 6610-16). Dominant effects are also produced by overexpression of normal cellular proteins, expression of proteins not normally expressed in a particular cell type, or expression of normally functioning proteins in cells lacking functional proteins due to mutations or deletions (Takihara, Y. et al. (2000) Carcinogenesis 21: 2073-77; Kaplan, J.B. (1994) Oncol. Res. 6: 611-15). Random peptides or biased random peptides introduced into cells can also produce dominant effects. An exemplary effect of a dominant effect by a peptide is random peptides which bind to Src SH3 domain resulting in increased Src activity due to the peptides' antagonistic effect on negative regulation of Src (see Sparks, A. B. et al. (1994) J Biol Chem. 269: 23853-56).

As defined herein, dominant effect is not restricted to the effect of the protein on the cell expressing the protein. A dominant effect may be on a cell contacting the expressing cell or by secretion of the protein encoded by the gene of interest into the cellular medium. Proteins with dominant effect on other cells are conveniently directed to the plasma membrane or secretion by incorporating appropriate secretion and/or membrane localization signals. These membrane bound or secreted dominant effector proteins may comprise cytokines and chemokines, growth factors, toxins (e.g., neurotoxins), extracellular proteases (e.g., metalloproteases), cell surface receptor ligands (e.g., sevenless type receptor ligands), adhesion proteins (e.g., L1, cadherins, integrins, laminin), etc.

In an alternative embodiment, the gene of interest encodes a conditional gene product. By “conditional gene” product herein is meant a gene product whose activity is only apparent under certain conditions, for example at particular ranges of temperature. Other factors that conditionally affect activity of a protein include, but are not limited to, ion concentration, pH, and light (see Hager, A. (1996) Planta 198: 294-99; Pavelka J. (2001) Bioelectromagnetics 22: 371-83). A conditional gene product produces a specific cellular phenotype under a restrictive condition. In contrast, the conditional gene product does not produce a specific phenotype under permissive conditions. Methods for making or isolating conditional gene products are well known (see for example White, D. W. et al. (1993) J. Virol. 67:6876-81; Parini, M.C. (1999) Chem. Biol. 6: 679-87).

As is appreciated by those skilled in the art, conditional gene products are useful in examining genes that are detrimental to a cell's survival or in examining cellular biochemical and regulatory pathways in which the gene product functions. For those gene products that affect cell survival, use of conditional gene products allows survival of the cells under permissive conditions, but results in lethality or detriment at the restrictive condition. This feature allows screens at the restrictive condition for candidate agents, such as proteins and small molecules, which may directly or indirectly suppress the effect of conditional gene product, but permit maintenance and growth of cells under permissive conditions. In addition, conditional gene products are also useful in screens for regulators of cell physiology when the conditional gene product is a participant in a cellular regulatory pathway. At the restrictive condition, the conditional gene product ceases to function or becomes activated, resulting in an altered cell phenotype due to dysregulation of the regulatory pathway. Candidate agents are then screened for their ability to activate or inhibit downstream pathways to bypass the disrupted regulatory point. Conditional gene products are well known in the art and include, among others, proteins such dynamin involved in endocytic pathway (Damke, H. et al. (1995) Methods Enzymol. 257: 209-20), p53 involved in tumor suppression (Pochampally, R. et al. (2000) Biochem. Biophys. Res. Comm. 279: 1001-10 and Buckbinder, L. et al. (1994) Proc. Natl. Acad. Sci. USA 91: 10640-44), Vac1 involved in vesicle sorting, proteins involved in viral pathogenesis (SV40 Large T Antigen; Robinson C. C. (1980). J Virol. 35: 246-48) and gene products involved in regulating the cell cycle, such as ubiquitin conjugating enzyme CDC 34 (Ellison, K. S. et al. (1991) J. Biol. Chem. 266: 24116-20).

Since candidate bioactive agents comprising candidate nucleic acids, as described below, are capable of encoding proteins, candidate nucleic acids are encompassed within the genes of interest described above. Thus, genes of interest expressed by retroviral vectors, including the SIN vectors described herein, may comprise candidate bioactive agents in the form of libraries of cDNAs, genomic DNAs, candidate nucleic acids encoding peptides (random or biased random), as further defined below.

As indicated above, the SIN vectors of the present invention also find use in expressing a plurality of genes of interest. By “plurality” herein is meant more than one gene of interest. Thus, the SIN vector comprising the fusion nucleic acid may comprise a “gene of interest” or a “first gene of interest” and additional genes of interest such as a “second gene of interest.” Use of separation sequences incorporated into the fusion nucleic acids, as described below, allow for synthesis of separate protein products encoded by the genes of interest; alternatively, polyproteins may be made as is known in the art, either through the use of linkers, as defined herein, or through direct fusions.

In one embodiment, the first and second gene of interest encode the same gene. These constructs allow increased expression of the encoded protein product since two copies of the same gene of interest are expressed in a single transcriptional event. Synthesizing high levels of encoded protein is desirable when needed to produce a cellular phenotype (e.g., dominant or altered phenotype) through maintaining elevated cellular levels of an effector protein, or in industrial applications where maximizing production of a gene of interest is needed to increase efficiency and lower manufacturing costs. Similarly, for example when screening for promoter regulators, signal amplification may be accomplished using two identical reporter genes such as GFP.

In a more preferred embodiment, the first gene of interest is non-identical to the second gene of interest. Thus, the first gene of interest and the second gene of interest may have different nucleic acid sequences, which may manifest itself as differences in amino acid sequence, protein size, protein activities, or protein localization. Since expressing multiple gene products have utility in many different biological, diagnostic, and medical applications, the present invention envisions numerous combinations of a first gene of interest and second gene of interest. Those skilled in the art can choose the combinations most relevant to their needs. For example, two different reporter genes can be used, such as distinguishable GFPs.

Accordingly, in one preferred embodiment, at least one of the genes of interest of the fusion nucleic acid encodes a reporter gene. The presence of a separation sequence allows the synthesis of separate proteins of interest and reporter proteins, thus allowing detecting expression of the gene of interest by monitoring coexpression of the reporter protein. Producing separate reporter proteins and proteins of interest obviate any detrimental effect that might arise from fusing a reporter protein to the protein of interest. Additionally, expressing separate reporter proteins and proteins of interest allows targeting of individual proteins to distinct cellular locations. In some situations, the reporter protein is also an indicator of cellular phenotype, which provides a means for detecting the cell expressing the fusion nucleic acid, but also provides information about the physiological state of the cell.

In another aspect, at least one of the genes of interest is a selection gene. Expression of the gene of interest and a selection gene permits selecting for cells expressing both the gene of interest and the selection gene, for example, a neomycin resistance. The presence of separation sequence produces separate protein products of the gene of interest and selection gene, which is important for the reasons described above. If the selection gene is either survival or death gene, their expression in cells is useful in screening for agents that counteract or regulate the action of survival genes.

In another aspect, at least one of the genes of interest encodes a protein producing a dominant effect on a cell. As described above, dominant effect is produced in a variety of ways. The protein may be overexpressed natural proteins or expressed mutants, variants, or analogs of the natural protein.

Classes of proteins producing a dominant effect include signal transduction proteins, protein-interaction domains, cell cycle regulatory proteins, or transcription factors whose expression produces a detectable phenotype in a cell. The expressed protein is active in producing the dominant effect or is active conditionally, requiring a restrictive condition to produce the cellular phenotype. Fusion nucleic acids where at least one of the gene of interest encodes a protein having a dominant effect provides a basis for screening for candidate agents inhibiting or enhancing the dominant effect.

In another preferred embodiment, at least one of the gene of interest comprises a candidate agent. The candidate agents may be cDNA, fragment of cDNA, genomic DNA fragment, or candidate nucleic acids encoding random or biased random peptides. Expression of fusion nucleic acids where the first gene of interest is a candidate agent and a second gene of interest is a reporter gene allows selection of cells expressing the candidate agent. Alternatively, if the second gene of interest encodes a protein producing a dominant effect, expression of a variety of candidate agents—as a first gene of interest—will permit screening of candidate agents acting as effectors or regulators of the dominantly active protein. By “effector” herein is meant inhibition, activation, or modulation of the cellular phenotype produced by the dominant effect protein. For example, the dominantly acting protein may have a tyrosine kinase activity which activates or inhibits signaling cascades to produce a detectable cellular phenotype. Expression of candidate agents can identify candidate agents acting as kinase inhibitors that suppress the phenotype generated by the protein encoded by the second gene of interest.

As the present invention allows for various combinations of first gene of interest and second gene of interest, one preferred combination is a first and second gene of interest encoding two different reporter/selection proteins. These constructs provide two different basis for detecting a cell expressing the fusion nucleic acid. For example, the first gene of interest may be a GFP and the second gene of interest a β-galactosidase, which permits increased discrimination of cells expressing the fusion nucleic acid by detecting both GFP and β-galactosidase activities. Alternatively, another combination comprises a first gene of interest comprising a reporter gene and a second gene of interest comprising a selection gene. This allows selection for cells expressing fusion nucleic acid based on expression of the selection gene, such as a drug resistance gene (e.g., puromycin) or a death gene (e.g., HGEGF plus diptheria toxin), as well as expression of the reporter construct.

Another preferred combination is where the first gene of interest encodes a first survival gene and the second gene of interest encodes a second survival gene. Thus, one embodiment of the fusion nucleic acid comprises a first gene of interest encoding a first multidrug resistance gene (e.g., MDR-1) and a second gene of interest encoding a second multidrug resistance gene (e.g., MRP). Both MDR-1 and MRP are ATP cassetted transporters implicated in development of cellular tolerance to toxic drugs, especially anti-cancer agents. Expression of these multiple multidrug resistance transporters in cancerous cells can limit the effectiveness of chemotherapy. Accordingly, expressing several different multidrug resistance genes allows screening for candidate agents or combination of candidate agents (drug cocktails) effective in inhibiting multiple drug resistance genes.

In another embodiment, a preferred combination is a first gene of interest encoding a first death gene and the second gene of interest encodes a second death gene. Particularly preferred are death genes involved in a particular death pathway, such as caspase proteases involved in apoptotic pathways and apoptosis related gene Apaf-1 (Cecconi, F. (1999) Cell Death Differ. 6: 1087-98). In some embodiments, expression of one death gene may be insufficient to produce a cell death phenotype, and thus require expression of multiple death related genes. Accordingly, expression of multiple death gene are used to produce a cell death phenotype, for example by expression of Fas and Fas binding protein FADD (Chang, H. Y. et al. (1999) Proc. Natl. Acad. Sci. USA 96: 1252-56).

In another embodiment, the first gene of interest comprises a first biosensor and the second gene of interest comprises second biosensor. Use of different biosensors permit monitoring of more than one intracellular event. For example, the first gene of interest is an Aequorin Ca⁺² sensor protein while the second is a distinguishable pleckstrin homology-GFP fusion protein, such as pleckstrin-EGFP. This allows simultaneous monitoring of intracellular Ca⁺² and receptor mediated phospholipase C signaling activation, which may be useful in identifying cellular elements involved in regulating the IP3 signaling pathway and screening of candidate agents that act on specific steps of the IP3 signaling process.

Similarly, another preferred combination is a first gene of interest encoding a first dominant effector and the second gene of interest encodes a second dominant effector. Particularly preferred are dominant effectors acting synergistically or acting in combination to produce a cellular phenotype. One example is coexpression of GAP and Ras to produce transformed phenotype in cells (see Clark G. J. et al. (1997) J. Biol. Chem. 272: 1677-81). The GAP protein appears to contribute to Ras transforming activity by activating the GTPase activity of Ras. By expressing both GAP and Ras in the same cell, the oncogenic potential by the Ras pathway is elevated.

When expressing a plurality of genes of interest, there is no particular order of the genes of interest on the fusion nucleic acid. One embodiment may have a first gene of interest upstream of a second gene of interest. Another embodiment may have the second gene of interest upstream and the first gene of interest downstream. By “upstream” and “downstream” herein is meant the proximity to the point of transcription initiation, which is generally localized 5′ to the coding sequence of the fusion nucleic acid. Thus, in a preferred embodiment, the upstream gene of interest is more proximal to the transcription initiation site than the downstream gene of interest.

As will be appreciated by those skilled in the art, the positioning of the first gene of interest relative to the second gene of interest is determined by the person skilled in the art. Factors to consider include the need for detecting expression of a gene of interest or optimizing the levels of synthesis of the protein of interest. In the embodiments described above, where at least one of the genes of interest is a reporter gene, the reporter gene may be placed downstream of the gene of interest so that expression of the reporter gene will be a faithful indication of expression of the gene of interest. This will depend on the types of separation sites chosen by the person skilled in the art. When protease cleavage or Type 2A separation sequences are incorporated into the fusion nucleic acid, a reporter gene situated downstream of the gene of interest will generally provide direct information on expression of the upstream gene of interest. In the case of IRES sequences, however, detecting expression of the reporter to monitor expression of the upstream gene of interest is less direct since separate translation initiations occur for the first and second genes of interest, generally resulting in lower amount of the second protein being made. In some cases, the ratio of expression of first and second proteins can be as high as 10:1.

The order of the gene of interest on the fusion nucleic acid and the choice of separation sequence is also important when the relative amounts of first and second gene products of interest are at issue. For example, use of IRES sequences may result in lower amounts of downstream gene product as compared to upstream gene product because of differing translation initiation rates. Relative levels of translation initiation is easily determined by comparing expression of upstream gene of interest versus downstream gene of interest. Where controlling expression levels are important, the person skilled in the art will order the gene product needed at higher levels upstream of the downstream gene product when IRES separation sequences are used. Alternatively, multiple copies of IRES sequences are adaptable to increase expression of the downstream gene of interest. On the other hand, use of protease or Type 2A separation sequences will lessen the need for ordering the genes of interest on the fusion nucleic acid since these separation sequences tend to produce equal levels of upstream and downstream gene product.

When the SIN vectors expresse separate protein products encoded by the genes of interest, the fusion nucleic acids further comprises separation sequences. By a “separation sequence” or “separation site” or grammatical equivalents as used herein is meant a sequence that results in protein products not linked by a peptide bond. Separation may occur at the RNA or protein level. By being separate does not preclude the possibility that the protein products of the first gene of interest and the second gene of interest interact either non-covalently or covalently following their synthesis. Thus, the separate protein products may interact through hydrophobic domains, protein-interaction domains, common bound ligands, or through formation of disulfide linkages between the proteins.

Various types of separation sequences may be employed. In one embodiment, the separation sequence encodes a recognition site for a protease. A protease recognizing the site cleaves the translated protein product into two or more proteins. Preferred protease cleavage sites and cognate proteases include, but are not limited to, prosequences of retroviral proteases including human immunodeficiency virus protease, and sequences recognized and cleaved by trypsin (EP 578472), Takasuga, A. et al. (1992) J. Biochem. 112: 652-57), proteases encoded by Picornaviruses (Ryan, M. D. et al. (1997) J. Gen. Virol. 78: 699-723), factor Xₐ (Gardella, T. J. et al. (1990) J. Biol. Chem. 265: 15854-59; WO 9006370), collagenase (J03280893; WO 9006370; Tajima, S. et al. (1991) J. Ferment. Bioeng. 72: 362), clostripain (EP 578472), subtilisin (including mutant H64A subtilisin, Forsberg, G. et al. (1991) J. Protein Chem. 10: 517-26), chymosin, yeast KEX2 protease (Bourbonnais, Y. et al. (1988) J. Bio. Chem. 263: 15342-47), thrombin (Forsberg et al., suPra; Abath, F. G. et al. (1991) BioTechniques 10: 178), Staphylococcus aureus V8 protease or similar endoproteinase-Glu-C to cleave after Glu residues (EP 578472; Ishizaki, J. et al. (1992) Appl. Microbiol. Biotechnol. 36: 483-86), cleavage by Nla proteainase of tobacco etch virus (Parks, T. D. et al. (1994) Anal. Biochem. 216: 413-17), endoproteinase-Lys-C (U.S. Pat. No. 4,414,332) and endoproteinase-Asp-N, Neisseria type 2 IgA protease (Pohlner, J. et al. (1992) Biotechnology 10: 799-804), soluble yeast endoproteinase yscF (EP 467839), chymotrypsin (Altman, J. D. et al. (1991) Protein Eng. 4: 593-600), enteropeptidase (WO 9006370), lysostaphin, a polyglycine specific endoproteinase (EP 316748), the family of caspases (e.g., caspase 1, caspase 2, capase 3, etc.), and metalloproteases.

The present invention also contemplates protease recognition sites identified from a genomic DNA, cDNA, or random nucleic acid libraries (see for example, O'Boyle, D. R. et al. (1997) Virology 236: 338-47). For example, the fusion nucleic acids of the present invention may comprise a separation site which is a randomizing region for the display of candidate protease recognition sites. The first and second gene of interest encode reporters molecules useful for detecting protease activity, such as GFP molecules capable of undergoing FRET via linkage through a candidate recognition site (see Mitra, R. D. et al. (1996) Gene;173: 13-7). Proteases are expressed or introduced into cells expressing these fusion nucleic acids. Random peptide sequences acting as substrates for the particular protease result in separate GFP proteins, which is manifested as loss of FRET signal. By identifying classes of recognition sites, optimal or novel protease recognition sequences may be determined.

In addition to their use in producing separate proteins of interest, the protease cleavage sites and the cognate proteases are also useful in screening for candidate agents that enhance or inhibit protease activity. Since many proteases are crucial to pathogenesis of organisms or cellular regulation, for example the HIV or caspase proteases, the ability to express reporter or selection proteins linked by a protease cleavage site allows screens for therapeutic agents directed against a particular protease acting on the recognition site.

Another embodiment of separation sequences are internal ribosome entry sites (IRES). By “internal ribosome entry sites”, “internal ribosome binding sites”, or “IRES elements”, or grammatical equivalents herein is meant sequences that allow CAP independent initiation of translation (Kim, D. G. et al. (1992) Mol. Cell. Biol. 12: 3636-43; McBratney, S. et al. (1993) Curr. Opin. Cell Biol. 5: 961-65).

IRES sequences appear to act by recruiting 40S ribosomal subunit to the mRNA in the absence of translation initiation factors required for normal CAP dependent translation initiation. IRES sequences are heterogenous in nucleotide sequence, RNA structure, and factor requirements for ribosome binding. They are frequently located on the untranslated leader regions of RNA viruses, such as the Picornaviruses. The viral sequences range from about 450-500 nucleotides in length, although IRES sequences may also be shorter or longer (Adam, M. A. et al. (1991) J. Virol. 65: 4985-90; Borman, A. M. et al. (1997) Nucleic Acids Res. 25: 925-32; Hellen, C. U. et al. (1995) Curr. Top. Microbiol. Immunol. 203: 31-63; and Mountford, P. S. et al. (1995) Trends Genet. 11: 179-84). Embodiments of viral IRES separation sites are the Type I IRES sequences present in entero- and rhinoviruses and Type II sequences of cardioviruses and apthoviruses (e.g., encephalomyocarditis virus; see Elroy-Stein, O. et al. (1989) Proc. Natl. Acad. Sci. USA 86: 6126-30; Alexander, L. et al. (1994) Proc. Natl. Acad. Sci. USA 91: 1406-10). Other viral IRES sequences are found in hepatitis A viruses (Brown, E. A. et al. (1994) J. Virol. 68: 1066-74), avian reticuloendotheleliosis virus (Lopez-Lastra, M. et al. (1997) Hum. Gene Ther. 8: 1855-65), Moloney murine leukemia virus (Vagner, S. et al. (1995) J. Biol. Chem. 270: 20376-83), short IRES segments of hepatitis C virus (Urabe, M. et al. (1997) Gene 200: 157-62), and DNA viruses (e.g., Karposi's sarcoma-associated virus, Bieleski, L. et al. (2001) J. Virol. 75:1864-69).

Additionally, preferred embodiments of IRES sequences are non-viral IRES elements found in a variety of organisms including yeast, insects, worms, plants, birds, and mammals. Like the viral IRES sequences, cellular IRES sequences are heterogeneous in sequence and secondary structure. Cellular IRES sequences, however, may comprise shorter nucleic acid sequences as compared to viral IRES elements (Oh, S. K. et al. (1992) Genes Dev. 6: 1643-53; Chappell, S. A. et al. (2000) 97: 1536-41). Specific non-viral IRES elements include, but are not limited to, sequences that direct translation initiation of immunoglobulin heavy chain binding protein, transcription factors, protein kinases, protein phosphatases, eIF4G (see Johannes, G. et al. (1999) Proc. Natl. Acad. Sci. USA 96: 13118-23; Johannes, G. et al. (1998) RNA 4: 1500-13), vascular endothelial growth factor (Huez, I. et al. (1989) Mol. Cell. Biol. 18: 6178-90), c-myc (Stoneley, M. et al. (2000) Nucleic Acids Res. 28: 687-94), apoptotic protein Apaf-1 (Coldwell, M. J. et al. (2000) Oncogene 19: 899-905), DAP-5 (Henis-Korenblit, S. et al. (2000) Mol. Cell Bio. 20: 496-506), connexin (Werner, R. (2000) IUBMB Life 50: 173-76), Notch-2 (Lauring, S. A. et al. (2000) Mol. Cell. 6: 939-45), and fibroblast growth factor (Creancier, L. et al. (2000) J. Cell. Biol. 150: 275-81). As some IRES sequences act or function efficiently in particular cell types, the person skilled in the art will choose IRES elements with relevance to the particular cells being used to express the fusion nucleic acid. Moreover, multiple IRES sequences in various combinations, either homomultimeric or heteromultimeric arrangements constructed as tandem repeats or connected via linkers, are useful for increasing efficiency of translation initiation of the genes of interest. In a preferred embodiment, combinations of IRES elements comprise at least 2 to 10 or more copies or combinations of IRES sequences, depending on the efficiency of initiation desired.

In addition to their use as separation sequences, IRES elements serve as targets for therapeutic agents since IRES sequences mediate expression of proteins involved in viral pathogenesis (for example hepatitis C virus IRES sequences) or cellular disease states. Thus, the present invention is applicable in screens for candidate agents, such as random peptides, that inhibit IRES mediated translation initiation events.

Another preferred embodiment of IRES elements are sequences in nucleic acid or random nucleic acid libraries that function as IRES elements. Screens for these IRES type sequences can employ fusion nucleic acids containing bicistronically arranged genes of interest encoding reporter genes or selection genes, or combinations thereof. Genomic, cDNA, or random nucleic acid sequences are inserted between the two reporter or selection genes. After introducing the nucleic acid construct into cells, for example by retroviral delivery, the cells are screened for expression of the downstream gene mediated by a functional IRES sequence. Selection is based on expression of a downstream selection or reporter gene, for example, FACS analysis for expression of a downstream GFP gene. The upstream gene of interest serves to permit monitoring of expression of the fusion nucleic acid.

The length of the nucleic acids screened is preferably 6 to 100 nucleotides, although longer nucleic acids may be used.

The present invention further contemplates use of enhancers of IRES mediated translation initiation. IRES initiated translation may be enhanced by any number of methods. Cellular expression of virally encoded proteases that cleaves eIF4F to remove CAP-binding activity from the 40S ribosome complexes may be employed to increase preference for IRES translation initiation events. These proteases are found in some Picornaviruses and can be expressed in a cell by introducing the viral protease gene by transfection or retroviral delivery (Roberts, L. O. (1998) RNA 4: 520-29). Other enhancers adaptable for use with IRES elements include cis-acting elements, such as 3′ untranslated region of hepatitis C virus (Ito, T. et al. (1998) J. Virol. 72: 8789-96) and polyA segments (Bergamini, G. et al. (2000) RNA 6: 1781-90), which may be included as part of the fusion nucleic acid of the present invention. In addition, preferential use of cellular IRES sequences may occur when CAP dependent mechanisms are impaired, for example by dephosphorylation of 4E-BP, proteolytic cleavage of elF4G, or when cells are placed under stress by γ-irradiation, amino acid starvation, or hypoxia. Thus, in addition to the methods described above, IRES enhancing procedures include activation or introduction of 4E-BP targeted phosphatases or proteases of eIF4G. Alternatively, the cells are subjected to stress conditions described above. Other trans-acting IRES enhancers include heterogeneous nuclear ribonucleoprotein (hnRNP, Kaminski, A. et al. (1998) RNA 4: 626-38), PTB hnRNP E2/PCBP2 (Walter, B. L. et al. (1999) RNA 5: 1570-85), La autoantigen (Meerovitch, K. et al. (1993) J. Virol. 67: 3798-07), unr (Hunt, S. L. et al. (1999) Genes Dev. 13: 437-48), ITAF45/Mpp1 (Pilipenko, E. V. et al. (2000) Genes Dev. 14: 2028-45), DAP5/NAT1/p97 (Henis-Korenblit, S. et al. (2000) Mol. Cell. Biol. 20: 496-506), and nucleolin (Izumi, R. E. et al. (2001) Virus Res. 76: 17-29).

These factors may be introduced into a cell either alone or in combination. Accordingly, various combinations of IRES elements and enhancing factors are used to effect a separation reaction. In another preferred embodiment, the separation sites are Type 2A separation sequences. By “Type 2A” sequences herein is meant nucleic acid sequences that when translated inhibit formation of peptide linkages during the translation process. Type 2A sequences are distinguished from IRES sequences in that 2A sequences do not involve CAP independent translation initiation. Without being bound by theory, Type 2A sequences appear to act by disrupting peptide bond formation between the nascent polypeptide chain and the incoming activated tRNA^{PRO} (Donnelly, M. L. et al. (2001) J. Gen. Virol 82: 1013-25). Although the peptide bond fails to form, the ribosome continues to translate the remainder of the RNA to produce separate peptides unlinked at the carboxy terminus of the 2A peptide region. An advantage of Type 2A separation sequences is that near stoichiometric amounts of first protein of interest and second protein of interest are made as compared to IRES elements. Moreover, Type 2A sequences do not appear to require additional factors, such as proteases that are required to effect separation when using protease recognition sites. Although the exact mechanism by which Type 2A sequences function is unclear, practice of the present invention is not limited by the theorized mechanisms of 2A separation sequences. Preferred Type 2A separation sequences are those found in cardioviral and apthoviral genomes, which are approximately 21 amino acids long and have the general sequence XXXXXXXXXXLXXXDXEXNPGP, where X is any amino acid. Disruption of peptide bond formation occurs between the underlined carboxy terminal glycine (G) and proline (P). These 2A sequences are found, among others, in the apthovirus Foot and Mouth Disease Virus (FMDV), cardiovirus Theiler's murine encephalomyelitis virus (TME), and encephalomyocarditis virus (EMC). Various viral Type 2A sequences are known in the art. The 2A sequences function in a wide range of eukaryotic expression systems, thus allowing their use in a variety of cells and organisms. Accordingly, inserting these 2A separation sequences in between the nucleic acids encoding the first gene of interest and second gene of interest, as more fully explained below, will lead to expression of separate protein products of the first gene of interest and the second gene of interest.

In another embodiment, the present invention contemplates mutated versions or variants of Type 2A sequences. By “mutated” or “variant” or grammatical equivalents herein is meant deletions, insertions, transitions, transversions of nucleic acid sequences that exhibit the same qualitative separating activity as displayed by the naturally occurring analogue, although preferred mutants or variants have higher efficient separating activity and efficient translation of the downstream gene of interest. Mutant variants include changes in nucleic acid sequence that do not change the corresponding 2A amino acid sequence, but incorporate frequently used codons (i.e., codon optimized) to allow efficient translation of the 2A region (see Zolotukin, S. et al. (1996) J. Virol. 70: 4646-54). In another aspect, the mutant variants are changes in nucleic acid sequence that change the corresponding 2A amino acid sequence. In one aspect, preferred embodiments of variant 2A sequences are short deletions of the 20 amino acid 2A sequence that retains separating activity. The deletion may comprise removal of about 3 to 6 amino acids at the amino terminus of the 2A region. In another embodiment, Type 2A sequences are mutated by methods well known in the art, such as chemical mutagenensis, oligonucleotide directed mutagenesis, and error prone replication. Mutants with altered separating activity are readily identified by examining expression of the fusion nucleic acids of the present invention. Assaying for production of a separate downstream gene product, such as a reporter protein or a selection protein, allows for identifying sequences having separating activity. Another method for identifying variants may use a FRET based assay using linked GFP molecules, as described above. Insertion of variant 2A sequences in replace of or adjacent to the gly-ser linker region, or other suitable regions linking the GFPs will allow detection of functional 2A separation sequences by identifying constructs that produce separated GFP molecules, as measured by loss of FRET signal. Sequences having no or reduced separating activity will retain higher levels of FRET signal due to physical linkage of the GFP molecules. This strategy will permit high throughput analysis of variants and allows selecting of sequences having high efficiency Type 2A separating activity.

In yet another embodiment, Type 2A separation sequences include homologs present in other nucleic acids, including nucleic acids of other viruses, bacteria, yeast, and multicellular organisms such as worms, insects, birds, and mammals. Homology in this context means sequence similarity or identity. A variety of sequence based alignment methodologies, which are well known to those skilled in the art, are useful in identifying homologous sequences. These include, but not limited to, the local homology algorithm of Smith, F. and Waterman, M. S. (1981) Adv. Appl. Math. 2: 482-89, homology alignment algorithm of Peason, W. R. and Lipman, D. J. (1988) Proc. Natl. Acad. Sci. USA 85: 2444-48, Basic Local Alignment Search Tool (BLAST) described by Altschul, S. F. et al. (1990) J. Mol. Biol. 215: 403-10, or the Best Fit program described by Devereau, J. et al. (1984) Nucleic Acids. Res. 12: 387-95, and the FastA and TFASTA alignment programs, preferably using default settings or by inspection.

In one preferred embodiment, similarity or identity for any nucleic acid or protein outlined herein is calculated by Fast alignment algorithms based upon the following parameters: mismatch penalty of 1.0; gap size penalty of 0.33, joining penalty of 30 (see “Current Methods in Comparison and Analysis” in Macromolecule Sequencing and Synthesis: Seleted Methods and Applications, p. 12749, Alan R. Liss, Inc., 1998). Another example of a useful algorithm is PILEUP. PILEUP creates multiple sequence alignment from a group of related sequences using progressive, pairwise alignments. It can also plot a tree showing the clustering relationships used to create the alignment. PILEUP uses a simplification of the progressive alignment method of Feng, D. F. and Doolittle, R. F. (1987) J. Mol. Evol. 25, 351-60, which is similar to the method described by Higgins, D. G. and Sharp, P. M. (1989) CABIOS 5: 151-53. Useful parameters include a default gap weight of 3.00, a default gap length weight of 0.10, and weighted end gaps.

Another example of a useful algorithm is the family of BLAST alignment tools initial described by Altschul et al. (see also Karlin, S. et al. (1993) Proc. Natl. Acad. Sci. USA 90: 5873-87). A particularly useful BLAST program is WU-BLAST-2 program described in Altschul, S. F. et al. (1996) Methods Enzymol. 266: 460-80. WU-BLAST uses several search parameters, most of which are set to default values. The adjustable parameters are set with the following values: overlap span=1, overlap fraction=0.125, word threshold (T)=11. The HSP S and HSP S2 parameters are dynamic values and are established by the program itself depending upon composition of the particular sequence and composition of the particular database against which the sequence of interest is being searched; however, the values may be adjusted to increase sensitivity. A % amino acid sequence identity value is determined by the number of matching identical residues divided by the total number of residues of the longer sequence in the aligned region. The “longer” sequence is one having the most actual residues in the aligned region (gaps introduced by WU-BLAST-2 to maximize the alignment score are ignored).

In a similar manner, “percent (%) nucleic acid sequence identity” with respect to the coding sequence of the polypeptide described herein is defined as the percentage of the nucleotide residues in a candidate sequence that are identical with the nucleotide residues in the coding sequence of the Type 2A regions. A preferred method utilizes the BLASTN module of WU-BLAST-2 set to the default parameters, with overlap span and overlap fraction set to 1 and 0.125, respectively.

An additional useful algorithm is gapped BLAST as reported by Altschul, S. F. et al. (1997) Nucleic Acids Res. 25: 3389-402. Gapped BLAST uses BLOSSOM-62 substitution scores; threshold parameter set to 9; the two-hit method to trigger ungapped extensions; charges gap lengths of k at cost of 10+k; Xu set to 16, and Xg set to 40 for database search stage and to 67 for the output stage of the algorithms. Gapped alignments are triggered by a score corresponding to −22 bits.

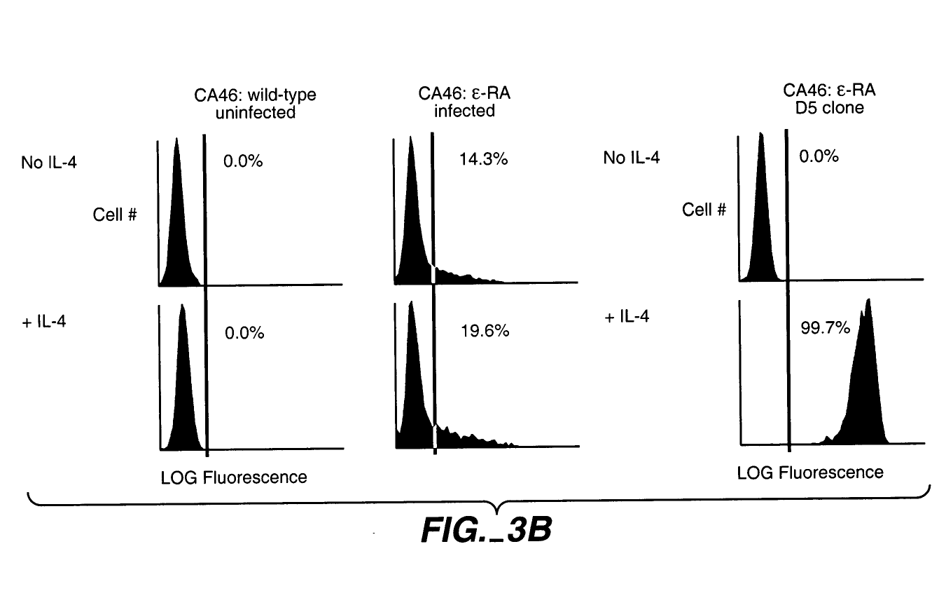

The alignment may include the introduction of gaps in the sequence to be aligned. In addition, for sequence which contain either more or fewer amino acids that the Type 2A sequences in FIG. 3, it is understood that the percentage of the homology will be determined based on the number of homologous amino acids in relation to the total number of amino acids. Thus, Type 2A sequences may be shorter or longer than the amino acid sequence shown in FIG. 3.

Another embodiment of Type 2A separating sequences are those sequences present in libraries of nucleic acids, including genomic DNA or cDNA that have Type 2A separating activity. By Type 2A separating activity herein is meant a nucleic acid which encodes a amino acid sequence that exhibits similar separating activity as the naturally occurring Type 2A sequences. Segments of nucleic acids are inserted between the first gene of interest and second gene of interest in the fusion nucleic acids of the present invention and examined for separating activity as described above. The preferred lengths to be tested are nucleic acids encoding peptides of about 5 to 50 amino acids or larger, with a more preferred range of peptides of about 10-30 amino acids long.

Embodiments of Type 2A sequence also encompass random nucleic acids encoding random peptides that have Type 2A separating activity. In these embodiments, the separation site represents a randomizing region where random or biased random nucleic acids encoding random or biased random peptides are inserted between the first gene of interest and second gene of interest. The preferred lengths of the random nucleic acids are nucleic acids encoding peptides 5 to 50 amino acids, with a more preferred range of peptides 10-30 amino acids. Random peptides having separating activity are identified using the above described assays. Identification of functional separating sequences will permit additional searches for related sequences having Type 2A like separating activity, either through homology searches, mutagenesis screens, or by use of biased random peptide sequences. Sequences with separating activity can then be used to express separate proteins of interest according to the present invention.

In a preferred embodiment, the fusion nucleic acids of the present invention further comprises genes of interest linked to a fusion partner to form a fusion polypeptide. By fusion partner or functional group herein is meant a sequence that is associated with the gene of interest, or candidate agent described below, that confers upon all members of the library in that class a common function or ability. Fusion partners can be heterologous (i.e., not native to the host cell), or synthetic (i.e., not native to any cell). Suitable fusion partners include, but are not limited to: (a) presentation structures, as defined below, which provide the peptides of interest and candidate agents in a conformationally restricted or stable form; (b) targeting sequences, defined below, which allow the localization of the genes of interest and candidate agent into a subcellular or extracellular compartment; (c) rescue sequences as defined below, which allow the purification or isolation of either the peptide of interest (for example, when a gene of interest encodes a peptide) or candidate agents or the nucleic acids encoding them; (d) stability sequences, which affects the stability or degradation to the protein of interest or candidate agent or the nucleic acid encoding it, for example resistance or susceptibility to proteolytic degradation; (e) dimerization sequences, to allow for peptide dimerization; or (f) any combination of the above, as well as linker sequences as needed.

In a preferred embodiment, the fusion partner is a presentation structure. By “presentation structure” or grammatical equivalents herein is meant a sequence, when fused to a peptide encoded by gene of interest or peptide candidate agents, causes the peptides to assume a conformationally restricted form. Proteins interact with each other largely through conformationally constrained domains. Although small peptides with freely rotating amino and carboxyl termini can have potent functions as is known in the art, the conversion of such peptide structures into pharmacologic or biologically active agents is difficult due to the inability to predict side-chain positions for peptidomimetic synthesis. Therefore the presentation of peptides in conformationally constrained structures will benefit both the later generation of pharmaceuticals and will also likely lead to higher affinity interactions of the peptide with the target protein. This fact has been recognized in the combinatorial library generation systems using biologically generated short peptides in bacterial phage systems. A number of workers have constructed small domain molecules in which one might present short peptide domains or randomized peptide structures.

Presentation structures are preferably used with peptides encoded by genes of interest and peptide candidate agents encoded by random nucleic acids, although candidate agents, as more fully described below, may be either nucleic acid or peptides. Thus, when presentation structures are used with peptide candidate agents, synthetic presentation structures, i.e., artificial polypeptide, are adaptable for presenting a peptide, for example a randomized peptide, as a conformationally-restricted domain. Generally, such presentation structures comprise a first portion joined to the N-terminal end of the peptide, and a second portion joined to the C-terminal end of the peptide; that is, the peptide is inserted into the presentation structure, although variations may be made, as outlined below. To increase the functional isolation of the peptide expression product, the presentation structures are selected or designed to have minimal biologically activity when expressed in the target cell.

Preferred presentation structures maximize accessibility to the peptide by presenting it on an exterior loop. Accordingly, suitable presentation structures include, but are not limited to, minibody structures, loops on beta-sheet turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc.

In a preferred embodiment, the presentation structure is a coiled-coil structure, allowing the presentation of the protein or randomized peptide on an exterior loop (Myszka, D. G. et al. (1994) Biochemistry 33: 2362-73, hereby incorporated by reference). Using this system investigators have isolated peptides capable of high affinity interaction with the appropriate target. In general, coiled-coil structures allow for between 6 to 20 randomized positions.

A preferred coiled-coil presentation structure is as follows:

MGCAALESEVSALESEVASLESEVAALGRGDMPLAAVKSKLSAVKSKLASVKSKLAACGPP. The underlined regions represent a coiled-coil leucine zipper region defined previously (Martin, F. et al. (1994) EMBO J. 13: 5303-09, hereby incorporated by reference). The bolded GRGDMP region represents the loop structure and may be appropriately replaced with gene of interest (e.g., randomized peptides or peptide interaction domains), generally depicted herein as (X)ₙ, where X is an amino acid residue and n is an integer of at least 5 or 6 and of variable length. The replacement of the bolded region is facilitated by encoding restriction endonuclease sites in the underlined regions, which allows the direct incorporation of genes of interest or randomized oligonucleotides at these positions. For example, a preferred embodiment generates a XhoI site at the double underlined LE site and a HindIII site at the double-underlined KL site.

In a preferred embodiment, the presentation structure is a minibody structure. A “minibody” is essentially composed of a minimal antibody complementarity region. The minibody presentation structure generally provides two sites for insertion of peptides or for randomizing amino acids that in the folded protein are presented along a single face of the tertiary structure (see for example, Bianchi, E. et al. (1994) J. Mol. Biol. 236: 649-59, and references cited therein, all of which are incorporated by reference). Investigators have shown this minimal domain is stable in solution and have used phage selection systems in combinatorial libraries to select minibodies with peptide regions exhibiting high affinity (K_{d}=10⁻⁷) for the pro-inflammatory cytokine IL-6.

A preferred minibody presentation structure is as follows: MGRNSQATSGFTFSHFYMEWVRGG EYIAASRHKHNKYTTEYSASVKGRYIVSRDTSQSI LYLQKKKG PP. The bold, underlined regions are the regions which may be randomized. The italized phenylalanine must be invariant in the first randomizing region. The entire peptide is cloned in a three-oligonucleotide variation of the coiled-coil embodiment, thus allowing two different randomizing regions to be incorporated simultaneously. This embodiment utilizes non-palindromic BstXI sites on the termini.

In a preferred embodiment, the presentation structure is a sequence that contains generally two cysteine residues, such that a disulfide bond may be formed, resulting in a conformationally constrained sequence. This embodiment is particularly preferred when secretory targeting sequences are used. As will be appreciated by those in the art, any number of random peptide sequences, with or without spacer or linking sequences, may be flanked with cysteine residues. In other embodiments, effective presentation structures may be generated by the random regions themselves. For example, the random regions may be “doped” with cysteine residues which, under the appropriate redox conditions, may result in highly cross-linked structured conformations, similar to a presentation structure. Similarly, the randomization regions may be controlled to contain a certain number of residues to confer β-sheet or a-helical structures.

In a preferred embodiment, the presentation sequence confers the ability to bind metal ions to confer secondary structure. For example, C2H2 zinc finger sequences may be used; C2H2 sequences have two cysteines and two histidines placed such that a zinc ion is chelated. Zinc finger domains are known to occur independently in multiple zinc-finger peptides to form structurally independent, flexibly linked domains (see Nakaseko, Y. et al. (1992) J. Mol. Biol. 228: 619-36). A general consensus sequence is (5 amino acids)-C-(2 to 3 amino acids)-C-(4 to 12 amino acids)-H-(3 amino acids)-H-(5 amino acids). A preferred example would be -FQCEEC-peptide of 3 to 20 amino acids-HIRSHTG-.

Similarly, CCHC boxes can be used, that have a consensus seqeunce -C-(2 amino acids)-C-(4 to 20 peptide or random peptide)-H-(4 amino acids)-C- (see Bavoso, A. et al. (1998) Biochem. Biophys. Res. Commun. 242: 385-89, hereby incorporated by reference). Preferred examples include: (1)-VKCFNC-4 to 20 amino acid peptide-HTARNCR-, based on the nucleocapsid protein P2; (2) a sequence modified from that of the naturally occurring zinc-binding peptide of the Lasp-1 LIM domain (Hammarstrom, A. et al. (1996) Biochemistry 35:12723-32); and (3)-MNPNCARCG-4 to 20 amino acid peptide-HKACF-, based on the NMR structural ensemble 1ZFP (Hammarstrom et al., supra).

In a preferred embodiment, the fusion partner is a targeting sequence. As will be appreciated by those in the art, the localization of proteins within a cell is a simple method for increasing effective concentration and determining function. For example, RAF-1 targeted to the mitochondrial membrane can inhibit the anti-apoptotic effect of BCL-2. Similarly, membrane bound Sos induces Ras mediated signaling in T-lymphocytes. These mechanisms are thought to rely on the principle of limiting the search space for ligands; that is to say, the localization of a protein to the plasma membrane limits the search for its ligand to that limited dimensional space near the membrane as opposed to the three dimensional space of the cytoplasm. Alternatively, the concentration of a protein can also be simply increased by nature of the localization. Shuttling the proteins into the nucleus confines them to a smaller volume thereby increasing concentration. Finally, the ligand or target may simply be localized to a specific compartment, and cognate inhibitors localized appropriately.

Thus, suitable targeting sequences include, but are not limited to, affinity sequences capable of causing binding of the expression product to a predetermined molecule or class of molecules while retaining bioactivity of the expression product, (for example by using enzyme inhibitor or substrate sequences to target a class of relevant enzymes); sequences signaling selective degradation, of itself or co-bound proteins; and signal sequences capable of constitutively localizing the candidate expression products to a predetermined cellular locale, including (a) subcellular locations such as the Golgi, endoplasmic reticulum, nucleus, nucleoli, nuclear membrane, mitochondria, chloroplast, secretory vesicles, lysosome, and cellular membrane; and (b) extracellular locations via a secretory signal. Particularly preferred is localization to either subcellular locations or to the outside of the cell via secretion.

In a preferred embodiment, the targeting sequence is a nuclear localization signal (NLS). NLSs are generally short, positively charged (basic) domains that serve to direct the entire protein in which they occur to the cell's nucleus. Numerous NLS amino acid sequences have been reported including single basic NLS's such as that of SV40 (monkey virus) large T Antigen (PKKKRKV, Kalderon, D. et al. (1984) Cell 39: 499-509); the human retinoic acid receptor-β nuclear localization signal (ARRRRP), NFKB p50 (EEVQRKRQKL, Ghosh, S. et al. (1990) Cell 62:1019-29); NFKB p65 (EEKRKRTYE, Nolan, G. et al. (1991) Cell 64: 961-99; and others (see for example Boulikas, T. (1994) J. Cell. Biochem. 55: 32-58, hereby incorporated by reference) and double basic NLS's exemplified by that of the Xenopus (African clawed toad) protein, nucleoplasmin (AVKRPAATKKAGQAKKKKLD, Dingwall, C. et al. (1982) Cell, 30: 449-58, and Dingwall, S. et al. (1988) J. Cell Biol. 107: 641-49). Numerous localization studies have demonstrated that NLSs incorporated in synthetic peptides or grafted onto proteins not normally targeted to the cell nucleus cause these peptides and proteins to concentrate in the nucleus (see Dingwall S. et al. (1986) Ann. Rev. Cell Biol. 2: 367-90; Bonnerot, C. et al. (1987) Proc. Natl. Acad. Sci. USA 84: 6795-99; and Galileo, D. S. et al. (1990) Proc. Natl. Acad. Sci. USA 87: 458-62.)

In a preferred embodiment, the targeting sequence is a membrane anchoring signal sequence. These sequences are particularly useful since many intracellular events originate at the plasma membrane and many parasites and pathogens bind to the membrane during pathogenesis. Thus, membrane-bound peptide libraries are useful for both for the identification of important elements in these processes as well as for the discovery of effective inhibitors. The invention provides methods for presenting the peptide encoded by gene of interest or randomized peptide candidate agent extracellularly or in the cytoplasmic space. For extracellular presentation, a membrane anchoring region is provided at the carboxyl terminus of the peptide presentation structure. The peptide or randomized expression product region is expressed on the cell surface and presented to the extracellular space, such that it can bind to other surface molecules (affecting their function) or molecules present in the extracellular medium. The binding of such molecules could confer function on the cells expressing a peptide that binds the molecule. The cytoplasmic region could be neutral or could contain a domain that, when the extracellular expression product region is bound, confers a function on the cells (activation of a kinase, phosphatase, binding of other cellular components to effect function). Similarly, a region containing the peptide of interest or randomized peptide could be confined within the cytoplasmic compartment and the transmembrane region and extracellular region remain constant or have specified function.

Membrane-anchoring sequences are well known in the art and are based on the genetic geometry of mammalian transmembrane molecules. Peptides are inserted into the membrane via a signal sequence (designated herein as ssTM) and stably held in the membrane through a hydrophobic transmembrane domain (TM). The transmembrane proteins are positioned in the membrane such that the protein region encompassing the amino terminus relative to the transmembrane domain are extracellular and the region towards the carboxy terminal are intracellular. Of course, if the position of transmembrane domains is towards the amino end of the protein relative to the peptide of interest, the TM will serve to position the peptide of interest intracellularly, which may be desirable in some embodiments. ssTMs and TMs are known for a wide variety of membrane bound proteins, and these sequences are used accordingly, either as pairs from a particular protein or with each component being taken from a different protein. Alternatively, the ssTM and TM sequences are synthetic and derived entirely from consensus sequences, thus serving as artificial delivery domains.

As will be appreciated by those in the art, membrane-anchoring sequences, including ssTM and TM, are known for a wide variety of proteins and any of these are useful in the present invention. Particularly preferred membrane-anchoring sequences include, but are not limited to, those derived from CD8, ICAM-2, IL-8R, CD4 and LFA-1. Other useful ssTM and TM domains include sequences from: (a) class I integral membrane proteins such as IL-2 receptor beta-chain (residues 1-26 are the signal sequence, 241-265 are the transmembrane residues; see Hatakeyama, M. et al. (1989) Science 244: 551-56 and von Heijne, G. et al. (1988) Eur. J. Biochem. 174: 671-78) and insulin receptor beta chain (residues 1-27 are the signal domain, 957-959 are the transmembrane domain and 960-1382 are the cytoplasmic domain; see Hatakeyama et al., supra, and Ebina, Y. et al. (1985) Cell 40: 747-58); (b) class 11 integral membrane proteins such as neutral endopeptidase (residues 29-51 are the transmembrane domain, 2-28 are the cytoplasmic domain; see Malfroy, B. et al. (1987) Biochem. Biophys. Res. Commun. 144: 59-66); (c) type III proteins such as human cytochrome P450 NF25 (Hatakeyama et al., supra); and (d) type IV proteins such as human P-glycoprotein (Hatakeyama et al., supra). Particularly preferred are CD8 and ICAM-2. For example, the signal NF5 sequences from CD8 and ICAM-2 lie at the extreme 5′ end of the transcript. These consist of the amino acids 1-32 in the case of CD8 (MASPLTRFLSLNLLLLGESILGSGEAKPQAP, Nakauchi, H. et al. (1985) Proc. Natl. Acad. Sci. USA 82: 5126-30) and amino acid 1-21 in the case of ICAM-2 (MSSFGYRTLTVALFTLICCPG, Staunton, D. E. et al. (1989) Nature 339: 61-64). These leader sequences deliver the construct to the membrane while the hydrophobic transmembrane domains placed at the carboxy terminal region relative to the peptide of interest or peptide candidate agents serve to anchor the construct in the membrane. These transmembrane domains are encompassed by amino acids 145-195 from CD8 (PQRPEDCRPRGSVKGTGLDFACDIYIWAPLAGICVALLLSLIITLICYHSR, Nakauchi et al., supra) and 224-256 from ICAM-2 (MVIIVTVVSVLLSLFVTSVLLCFIFGQHLRQQR, Staunton et al., supra).

Alternatively, membrane anchoring sequences include the GPI anchor, which results in a covalent bond between the molecule and the lipid bilayer via a glycosyl-phosphatidylinositol bond. The GPI anchor sequence is exemplified by protein DAF, which comprises the sequence PNKGSGTTSGTTRLLSGHTCFTLTGLLGTLVTMGLLT, with the bolded serine the site of the anchor; (see Homans, S. W. et al. (1988) Nature 333: 269-72, and Moran, P. et al. (1991) J. Biol. Chem. 266: 1250-57). Adding GPI anchor sites is accomplished by inserting the GPI sequence from Thy-1 in the carboxy terminal region relative the inserted peptide of interest or randomized peptide. Thus, the GPI anchor sequences replaces the transmembrane domain in these constructs.

Similarly, acylation signals for attachment of lipid moieties can also serve as membrane anchoring sequences (see Stickney, J. T. (2001) Methods Enzymol. 332: 64-77). It is known that the myristylation of c-src localizes the kinase to the plasma membrane. This property provides a simple and effective method of membrane localization given that the first 14 amino acids of the protein are solely responsible for this function: MGSSKSKPKDPSQR (see Cross, F. R. et al. (1984) Mol. Cell. Biol. 4: 1834-42; Spencer, D. M. et al. (1993) Science 262: 1019-24, both of which are hereby incorporated by reference) or MGQSLTTPLSL. The modification at the glycine residue (in bold) of the motif is effective in localizing reporter genes and can be used to anchor the zeta chain of the TCR. The myristylation signal motif is placed at the amino end relative to the peptide or protein of interest in order to localize the construct to the plasma membrane. Another lipid modification is isoprenoid attachment, which includes the 15 carbon farnesyl or the 20 carbon geranyl-geranly group. The conserved sequence for isoprenoid attachment comprises CaaX motif with the cysteine residue as the lipid modified amino acid. The X residue determines the type of isoprenoid modification. The preferred isoprenoid is geranyl-geranyl when X is a leucine or phenylalanine (Farnsworth, C. C. et al. (1994) Proc. Natl. Acad. Sci. USA 91: 11963-67). Farnesyl is the preferred lipid for a broader range of X amino acids such as methionine, serine, glutamine and alanine. The “aa” in the isoprenoid attachment motif are generally aliphatic residues, although other residues are also functional. Farnesylation sequences include carboxy terminal SKDGKKKKKKSKTKCVIM of K-Ras4B. Other isoprenoid attachment motifs are found in the carboxy termini of N and H-Ras GTPases.

In addition, localization to the cell membrane by lipid modification is also achieved by palmitoylation. Attachment of the palmitoyl group can be directed to either the amino or carboxy terminal region relative to the protein of interest. In addition, multiple palmitoyl residues or combinations of palmitoyl and isoprenoids are possible. Amino terminal additions of palmitoyl group may use the sequence MVCCMRRTKQV from Gap43 protein while carboxy terminal modifications are possible with CMSCKCVLKKKKKK from Ras mutant (modified amino acids in bold). Other palmitoylation sequences are found in G protein-coupled receptor kinase GRK6 sequence (LLQRLFSRQDCCGNCSDSEEELPTRL, Stoffel, R. H. et al. (1994) J. Biol. Chem. 269: 27791-94); rhodopsin (KQFRNCMLTSLCCGKNPLGD, Barnstable, C. J. et al. (1994) J. Mol. Neurosci. 5: 207-09); and the p21H-ras 1 protein (LNPPDESGPGCMSCKCVLS, Capon, D. J. et al. (1983) Nature 302: 33-37). Use of the carboxy terminal sequence LNPPDESGPGC(p)MSC(p)KC(f)VLS of H-Ras (modified amino acids in bold; p is palmitoyl group and f is farnesyl group) allows attachment of both palmitoyl and farnesyl lipids

In a preferred embodiment, the targeting sequence is a lysozomal targeting sequence, including, for example, a lysosomal degradation sequence such as Lamp-2 (KFERQ, Dice, J.F. (1992) Ann. N.Y. Acad. Sci. 674: 58-64); or lysosomal membrane sequences from Lamp-1 (MLIPIAGFFALAGLVLIVLIAYLIGRKRSHAGYQTI, Uthayakumar, S. et al. (1995) Cell. Mol. Biol. Res. 41: 405-20) or Lamp-2 (LVPIAVGAALAGVLILVLLAYFIGLKHHHAGYEQF, Konecki, D. S. et al. (1994) Biochem. Biophys. Res. Comm. 205: 1-5; where italicized residues comprise the transmembrane domains and underlined residues comprise the cytoplasmic targeting signal).

Alternatively, the targeting sequence may be a mitochondrial localization sequence, including mitochondrial matrix sequences (e.g. yeast alcohol dehydrogenase III; MLRTSSLFTRRVQPSLFSRNILRLQST, Schatz, G. (1987) Eur. J. Biochem. 165:1-6); mitochondrial inner membrane sequences (yeast cytochrome c oxidase subunit IV; MLSLRQSIRFFKPATRTLCSSRYLL, Schatz, supra); mitochondrial intermembrane space sequences (yeast cytochrome c1; MFSMLSKRWAQRTLSKSFYSTATGAASKSGKLTQKLVTAGVAAAGITASTLLYADSLTAEAMTA, Schatz, supra) or mitochondrial outer membrane sequences (yeast 70 kD outer membrane protein; MKSFITRNKTAILATVMTGTAIGAYYYYNQLQQQQQRGKK, Schatz, supra).

The target sequences may also be endoplasmic reticulum sequences, including the sequences from calreticulin (KDEL, Pelham, H.R. (1992) Royal Society London Transactions B; 1-10) or adenovirus E3/19K protein (LYLSRRSFIDEKKMP, Jackson, M. R. et al. (1990) EMBO J. 9: 3153-62). Furthermore, targeting sequences also include peroxisome sequences (for example, the peroxisome matrix sequence of luciferase, SKL (Keller, G. A. et al. (1987) Proc. Natl. Acad. Sci. USA 4: 3264-68); or destruction sequences (e.g., cyclin B1, RTALGDIGN; Klotzbucher, A. et al. (1996) EMBO J. 1: 3053-64).

In a preferred embodiment, the targeting sequence is a secretory signal sequence capable of effecting the secretion of the peptide of interest or peptide candidate agent. There are a large number of known secretory signal sequences which direct secretion of the peptide into the extracellular space when placed at the amino end relative to the peptide of interest. Secretory signal sequences and their transferability to unrelated proteins are well known (see Silhavy, T. J. et al. (1985) Microbiol. Rev. 49: 398-418). Secretion of the peptide is particularly useful to generate peptides capable of binding to the surface of, or affecting the physiology of, a target cells other than the host cell, e.g., the cell infected with the retrovirus. In a preferred approach, a fusion product is configured to contain, in series, secretion signal peptide-presentation structure-randomized peptide region or protein of interest-presentation structure. In this manner, target cells grown in the vicinity of cells expressing the library of peptides are exposed to the secreted peptide. Target cells exhibiting a physiological change in response to the presence of the secreted peptide (i.e., by the peptide binding to a surface receptor or by being internalized and binding to intracellular targets) and the peptide secreting cells are localized by any of a variety of selection schemes and the structure of the peptide effector identified. Exemplary effects include that of a designer cytokine (e.g., a stem cell factor capable of causing hematopoietic stem cells to divide and maintain their totipotential), a factor causing cancer cells to undergo spontaneous apoptosis, a factor that binds to the cell surface of target cells and labels them specifically, etc.

Suitable secretory sequences are known, including signals from IL-2 (MYRMQLLSCIALSLALVTNS; Villinger, F. et al. (1995) J. Immunol. 155: 3946-54), growth hormone (MATGSRTSLLLAFGLLCLPWLQEGSAFPT; Roskam, W. G. et al. (1979) Nucleic Acids Res. 7: 305-20); preproinsulin (MALWMRLLPLLALLALWGPDPAAAFVN; Bell, G. I. et al. (1980) Nature 284: 26-32); and influenza HA protein (MKAKLLVLLYAFVAGDQI, Sekiwawa, K. et al. (1983) Proc. Natl. Acad. Sci. USA 80: 3563-67), with cleavage between the non-underlined-underlined junction. A particularly preferred secretory signal sequence is the signal leader sequence from the secreted cytokine IL-4, MGLTSQLLPPLFFLLACAGNFVHG, which comprises the first 24 amino acids of IL-4.

In a preferred embodiment, the fusion partner is a rescue sequence. A rescue sequence is a sequence which may be used to purify or isolate either the peptide of interest or the candidate agent or the nucleic acid encoding it. Thus, for example, peptide rescue sequences include purification sequences such as the His₆ tag for use with Ni⁺² affinity columns and epitope tags useful for detection, immunoprecipitation or FACS (fluoroscence-activated cell sorting). Suitable epitope tags include myc (for use with the commercially available 9E10 antibody), the BSP biotinylation target sequence of the bacterial enzyme BirA, flu tags, lacZ, GST, and Strep tag I and II.

Alternatively, the rescue sequence may be a unique oligonucleotide sequence which serves as a probe target site to allow the facile isolation of the retroviral construct, via PCR, related techniques, or by hybridization.

In a preferred embodiment, the fusion partner is a stability sequence to affects the stability to the peptide of interest or candidate bioactive agent. In one aspect, the stability sequence confers stability to the peptide of interest or candidate bioactive agent. For example, peptides may be stabilized by the incorporation of glycines after the initiating methionine (MG or MGG), for protection of the peptide to ubiquitination as per Varshavsky's N-End Rule, thus conferring increased half-life in the cell (see Varshavsky, A. (1996) Proc. Natl. Acad. Sci. USA 93: 12142-49). Similarly, adding two prolines at the C-terminus makes peptides that are largely resistant to carboxypeptidase action. The presence of two glycines prior to the prolines impart both flexibility and prevent structure perturbing events in the di-proline from propagating into the peptide structure. Thus, preferred stability sequences are MG(X)ₙGGPP, where X is any amino acid and n is an integer of at least four.

In another aspect, the stability sequence decreases the stability of the peptide of interest or candidate bioactive agent. Sequences, such as PEST sequences (polypeptide sequences enriched in proline (P), glutamic acid (E), serine (S) and threonine (T); see Rechsteiner, M. (1996) Trends Biochem. Sci. 21: 267-71) and destruction boxes (Glotzer, M. (1991) Nature 349 132-38) destabilize proteins by targeting proteins for degradation. For example, fusion of PEST sequences to GFP reporter protein decreases the half-life of GFP, thus providing a indicator of dynamic cellular processes, including, but not limited to, regulated protein degradation, reporter for transcriptional activity, and cell cycle status (Mateus, C. et al. (2000) Yeast 16:1313-23; Li. X. (1998) J. Biol. Chem. 273: 34970-75). Numerous PEST sequences useful for targeting peptides for degradation are known. These include amino acids 422-461 of ornithine decarboxylase (Corish, P. (1999) Protein Eng. 12: 1035-40) and the C terminal sequences of IκBα (Lin, R. (1996) Mol. Cell Biol. 16: 1401-09). Destruction boxes found in cell cycle proteins, for example cyclin B1, can also reduce the half-life of fusion proteins but in a cell cycle dependent manner (Corish, supra).

In another embodiment, the fusion partner is a multimerization sequence. A multimerization sequence allows non-covalent association of one peptide of interest to another peptide of interest, with sufficient affinity to remain associated under normal physiological conditions. This effectively allows small libraries of peptides encoded by genes of interest or peptide candidate agents (for example, 10⁴) to become large libraries if, for example, two peptides per cell are generated which then dimerize, to form an effective library of 10⁸ (10⁴×10⁴). It also allows the formation of longer random peptides, if needed, or more structurally complex random peptide molecules. The multimers may be homo- or heteromeric. One preferred multimerization sequences are dimerization sequences.

Dimerization or multimerization sequences may be a single sequence that self-aggregates, or two sequences, each of which is present in the fusion nucleic acid comprising first gene of interest and second gene of interest. Alternatively, the multimerization sequences are present in different retroviral constructs, with each construct expressing a different gene of interest with multimerization sequences. Thus, in various embodiments, nucleic acids encode a first peptide with dimerization sequence 1, and a second peptide with dimerization sequence 2, such that upon introduction into a cell and expression of the nucleic acids, dimerization sequence 1 associates with dimerization sequence 2 to form a new peptide structure or peptide candidate agent. Alternatively, two or more different multimerization sequences may be incorporated into individual gene of interest or candidate peptide agent. For example, a first multimerization sequence may be placed at the amino terminus while a second multimerization sequence is placed at the carboxy terminus. Expression of the protein or peptide allows formation of a variety of complex multiprotein associations, including protein concatemers. Moreover, the use of dimerization sequences allows the noncovalent “constraint” of the random peptides; that is, if a dimerization sequence is used at each terminus of the peptide, the resulting structure can form a constrained structure. Furthermore, the use of dimerizing sequences fused to both the N- and C-terminus of the scaffold such as rGFP or pGFP forms a noncovalently constrained scaffold random peptide library.

Suitable dimerization sequences will encompass a wide variety of sequences. Any number of protein-protein interaction sites are known. In addition, dimerization sequences may also be elucidated using standard methods such as the yeast two hybrid system, traditional biochemical affinity binding studies, or methods described in WO 99/51625, hereby incorporated by reference in its entirety. Particularly preferred dimerization peptide sequences include, but are not limited to, -EFLIVKS-, EEFLIVKKS-, -FESIKLV-, and -VSIKFEL-. More preferred dimerization peptide sequences include EEEFLIVEEE when used together with KKKFLIVKKK.

The fusion partners may be placed anywhere (i.e., N-terminal, C-terminal, internal) in the structure as the biology and activity permits.

In a preferred embodiment, the fusion partner includes a linker or spacer sequence. Linker sequences between various targeting sequences (for example, membrane targeting sequences) and the other components of the constructs (such as the randomized peptides) may be desirable to allow the peptides to interact with potential targets unhindered. For example, useful linkers include glycine polymers (G)ₙ, glycine-serine polymers (including, for example, (GS)ₙ, (GSGGS)ₙ and (GGGS)ₙ, where n is an integer of at least one), glycine-alanine polymers, alanine-serine polymers, and other flexible linkers such as the tether for the Shaker potassium channel, and a large variety of other flexible linkers, as will be appreciated by those in the art. Glycine and glycine-serine polymers are preferred since both of these amino acids are relatively unstructured, and therefore may be able to serve as a neutral tether between components. Glycine polymers are the most preferred as glycine accesses significantly more phi-psi space than even alanine, and is much less restricted than residues with longer side chains (see Scheraga, H. A. (1992) Rev. Computational Chem. 111 73-142). Secondly, serine is hydrophilic and therefore able to solubilize what could be a globular glycine chain. Third, similar chains have been shown to be effective in joining subunits of recombinant proteins such as single chain antibodies.

In addition, the fusion partners, including presentation structures, may be modified, randomized, and/or mutated to alter the presented or displayed orientation of the randomized expression product. For example, determinants at the base of the loop may be modified to slightly modify the internal loop peptide tertiary structure in order to properly display a randomized amino acid sequence.

In a preferred embodiment, combinations of fusion partners are used. Thus, for example, any number of combinations of presentation structures, targeting sequences, rescue sequences, and stability sequences may be used, with or without linker sequences. By using a base vector that contains a cloning sites for receiving libraries of genes of interest or candidate agents, one can cassette in various fusion partners 5′ and 3′ of the library. As will be appreciated by those in the art, these modules of sequences can be used in a large number of combinations and variations. In addition, as discussed herein, it is possible to have more than one variable peptide region in a construct, either together to form a new surface or to bring two other molecules together. Alternatively, no presentation structure is used, giving a “free” or “non-constrained” peptide or expression product.

Accordingly, in one preferred embodiment of the present invention, the first gene of interest may be a nucleic acid which encodes a fusion protein comprising a first fusion partner and a first reporter gene and the second gene of interest comprises a second fusion protein comprising a second fusion partner and second reporter gene. If the fusion partners comprise different cellular localization sequences, such as nuclear localization and membrane localization sequences, the presence of a separation sequence between the first and second gene of interest results in synthesis of separate proteins products capable of localizing to different cellular structures. For example, the described construct allows detecting cells by the nuclearly localized first fusion protein while permitting analysis of cellular morphology or cellular processes by the membrane localized second reporter gene. In complex cell cultures, such as hippocampal slices used for examining the basis for learning and memory and synaptic plasticity, tracing the neuronal projections of specific neuronal cells types is particularly important. The described construct allows identifying particular cells by the nuclearly localized first reporter gene and tracing of neuronal projections by the second reporter gene. Those skilled in the art will appreciate that use of different combinations of fusion partners and genes of interest permits monitoring of multiple cellular processes simultaneously. Similarly, targeting of proteins of interest to distinct cellular locations, either internal or external to the cell, is useful in directing proteins to regions where they will be biologically active.

As will be appreciated by those skilled in the art, any number of separating sequences and genes of interest may be used in the SIN vectors of the present invention. Additional separating sequences may be chosen from protease based, IRES based, or Type 2A based separating sequences and added to the fusion nucleic acids along with additional genes of interest. Accordingly, fusion nucleic acids of the present invention may further comprise a plurality of separating sequences and a plurality of genes of interest. The preferred embodiments include fusion nucleic acids further comprising a second separating sequence and a third gene of interest, and additionally a third separating sequence and a fourth additional gene of interest. As can be appreciated by those skilled in the art, by inserting additional separating sequences and additional genes of interest to the nucleic acids of the present invention, any number of proteins encoded by genes of interested may be separately expressed by the fusion nucleic acid. The additional genes of interest may be identical or non-identical to the first and second genes of interest. Additional separating sequences and gene of interest may be desired in screening methods where the first and second gene of interest encode reporter proteins whose activity is affected by an expressed third gene of interest or where expression of more than two genes of interest are necessary to produce a cellular effect.

The SIN vectors and the fusion nucleic acids of the present invention described herein can be prepared using standard recombinant DNA techniques described in, for example, Sambrook, J. et al., Molecular Cloning; A Laboratory Manual, 2nd edition, Cold Spring Harbor Press, Cold Spring Harbor, N.Y., 1989, and Ausubul, F. et al., Current Protocols in Molecular Biology, Greene Publishing Associates and John Wiley & Sons, New York, N.Y., 1994.

Preferred SIN vectors may be based on the murine stem cell virus (MSCV) (see Hawley, R. G. et al. (1994) Gene Ther. 1: 136-38), a modified MFG virus (Riviere, I. et al. (1995) Genetics 92: 6733-37), or pBABE. Other useful retroviral vectors for generating SIN vectors include, among others, LRCX retroviral vector set; pSIR retroviral vector; pLEGFP-NI retroviral vector, pLAPSN retroviral vector; pLXIN retroviral vector; and pLXSN retroviral vector; all of which are commercially available (i.e. Clontech). SIN vectors based on Moloney murine leukemia viruses have been described (Yu, S-F. et al. (1986) Proc. Natl. Acad. Sci. USA 83: 3194-98; Hoffman, A. (1996) Proc. Natl. Acad. Sci. USA 93: 5158-90; Hwang, J-J. et al. (1997) J. Virol. 71: 7128-31).

Since SIN vectors have inefficient or inactivated viral promoters needed for expressing the RNA for packaging into retroviral particles, the retroviral vectors generally contain additional promoter elements near the 5′ LTR to allow efficient expression of the RNAs packaged into viral particles. Situating these additional promoter sequences outside the 5′ U5 region results in absence of these elements in the packaged viruses, and their absence in the integrated proviral form of the retroviral vectors (see Naviaux, R. K. et al. (1996) J. Virol. 70: 5701-05).

When target cells are non-proliferating (e.g., brain cells), useful retroviral SIN vectors are derived from lentiviruses since these viruses, such as HIV virus, are capable of infecting both dividing and non-dividing cells. Self-inactivating retroviral vectors based on HIV viruses and related packaging methods are known in the art (see Miyoshi, H. (1998) J. Virol. 72: 8150-57; Zufferey, R. (1998) J. Virol. 72: 9873-80; Iwakuma, T. (1999) Virology 261: 120-32; Xu, K. (2001) Mol. Ther. 3: 97-104).

Generally, the SIN vectors also contain a number of other elements, including for example, the required regulatory sequences (e.g., translation, transcription, polyadenylation sites, etc), fusion partners, restriction endonuclease (cloning and subcloning) sites, stop codons preferably in all three frames, regions of complementarity for second strand priming (preferably at the end of the stop codon region as minor deletions or insertions may occur in the random region), etc. These regulatory nucleic acid sequences are operably linked to nucleic acids to be expressed. Nucleic acids are “operably linked” when it is placed into a functional relationship with another nucleic acid sequence. In addition, the selected regulatory nucleic acids, such as promoter sequences and translation initation sequences, will be appropriate to the host cell used, as is known to those skilled in the art.

When the retroviral vectors express fusion nucleic acids encoding a plurality of genes of interest, the separation sequence is operably linked to the first gene of interest and second gene of interest such that the fusion nucleic acid is capable of producing separate protein products of interest. Thus, in a preferred embodiment, the separation sequence is placed in between the first gene of interest and the second gene of interest. As will be appreciated by those skilled in the art, use of separation sequences based on protease recognition sites or Type 2A sequences requires that the fusion nucleic acid comprising the first gene of interest, separation sequence, and second gene of interest to be in-frame. By “in-frame” herein is meant that the fusion nucleic acid encodes a continuous single polypeptide comprising the protein encoded by the first gene of interest, protein encoded by the separation sequence, and protein encoded by the second gene of interest. Standard recombinant DNA techniques may be used for placing the components of the fusion nucleic to encode a contiguous single polypeptide. Peptide linkers may be added to the separation sequence to facilitate the separation reaction or limit structural interference of the separation sequence on the gene of interest (and vice versa). Preferred linkers are (Gly)n linkers, where n is 1 or more, with n being two, three, four, five or six, although linkers of 7-10 or amino acids are also possible.

As is appreciated by those in the art, use of IRES type sequences does not require the first gene of interest, separation sequence, and second gene of interest to be in frame since IRES elements function as internal translation initiation sites. Accordingly, fusion nucleic acids using IRES elements have the genes of interest arranged in a cistronic structure. That is, transcription of the fusion nucleic acid produces a cistronic mRNA that encodes both first gene of interest and second gene of interest with the IRES element controlling translation initiation of the downstream gene of interest. Alternatively, separate IRES sequences may control the upstream and downstream gene of interest.

Preferably the fusion nucleic acids are first cloned or constructed in a viral shuttle vector to produce a library of plasmids. A typical shuttle vector is pLNCX (Clontech, Palo Alto, Calif.). The resultant plasmid library can be amplified in E. coli, purified and introduced into retroviral packaging cell lines. Suitable retroviral packaging cell lines include, but are not limited to the Bing and BOSC23 cells lines (described in WO 94/19478; Soneoka, Y. et al. (1985) Nucleic Acids Res. 23: 628-33; Finer, M. H. et al. (1994) Blood 83: 43-50); Phoenix packaging lines such as PhiNX-ampho; 292T+gag pol and retrovirus envelope; PA 317; and other cell lines outlined in Markowitz, D. et al. (1998) Virology 167: 400-06 (see also Markowitz, D. et al. (1998) J. Virol. 63: 1120-24; Li, K. J. et al. (1996) Proc. Natl. Acad. Sci. USA 93: 11658-63; Kinsella, T. M. et al. (1996) Hum. Gene Ther. 7: 1405-13). Other packaging cell lines are commercially available, such as PT67 (Clontech, Palo Alto, Calif.). In a preferred embodiment, viruses are made by transient transfection of the packaging cell lines referenced above.

When the SIN vectors are based on lentiviruses, the vectors may be packaged by transfecting with plasmids encoding the necessary viral genes along with the vector construct (see Kafri, T. et al. (1997) Nat. Genet. 17: 314-317; Naldini, L. et al. (1996) Science 272: 263-67). In these transient transfection methods, the packaging plasmid constructs express Gag-pol, Tat, Rev, Nef, Vpr, Vpu and Vif proteins while the envelope plasmid constructs express the envelope protein, such as VSV-G, Env of MLV, or GaLV, to serve as the viral envelope. Cotransfection of lentivirus vectors with these plasmids results in packaging of the retroviral vector. Alternatively, lentivirus packaging cells lines that limit the cytotoxic effects of lentiviral proteins involved in viral packaging are used to generate and propagate the vector (Kafri, T. et al. (1999) J. Virol. 73: 576-84).

The resulting viruses can either be used directly or be used to infect another retroviral cell line for expansion of the library. In a preferred embodiment, the library of virus particles is used to transfect packaging cell lines disclosed herein to produce a primary viral library. By “primary viral library” herein is meant a library of virus particles comprising the fusion nucleic acids of the present invention. The production of the primary library is preferably done under conditions known in the art to reduce clone bias. The resulting primary viral library can be titred and stored, used directly to infect a target host cell line, or be used to infect another retroviral producer cell for “expansion” of the library. To obtain the secondary viral library, host cells are preferably infected with a multiplicity of infection (MOI) of 10. By “secondary viral library” herein is meant a library of retroviral particles expressing the fusion nucleic acids and candidate agents described herein.

Concentration of virus may be done as follows. Generally, retroviruses are titred by applying retrovirus containing supernatant onto indicator cells, for example NIH3T3 cells, and then measuring the percentage of cells expressing phenotypic consequences of infection. The concentration of virus is determined by multiplying the percentage of cell infected by the dilution factor involved, and taking into account the number of target cells available to obtain relative titre. If the retrovirus contains a reporter gene, such as lacZ, then infection, integration and expression of the recombinant virus is measured by histological staining for lacZ expression or by flow cytometry (i.e., FACS analysis). In general, retroviral titres generated from even the best of the producer cells do not exceed 10⁷ per ml unless concentrated, for example by centrifugation and ultrafiltration. However, flow through tranduction methods can provide up to a ten-fold higher infectivity by infecting cells on a porous membrane and allowing retrovirus supernatant to flow past the cells. This provides the capability of generating retroviral titres higher than those achieved by concentration (see Chuck, A. S. (1996) Hum. Gene Ther. 7: 743-50).

As will be appreciated by those in the art, these viral vectors or libraries of vectors are used to produce the transformed cells and transformed cellular libraries comprising fusion nucleic acids of SIN vectors. Generally, appropriate cells are infected with the virus, or in some cases transfected with retroviral vector in the presence of helper plasmids, to generate cells transformed with SIN vectors. Infection of the cells with virus is straightforward with the application of infection-enhancing reagent polybrene, which is a polycation that facilitates virus binding to the target cell. Infection can be optimized such that each cell generally expresses a single construct, using the ratio of virus particles to number of cells. Infection follows a Poisson distribution.

The phenotype produced by the stable integration of the retroviral vector provides a bases for identifying transformed cells. These phenotypes include expression of reporter genes, selection genes, or dominant phenotypes arising from expression of the retroviral fusion nucleic acid. For example, transformed cells may be identified based on stable expression of GFP or β-galatosidase reporter proteins expressed by the retroviral vector.

The type of cells used in the present invention can vary widely. Basically any mammalian cells may be used, including preferred cell types from mouse, rat, primate, and human cells. As is more fully described below, cell types implicated in a wide variety of disease conditions are particularly useful, so long as a suitable screen may be designed to allow the selection of transformed cells and cells that exhibit an altered phenotype as a consequence of the treating the cells with candidate agents, as described below. Of further use are cells types capable of displaying an inducible phenotype upon expression of a first and/or second gene of interest. These cells may be used to screen for candidate agents altering the particular induced phenotype.

The cell population or sample can contain a mixture of different cell types from either primary or secondary cultures although samples containing only a single cell type are preferred. For example, the sample can be from a cell line, particularly tumor cell lines, as outlined below. The cells may be in any cell phase, either synchronously or not, including M, G₁, S, and G₂. In a preferred embodiment, cells that are replicating or proliferating are used; this may allow the use of retroviral vectors for the introduction of candidate bioactive agents. Alternatively, non-replicating cells may be used in conjunction with a SIN vector capable of infecting non-dividing cells, such as lentivirus based retroviral vectors. Preferred cell types for use in the invention include, but are not limited to, mammalian cells, including animal (e.g., rodents, including mice, rats, hamsters and gerbils), primate, and human cells. Moreover, modifications of the system by pseudotyping allows most eukaryotic cells to be used, especially in higher eukaryotes (Morgan, R. A. et al. (1993) J. Virol. 67: 4712-21; Yang, Y. et al. (1995) Hum. Gene Ther. 6:1203-13).

Accordingly, suitable cell types include, but are not limited to, tumor cells of all types (particularly melanoma, myeloid leukemia, carcinomas of the lung, breast, ovaries, colon, kidney, prostate, pancreas, and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as hemopoietic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes.

Suitable cells also include known research cells, including, but not limited to, Jurkat T cells, NIH3T3 cells, CHO, Cos, etc. (see the ATCC cell line catalog, hereby expressly incorporated by reference).

In a preferred embodiment, the transformed cell comprises a single SIN vector comprising fusion nucleic acids. That is, each transformed cell comprises a single SIN vector. Generating a transformed cell comprising a single SIN vector is relatively straight forward and may be made by adjusting the multiplicity of infection (MOI) and detecting cells containing a single copy of the vector, for example by hybridization (e.g., Southern hybridization or in situ hybridization).

In another preferred embodiment, the transformed cell comprises a plurality or multiple SIN vectors. That is, each transformed cell comprises a plurality or multiple SIN vectors. By a “plurality” or “multiple” of SIN vectors herein is meant a transformed cell comprising two or more SIN vectors. In one preferred embodiment, the transformed cell comprises the same SIN vectors. This type of cell is desirable when higher levels of fusion nucleic acid expression are needed within the cell, for example in amplifying a reporter gene signal, inducing a cellular phenotype when expressing dominant phenotype proteins, and expressing candidate agents in the cell. In another preferred embodiment, the transformed cell comprises different SIN vectors. This type of cell is desirable, in part, for differentially regulating expression of fusion nucleic acids and for expressing different genes of interest.

Accordingly, in one preferred embodiment, the plurality of SIN vectors in the transformed cells comprise fusion nucleic acids comprising the same promoters. Use of the same promoter allows concerted regulation and expression of the fusion nucleic acids, thus providing uniform expression within the cell and throughout the cell population. The promoters may be constitutive or inducible. If inducible, a single inducer allows regulating expression of the plurality of SIN vectors.

In another preferred embodiment, the plurality of SIN vectors comprise fusion nucleic acids comprising different promoters. That is, the transformed cell comprises at least one SIN vector comprising a promoter and at least one SIN vector comprising a different promoter. Transformed cells containing fusion nucleic acids comprising different promoters allows for differentially regulating expression of the fusion nucleic acids and genes of interest for each type of SIN vector. In one aspect, the different promoters have differing transcriptional activities or promoter strengths such that the fusion nucleic acid of one SIN vector is expressed at levels higher than the fusion nucleic acid of another SIN vector within the transformed cell. By “transcriptional activity” or “promoter strength” herein is meant the level of trancriptional events promoted by the promoter. This allows fine regulation of the relative numbers of expressed fusion nucleic acids within the transformed cell.

In another aspect, the different promoters are differentially regulated. One promoter may be constitutive while another promoter is inducible. This arrangement allows continued expression of one fusion nucleic acid while allowing control over expression of the other fusion nucleic acid by use of inducing conditions. For example, the constitutive promoter may drive expression of a dominant effect protein while the inducible promoter regulates expression of candidate agents. Inducing expression of candidate agents provides a screen for bioactive agents that modulate effects of the dominantly acting protein. Alternatively, one promoter may be inducible with one inducer while the other promoter is inducible with a different inducer. This allows inducing one promoter under one condition and inducing the other promoter under another condition. In this way, only one of the promoters may be active or repressed at any time, or all promoters activated or repressed concomitantly. For example, at least one of the SIN vectors may comprise an IL-4 or IL-13 inducible IgEε promoter driving expression of a reporter gene (e.g., GFP) while at least one of the SIN vectors comprises a tetracycline regulated promoter controlling expression of candidate agents. If the tetracycline inducible transcription factor (e.g., tTA) is expressed in the transformed cell, expression of the candidate agents is inducible by removal of inducer (e.g., doxycycline). Thus, inducing both promoters provides a basis for identifying candidate agents affecting induction of the ε promoter by relevant cytokines.

In yet another preferred embodiment, the plurality of SIN vectors comprise fusion nucleic acids comprising the same gene of interest. Cell transformed with a plurality of SIN vectors expressing the same gene of interest allows for expressing elevated levels of the protein encoded by the gene of interest. For example, if the gene of interest encodes a reporter protein, signal amplification may be accomplished by expressing the identical reporter protein from a plurality of SIN vectors in the transformed cell.

In another preferred embodiment, the plurality of SIN vectors comprise fusion nucleic acids expressing different genes of interest, such as reporter genes, selection genes, dominant effect genes, etc. That is, at least one of the SIN vectors comprises a gene of interest and at least one of the SIN vectors comprises a different gene of interest. For example, if at least one of the SIN vectors expresses a reporter gene and at least one of the SIN vectors expresses a different reporter gene, the transformed cell is identifiable by two different basis, thus providing increased discrimination of cells expressing the different reporter genes. In addition, if the different genes of interest encode fusion proteins, they can be targeted to different cellular compartments by use of appropriate targeting signals. Thus, a cell transformed with a plurality of SIN vectors can express various combinations of different genes of interest.

In the present invention, any combination of SIN vectors comprising the fusion nucleic acids described herein may be used to generate transformed cells. Thus, in one aspect the transformed cell comprises SIN vectors comprising different promoters expressing the same gene of interest, thus providing the capability to adjust the copy number of the expressed fusion nucleic acid, especially if one promoter is inducible. In another aspect, the transformed cells comprises SIN vectors comprising same promoters expressing different genes of interest. This arrangement provides the capability of uniformly expressing the various fusion nucleic acids comprising different genes of interest, for example when different proteins encoded by the genes of interest interact, either directly or indirectly, to induce a particular phenotype on the transformed cell. In the present invention, these combinations also include SIN vectors comprising a first gene of interest, a separating sequence, and a second gene of interest.

In one preferred embodiment, the transformed cell comprises a SIN vector comprising a promoter, which drives expression of a gene of interest controlling the expression of a different SIN vector. That is, the transformed cell comprises a plurality of SIN vectors where at least one SIN vector comprises a promoter, which drives expression of a gene of interest that regulates expression of at least one of the SIN vectors comprising a different promoter driving expression of a different gene of interest. The regulation may be direct, for example where the gene of interest encodes a transcription factor acting directly on the different promoter, or the regulation may be indirect whereby the gene of interest regulates a cellular processes which regulates transcriptional activity of the different promoter. Thus, if the promoter of the SIN vector expressing the gene of interest is inducible, expression of the SIN vector comprising the different promoter and different gene of interest is rendered regulatable.

Transformed cells comprising a plurality or multiple SIN vectors is generated by methods well known in the art. When SIN vectors are the same, cells are infected at the appropriate multiplicity of infection (MOI) depending on the number of SIN vectors desired within a single cell. Transformed cells are selected based on expression of a detectable gene (e.g., reporter or selection gene) expressed by the SIN vector, and then examined for number of copies within the cell, for example by hybridization (e.g., Southern hybridization, in situ hybridization, etc.). When SIN vectors are different, the different SIN vectors express different detectable genes, i.e., different reporter or selection genes, which permits differentiating or distinguishing between the various SIN vectors. Transformed cells are identified based on expression of the repertoire of detectable genes expressed by the different SIN vectors. For example, if two different SIN vectors are used to transform a cell, one SIN vector expresses a GFP reporter gene and the other SIN vector expresses a hygromycin selection gene such that the transformed cells can be selected based on expression of both the reporter and selection gene.

The SIN vector expresses the detectable gene as the gene of interest or is expressed as the first or second gene of interest when separation sequences are used. Alternatively, an additional promoter different from the promoter used to express the gene of interest is used to drive expression of the detectable gene. That is, the fusion nucleic acid comprises at least two promoters where each promoter is operably linked to a gene of interest, one of which is a detectable gene used for identifying the appropriately transformed cells. This is useful where one of the promoter is inducible but inducing the promoter is not desirable when selecting for transformed cells, for example when expressing the gene of interest is detrimental to the cell.

In the present invention, cells transformed with a SIN vector or a plurality of SIN vectors are used to screen for candidate bioactive agents capable of producing an altered cellular phenotype. By candidate bioactive agent”, “candidate agent”, “candidate small molecules”, or “candidate expression products” (e.g., protein, oligopeptide, small organic molecule, polysaccharide, polynucleotide, etc.) or grammatical equivalents herein is meant an agent or expression product which may be tested for the ability to alter the phenotype of a cell.

Candidate bioactive agents encompass numerous chemical classes, though typically they are organic molecules, preferably small organic compounds having a molecular weight of more than 100 and less than about 2,500 daltons. Candidate agents comprise functional groups necessary for structural interaction with proteins, particularly hydrogen bonding, and typically include at least an amine, carbonly, hydroxyl, or carboxyl group, preferably at least two of them functional chemical groups. The candidate agents often comprise cyclical carbon or heterocyclic structures, and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are proteins, candidate drugs, and other small molecules.

Candidate agents are obtained from a wide variety of sources including libraries of synthetic or natural compounds. For example, numerous means are available for random and directed synthesis of a wide variety of organic compounds and biomolecules, including expression of randomized oligonucleotides (see for example, Gallop, M. A. et al. (1994) J. Med. Chem. 37: 1233-51; Gordon, E. M. et al. (1994) J. Med. Chem. 37:1385-401; Thompson, L. A. et al. (1996) Chem. Rev. 96: 555-600; Balkenhol, F. et al. (1996) Angew. Chem. Int. Ed. 35: 2288-337; and Gordon, E. M. et al. (1996) Acc. Chem. Res. 29: 444-54). Alternatively, libraries of natural compounds in the form of bacterial, fungal, plant and animal extracts are available or readily produced. Additionally, natural or synthetically produced libraries and compounds are readily modified through conventional chemical, physical, and biochemical means. Known pharmacological agents may be subjected to directed or random chemical modifications such as acylation, alkylation, esterification, and amidification to produce structural analogs.

The candidate agent can be pesticides, insecticides or environmental toxins; a chemical (including solvents, polymers, organic molecules, etc); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including prokaryotic and eukaryotic (including pathogenic cells), including mammalian tumor cells); viruses (including retroviruses, herpes viruses, adenoviruses, lentiviruses, etc.); and spores (e.g., fungal, bacterial, etc.).

One preferred embodiment of candidate agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus, “amino acid” or “peptide residue”, as used herein means both naturally occurring and synthetic amino acids. For example, homo-phenylalanine, citrulline, and norleucine are considered amino acids for the purposes of the invention. “Amino acids” also includes imino residues such as proline and hydroxyproline. The side chains may be either the (R) or (S) configuration. In the preferred embodiment, the amino acids are in the (S) or L configuration. If non-naturally occurring side chains are used, non-amino acid substituents may be used for example to prevent or retard in-vivo degradations. Proteins including non-naturally occurring amino acids may be synthesized or in some cases, made by recombinant techniques (see van Hest, J. C. et al. (1998) FEBS Lett. 428: 68-70 and Tang et al. (1999) Abstr. Pap. Am. Chem. S218: U138-U138 Part 2, both of which are expressly incorporated by reference herein).

In a preferred embodiment, the candidate bioactive agents are naturally occurring proteins or fragments of naturally occurring proteins. For example, cellular extracts containing proteins, or random or directed digests of proteinaceous cellular extracts, may be used. In this way, libraries of procaryotic and eukaryotic proteins may be made for screening in the systems described herein. Particularly preferred in this embodiment are libraries of bacterial, fungal, viral, and mammalian proteins, with the latter being preferred, and human proteins being especially preferred.

Candidate agents may encompass a variety of peptidic agents. These include, but are not limited to, (1) immunoglobulins, particularly IgEs, IgGs and IgMs, and particularly therapeutically or diagnostically relevant antibodies, including but not limited to, antibodies to human albumin, apolipoproteins (including apolipoprotein E), human chorionic gonadotropin, cortisol, a-fetoprotein, thyroxin, thyroid stimulating hormone (TSH), antithrombin, antibodies to pharmaceuticals (including antieptileptic drugs (phenytoin, primidone, carbariezepin, ethosuximide, valproic acid, and phenobarbitol), cardioactive drugs (digoxin, lidocaine, procainamide, and disopyramide), bronchodilators (theophylline), antibiotics (chloramphenicol, sulfonamides), antidepressants, immunosuppresants, abused drugs (amphetamine, methamphetamine, cannabinoids, cocaine and opiates) and antibodies to any number of viruses (including orthomyxoviruses, (e.g., influenza virus), paramyxoviruses (e.g., respiratory syncytial virus, mumps virus, measles virus), adenoviruses, rhinoviruses, coronaviruses, reoviruses, togaviruses (e.g., rubella virus), parvoviruses, poxviruses (e.g., variola virus, vaccinia virus), enteroviruses (e.g., poliovirus, coxsackievirus), hepatitis viruses (including A, B and C), herpesviruses (e.g., Herpes simplex virus, varicella-zoster virus, cytomegalovirus, Epstein-Barr virus), rotaviruses, Norwalk viruses, hantavirus, arenavirus, rhabdovirus (e.g., rabies virus), retroviruses (including HIV, HTLV-I and -II), papovaviruses (e.g., papillomavirus), polyomaviruses, and picornaviruses, and the like), and bacteria (including a wide variety of pathogenic and non-pathogenic prokaryotes of interest including Bacillus; Vibrio, e.g., V. cholerae; Escherichia, e.g., Enterotoxigenic E. coli, Shigella, e.g. S. dysenteriae; Salmonella, e.g., S. typhi; Mycobacterium e.g., M. tuberculosis, M. leprae; Clostridium, e.g., C. botulinum, C. tetani, C. difficile, C. perfringens; Cornyebacterium, e.g., C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lamblia Y. pestis, Pseudomonas, e.g. P. aeruginosa, P. putida; Chlamydia, e.g., C. trachomatis; Bordetella, e.g., B. pertussis; Treponema, e.g., T. palladium; and the like); (2) enzymes (and other proteins), including but not limited to, enzymes used as indicators of or treatment for heart disease, including creatine kinase, lactate dehydrogenase, aspartate amino transferase, troponin T, myoglobin, fibrinogen, cholesterol, triglycerides, thrombin, tissue plasminogen activator (tPA); pancreatic disease indicators including amylase, lipase, chymotrypsin and trypsin; liver function enzymes and proteins including cholinesterase, bilirubin, and alkaline phosphatase; aldolase, prostatic acid phosphatase, terminal deoxynucleotidyl transferase, and bacterial and viral enzymes such as HIV protease; (3) hormones and cytokines (many of which serve as ligands for cellular receptors) such as erythropoietin (EPO), thrombopoietin (TPO), the interleukins (including IL-1 through IL-17), insulin, insulin-like growth factors (including IGF-1 and -2), epidermal growth factor (EGF), transforming growth factors (including TGF-α and TGF-β), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cortisol, estradiol, follicle stimulating hormone (FSH), thyroid-stimulating hormone (TSH), luteinizing hormone (LH), progesterone, testosterone,; and (4) other proteins (including α-fetoprotein, carcinoembryonic antigen CEA).

In a preferred embodiment, the candidate bioactive agents are peptides of from about 5 to about 30 amino acids, with from about 5 to about 20 amino acids being preferred, and from about 7 to about 15 being particularly preferred. These peptides may be digests of naturally occurring proteins, as described above, or random or biased random peptides and peptide analogs either chemically synthesized or encoded by candidate nucleic acids. By “randomized” or grammatical equivalents herein is meant that each nucleic acid and peptide consists of essentially random nucleotides and amino acids, respectively. Generally, since these random peptides (or nucleic acids, discussed below) are chemically synthesized, they may incorporate any amino acid or nucleotide at any position. The synthetic process can be designed to generate randomized proteins or nucleic acids to allow the formation of all or most of the possible combinations over the length of the sequence, thus forming a library of randomized candidate bioactive proteinaceous agents.

In one embodiment, the library is fully randomized, with no sequence preference or constants at any position. In a preferred embodiment, the library is biased. That is, some positions within the sequence are either held constant or are selected from a limited number of possibilities. For example, in a preferred embodiment, the nucleotides or amino acid residues are randomized within a defined class, for example hydrophobic amino acids, hydrophilic residues, sterically biased (either small or large) residues, or are amino acid residues for crosslinking (e.g., cysteines) or phosphorylation sites (i.e., serines, threonines, tyrosines, or histidines).

In a preferred embodiment, the bias is toward peptides or nucleic acids that interact with known classes of molecules. For example, it is known that much of intracellular signaling is carried out by short regions of polypeptide interacting with other polypeptide regions of other proteins, such as the interaction domains described above. Another example of interaction domain is a short region from the HIV-1 envelope cytoplasmic domain that has been previously shown to block the action of cellular calmodulin. Regions of the Fas cytoplasmic domain, which shows homology to the mastoparn toxin from Wasps, can be limited to a short peptide region with death inducing apoptotic or G protein inducing functions. Magainin, a natural peptide derived from Xenopus, can have potent anti-tumor and anti-microbial activity. Short peptide fragments of a protein kinase C isozyme (β-PKC) have been shown to block nuclear translocation of PKC in Xenopus oocytes following stimulation. In addition, short SH-3 target proteins have been used as pseudosubstrates for specific binding to SH-3 proteins. This is of course a short list of available peptides with biological activity, as the literature is dense in this area. Thus, there is much precedent for the potential of small peptides to have activity on intracellular signaling cascades. In addition, agonists and antagonists of any number of molecules may be used as the basis of biased randomization of candidate bioactive agents as well.

Thus, a number of molecules or protein domains are suitable as starting points for generating biased candidate agents. A large number of small molecule domains are known that confer common function, structure or affinity. These include protein-protein interaction domains and nucleic acid interaction domains described above. As is appreciated by those in the art, while variations of these protein-protein or protein-nucleic acid domains may have weak amino acid homology, the variants may have strong structural homology.

In another preferred embodiment, the candidate agents are nucleic acids. By “nucleic acid” or “oligonucleotide” or grammatical equivalents herein is meant at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage, S. L. et al. (1993) Tetrahedron 49: 1925-63 and references therein; Letsinger, R. L. et al. (1970) J. Org. Chem. 35: 3800-03; Sprinzl, M. et al. (1977) Eur. J. Biochem. 81: 579-89; Letsinger, R. L. et al. (1986) Nucleic Acids Res. 14: 3487-99; Sawai et al. (1984) Chem. Left. 805; Letsinger, R. L. et al. (1988) J. Am. Chem. Soc. 110: 4470; and Pauwels et al. (1986) Chemica Scripta 26:141-49), phosphorothioate (Mag, M. et al. (1991) Nucleic Acids Res. 19: 1437-41; and U.S. Pat. No. 5,644,048), phosphorodithioate (Briu et al. (1989) J. Am. Chem. Soc. 111: 2321), O-methylphophoroamidite linkages (see Eckstein, Oligonucleotides and Analogues: A Practical Approach, Oxford University Press, 1991), and peptide nucleic acid backbones and linkages (Egholm, M. (1992) Am. Chem. Soc. 114:1895-97; Meier et al. (1992) Chem. Int. Ed. Engl. 31:1008; Egholm, M (1993) Nature 365: 566-68; Carlsson, C. et al. (1996) Nature 380: 207, all of which are incorporated by reference). Other analog nucleic acids include those with positive backbones (Dempcy, R. O. et al. (1995) Proc. Natl. Acad. Sci. USA 92: 6097-101); non-ionic backbones (U.S. Pat. Nos. 5,386,023, 5,637,684, 5,602,240, 5,216,141 and 4,469,863; Kiedrowshi et al. (1991) Angew. Chem. Intl. Ed. English 30: 423; Letsinger, R. L. et al. (1988) J. Am. Chem. Soc. 110: 4470; Letsinger, R. L. et al. (1994) Nucleoside & Nucleotide 13: 1597; Chapters 2 and 3, ASC Symposium Series 580, “Carbohydrate Modifications in Antisense Research”, Ed. Y.S. Sanghui and P. Dan Cook; Mesmaeker et al. (1994) Bioorganic & Medicinal Chem. Lett. 4: 395; Jeffs et al. (1994) J. Biomolecular NMR 34: 17; (1996) Tetrahedron Lett. 37: 743) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al. (1995) Chem. Soc. Rev. 169-76). Several nucleic acid analogs are described in Rawls, C & E News Jun. 2, 1997 page 35. All of these references are hereby expressly incorporated by reference. These modifications of the ribose-phosphate backbone may be done to facilitate the addition of additional moieties, such as labels, or to increase the stability and half-life of such molecules in physiological environments. In addition, mixtures of different nucleic acid analogs, and mixtures of naturally occurring nucleic acids and analogs may be made. The nucleic acids may be single stranded or double stranded, as specified, or contain portions of both double stranded or single stranded sequence. The nucleic acid may be DNA, both genomic and cDNA, RNA or hybrid, where the nucleic acid contains any combination of deoxyribo- and ribonucleotides, and any combination of bases, including uracil, adenine, thymine, cytosine, guanine, xanthine hypoxanthine, isocytosine, isoguanine, etc., although generally occurring bases are preferred. In a preferred embodiment, the candidate nucleic acids comprise cDNAs, including cDNA libraries, or fragments of cDNAs. The cDNAs can be derived from any number of different cells and include cDNAs generated from eucaryotic and procaryotic cells, viruses, cells infected with viruses or other pathogens, genetically altered cells, cells with defective cellular processes, etc. Preferred embodiments include cDNAs made from different individuals, such as different patients, particularly human patients. The cDNAs may be complete libraries or partial libraries. Furthermore, the candidate nucleic acids can be derived from a single cDNA source or multiple sources; that is, cDNA from multiple cell types, multiple individuals or multiple pathogens can be combined in a screen. In other aspects, the cDNA may encode specific domains, such as signaling domains, protein interaction domains, membrane binding domains, targeting domains, etc. The cDNAs may utilize entire cDNA constructs or fractionated constructs, including random or targeted fractionation. Suitable fractionation techniques include enzymatic (e.g., DNase I, restriction nucleases etc.), chemical, or mechanical fractionation (e.g., sonicated or sheared). Also useful for the present invention are cDNA libraries enriched for a specific class of proteins, such as type I membrane proteins (Tashiro, K. et al. (1993) Science 261: 600-03) and membrane proteins (Kopczynski, C.C. (1998) Proc. Natl. Acad. Sci. USA 95: 9973-78). Additionally, subtracted cDNA libraries in which genes preferentially or exclusively expressed in particular cells, tissues, or developmental phases are enriched. Methods for making subtracted cDNA libraries are well known in the art (see Diatchenko, L. et al. (1999) Methods Enzymol. 303: 349-80; von Stein, O. D. et al. (1997) Nucleic Acids Res. 13: 2598-602: Carcinci, P. (2000) Genome Res. 10: 1431-32). Accordingly, a cDNA library may be a complete cDNA library from a cell, a partial library, an enriched library from one or more cell types, or a constructed library with certain cDNAs being removed to from a library. In another preferred embodiment, the candidate nucleic acids comprise libraries of genomic nucleic acids, which includes organellar nucleic acids. As elaborated above for cDNAs, the genomic nucleic acids may be derived from any number of different cells, including genomic nucleic acids of eukaryotes, prokaryotes, or viruses. They may be from normal cells or cells defective in cellular processes, such as tumor suppression, cell cycle control, or cell surface adhesion. Moreover, the genomic nucleic acids may be obtained from cells infected with pathogenic organisms, for example cells infected with viruses or bacteria. The genomic nucleic acids comprise entire genomic nucleic acid constructs or fractionated constructs, including random or targeted fractionation as described above. Generally, for genomic nucleic acids and cDNAs, the candidate nucleic acids may range from nucleic acid lengths capable of encoding proteins of twenty to thousands of amino acid residues, with from about 50-1000 being preferred and from about 100-500 being especially preferred. In addition, candidate agents comprising cDNA or genomic nucleic acids may also be subsequently mutated using known techniques (e.g., exposure to mutagens, error prone PCR, error prone transcription, combinatorial splicing (e.g., cre-lox recombination) to generate novel nucleic acid sequences (or protein sequences). In this way libraries of procaryotic and eukaryotic nucleic acids may be made for screening in the systems described herein. Particularly preferred in the embodiments are libraries of bacterial, fungal, viral and mammalian nucleic acids, with the latter being preferred, and human nucleic acids being especially preferred.

In another preferred embodiment, the candidate nucleic acids comprise libraries of random nucleic acids. Generally, the random nucleic acids are fully randomized or they are biased in their randomization, e.g. in nucleotide/residue frequency generally or per position. As defined above, by “randomized” or grammatical equivalents herein is meant that each nucleic acid consists essentially of random nucleotides. Since the candidate nucleic acids are chemically synthesized, they may incorporate any nucleotide at any position. In the expressed random nucleic acid, at least 10, preferably at least 12, more preferably at least 15, most preferably at least 21 nucleotide positions need to be randomized. The candidate nucleic acids may also comprise nucleic acid analogs as described above.

For candidate nucleic acids encoding peptides, the candidate nucleic acids generally contain cloning sites which are placed to allow in-frame expression of the randomized peptides, and any fusion partners, if present, such as presentation structures. For example, when presentation structures are used, the presentation structure will generally contain the initiating ATG as part of the parent vector. For candidate agents comprising RNAs, in addition to chemically synthesized RNA nucleic acids, the candidate nucleic acids may be expressed from vectors, including retroviral vectors. Thus, when the RNAs are expressed, vectors expressing the candidate nucleic acids may be constructed with an internal promoter (e.g., CMV promoter), tRNA promoter, cell specific promoter, or hybrid promoters designed for immediate and appropriate expression of the RNA structure at the initiation site of RNA synthesis. For retroviral vectors, the RNA may be expressed anti-sense to the direction of retroviral synthesis and is terminated as known, for example with an orientation specific terminator sequences. Interference from upstream transcription is minimized in the target cell by using the SIN vectors described herein.

When the nucleic acids are expressed in the cells, they may or may not encode a protein as described herein. Thus, included within the candidate nucleic acids of the present invention are RNAs capable of producing an altered phenotype. In this regard, the nucleic acid may be an antisense RNA directed towards a complementary target nucleic acid, RNAs capable of catalyzing cleavage of target nucleic acids in a sequence specific manner, preferably in the form of ribozymes (e.g., hammerhead ribozymes, hairpin ribozymes, and hepatitis delta virus ribozymes), and double stranded RNA capable of inducing RNA interference or RNAi, as described above.

In a preferred embodiment, a library of candidate bioactive agents are used. Preferably, the library should provide a sufficiently structurally diverse population of randomized expression products to effect a probabilistically sufficient range to provide one or more peptide products which has the desired properties such as binding to protein interaction domains or producing a desired cellular response. For example, in the case of libraries of random peptides, a library must be large enough so that at least one of its members will have a structure that gives it affinity for some molecule, protein or other factor whose activity is involved in some cellular response, such as signal transduction. Although it is difficult to gauge the required absolute size of an interaction library, nature provides a hint with the immune response: a diversity of 10⁷-10⁸ different antibodies provides at least one combination with sufficient affinity to interact with most potential antigens faced by an organism.

Published in vitro selection techniques have also shown that a library size of about 10⁶ to 10⁸ is sufficient to find structures with affinity for the target. A library of all combinations of a peptide 7-20 amino acids in length, such as proposed here for expression in retroviruses, has the potential to code for 20⁷ (10⁹) to 20²⁰. Thus with libraries of 10⁷ to 10⁸ per ml of retroviral particles the present methods allow a “working” subset of a theoretically complete interaction library for 7 amino acids, ad a subset of shapes for the 20²⁰ library. Thus in a preferred embodiment, at least 10⁶, preferably at least 10⁷, more preferably at least 10⁸, and most preferably at least 10⁹ different expression products are simultaneously analyzed in the subject methods. Preferred methods maximize library size and diversity.

The candidate bioactive agents are combined, added to, or contacted with a cell or population of cells or plurality of cells. By “population of cells” or “plurality of cells” herein is meant at least two cells, with at least about 10⁵ being preferred, at least about 10⁶ being particularly preferred, and at least about 10^{7, 10}⁸, and 10⁹ being especially preferred.

The candidate agents and the cells are combined. As will be appreciated by those in the art, this may be accomplished in any number of ways, including adding the candidate agents to the surface of the cells, to the media containing the cells, or to a surface on which the cells grow or contact. The candidate agents and cells may be combined by adding the agents into the cells, for example by using vectors that will introduce agents into the cells, especially when the candidate agents are nucleic acids or proteins.

In a preferred embodiment, the candidate agents are either nucleic acids or proteins that are introduced into the cells to screen for candidate agents capable of altering the phenotype of a cell. By “introduced into” or grammatical equivalents herein is meant that the nucleic acids enter the cells in a manner suitable for subsequent expression of the nucleic acid. The method of introduction is largely dictated by the targeted cell type, discussed below. Exemplary methods include CaPO₄ transfection, DEAE dextran transfection, liposome fusion, lipofectin®), electroporation, viral infection, biolistic particle bombardment etc. The candidate nucleic acids may exist either transiently or stably in the cytoplasm or stably integrate into the genome of the host cell (i.e., by retroviral integration). As many pharmaceutically important screens require human or model mammalian cell targets, retroviral vectors capable of transfecting such targets are preferred.

In a preferred embodiment, the candidate bioactive agents are either nucleic acids or proteins (proteins in this context includes proteins, oligopeptides, and peptides) that are expressed in the host cells using vectors, including viral vectors. The choice of the vector, preferably a viral vector, will depend on the cell type. When cells are replicating, retroviral vectors are used. When the cells are not replicating, for example when arrested in one of the growth phases, viral vectors capable of infecting non-dividing cells, including lentiviral and adenoviral vectors, are used to express the nucleic acids and proteins.

In a preferred embodiment, the candidate bioactive agents are either nucleic acids or proteins that are introduced into the host cells using retroviral vectors, as is generally outlined in PCT US 97/01019 and PCT US97/01048, both of which are expressly incorporated by reference. Generally, a library is generated using a retroviral vector backbone. For generating a random nucleic acid or peptide library, standard oligonucleotide synthesis is done to generate the nucleic acids. After synthesizing the nucleic acid library, the library is cloned into a first primer, which serves as a cassette for insertion into the retroviral construct. The first primer generally contains additional elements, including for example, the required regulatory sequences (e.g., translation, transcription, promoters, etc.) fusion partners, restriction endonuclease sites, stop codons, regions of complementarity for second strand priming.

A second primer is then added, which generally consists of some or all of the complementarity region to prime the first primer and optional sequences necessary to a second unique restriction site for purposes of subcloning. Extension with DNA polymerase results in double stranded oligonucleotides, which are then cleaved with appropriate restriction endonucleases and subcloned into the target retroviral vectors.

When the candidate agents are cDNAs or genomic DNAs, these nucleic acids are inserted into the retroviral vector by methods well known in the art. The DNAs may be inserted unidirectionally or randomly using appropriate adaptor sequences and vector restriction sites.

Any number of suitable retroviral vectors may be used. In one aspect, preferred vectors include those based on murine stem cell virus (MSCV) (Hawley, et al. (1994) Gene Therapy 1: 136), a modified MFG virus (Reivere et al. (1995) Genetics 92: 6733), pBABE, and others described above. Well suited retroviral transfection systems are described in Mann et al, supra; Pear et al. (1993) Proc. Natl. Acad. Sci. USA 90: 8392-96; Kitamura, et al. Human Gene Ther. 7: 1405-1413; Hofmann, et al Proc. Natl. Acad. Sci. USA 93: 5185-90; Choate et (1996) Human Gene Ther 7: 2247; WO 94/19478; PCT US97/01019, and references cited therein, all of which are incorporated by reference.

In one preferred embodiment, the retroviral vectors used to introduce candidate agents comprise the SIN vectors described herein. Thus, the SIN vectors comprising a promoter and a gene of interest, as described above, may be used to express the candidate nucleic acids, including candidate nucleic acids encoding peptides and proteins. A plurality of SIN vectors expressing candidate nucleic acids may be present in a cell, thus allowing expression of novel combinations of candidate nucleic acids and candidate peptides within a single cell. In another aspect, the candidate nucleic acids are introduced as SIN vectors comprising a promoter, a first gene of interest, a separation sequence, and a second gene of interest. In these constructs, at least one of the genes of interest comprises the fusion nucleic acid comprising the candidate nucleic acids. The use of a separation sequence and a reporter/selection gene allows identification of cells expressing the candidate nucleic acids and candidate peptides. In another aspect, the first and second genes of interest comprise nucleic acids encoding different candidate agents, thus permitting expression of multiple candidate agents within a single cell. As above, expressing multiple candidate agents allows for screening of novel combinations of candidate agents within a single cell and, in addition, permits more rapid screening of libraries of candidate agents.

Accordingly, the transformed cells of the present invention may comprise cellular libraries transformed with libraries of SIN vectors comprising fusion nucleic acids expressing candidate agents. These cellular libraries may comprise libraries of SIN vectors expressing candidate nucleic acids, candidate peptides, cDNAs, or genomic DNAs, as described above.

The retroviral vectors used to introduce candidate agents may include inducible, constitutive, or cell specific promoters for the expression of the candidate agents. For example, there are situations wherein it is necessary to induce peptide expression only during certain phases of the selection process, such as during particular periods of the cell cycle. A large number of constitutive, inducible, and cell specific promoters are well known, and may be used to regulate expression of the candidate agents.

In a preferred embodiment, the bioactive candidate agents are linked to a fusion partner, as described above. In one aspect, combinations of fusion partners are used. Any number of combinations of presentation structures, targeting sequences, rescue sequences, and stability sequences may be used with or without linker sequences.

Candidate agents, which include these components, may be used to generate a library of fusion nucleic acids where each member contains a different nucleotide sequence, for example a random sequence, that may encode a different peptide sequence. The ligation products are then transformed into bacteria, such as E. coli, and DNA is prepared from the resulting library as generally outlined in Kitamura, T. (1995) Proc. Natl. Acad. Sci. USA 92: 9146-50.

In a preferred embodiment, when the candidate agent is introduced to the cells using viral vectors, the candidate peptide agent is linked to a detectable molecule, and the methods of the invention include at least one expression assay. An expression assay is an assay that allows the determination of whether a candidate bioactive agent has been expressed, i.e., whether a candidate peptide agent is present in the cell. The detectable molecule may comprise reporter and selection genes as described herein. In one preferred embodiment, the detectable molecule is distinguishable from that expressed by the fusion nucleic acid expressing the genes of interest. By linking the expression of a candidate agent to the expression of a detectable molecule such as a label, the presence or absence of the candidate peptide agent may be determined. Accordingly, in this embodiment, the candidate agent is operably linked to a detectable molecule. Generally, this is done by creating a fusion nucleic acid. The fusion nucleic acid comprises a first nucleic acid expressing the candidate bioactive agent (which can include fusion partners, as outlined above), and a second nucleic acid expressing a detectable molecule. The fusion nucleic acid may use one promoter for the first nucleic and a second promoter for the second nucleic acid to produce separate nucleic acids comprising a candidate nucleic acid, which may or may not encode a protein, and the detectable molecule. This may also be accomplished by using a fusion nucleic acid having a separation sequence, as described herein, to express separate candidate bioactive agent and detectable molecule. Alternatively, the candidate peptide is fused directly to the detectable molecule (e.g., GPF), with or without linker sequences, to produce a fusion protein (see U.S. Pat. No. 6,180,343, hereby expressly incorporated by reference). As used herein, the terms “first” and “second” are not meant to confer an orientation of the sequences with respect to 5′-3′ orientation of the fusion nucleic acid. For example, assuming a 5′-3′ orientation of the fusion sequence, the first nucleic acid may be located either 5′ to the second nucleic acid, or 3′ to the second nucleic acid. Preferred detectable molecules in this embodiment include, but are not limited to, various fluorescent proteins and their variants, including A. Victoria GFP, Renilla muelleri GFP, Renilla reniformis GFP, Ptilosarcus gurneyi GFP, YFP, BFP, RFP, Anemonia majano fluorescent protein, Zoanthus fluorescent proteins, Discosoma fluorescent proteins, and Clavularia fluorescent proteins.

In general, the candidate agents are added to the cells (either extracellularly or intracellularly, as outlined above) under reaction conditions that favor agent-target interactions. Generally, this will be physiological conditions. Incubations may be performed at any temperature which facilitates optimal activity, typically between 4 and 40° C. Incubation periods are selected for optimum activity, but may also be optimized to facilitate rapid high throughput screening. Typically between 0.1 and 24 hr or up to 72 hrs will be sufficient. Excess reagent is generally removed or washed away.

A variety of other reagents may be included in the assays. These include reagents like salts, neutral proteins (e.g., albumin), detergents, etc. which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for detection. Washing or rinsing the cells will be done as will be appreciated by those in the art at different times, and may include the use of filtration and centrifugation. When second labeling moieties (also referred to herein as “secondary labels”) are used, they are preferably added after excess non-bound target molecules are removed in order to reduce non-specific binding. However, under some circumstances, all the components may be added simultaneously.

As will be appreciated by those in the art, the type of cells used in the present invention can vary widely. Basically, the screen may use any mammalian cells in which the library of retroviral vectors of the present invention are made. Particularly preferred are cells from mouse, rat, primate and human cells, although as will be appreciated by those in the art, modifications of the system by pseudotyping allows all eukaryotic cells to be used, preferably higher eukaryotes (Morgan, R. A. et al. (1993) J. Virol. 67: 4712-21; Yang, Y. et al. (1995) Hum. Gene Ther. 6: 1203-13).

As is more fully described below, a screen is set up such that the cells exhibit a selectable phenotype in the presence of a candidate agent. Cell types implicated in a wide variety of disease conditions are particularly useful, so long as a suitable screen may be designed to allow the selection of cells that exhibit an altered phenotype as a consequence of the presence of a candidate bioactive agent within the cell.

Accordingly, suitable cell types include, but are not limited to, tumor cells of all types (particularly melanoma, myeloid leukemia, carcinomas of the lung, breast, ovaries, colon, kidney, prostate, pancreas, and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as hemopoietic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, etc. (see the ATCC cell line catalog, hereby expressly incorporated by reference).

In a preferred embodiment, a first plurality of cells is screened. That is, the cells into which the candidate nucleic acids are introduced are screened for an altered phenotype. Thus, in this embodiment, the effect of the bioactive candidate agent is seen in the same cells in which it is made;

i.e., an autocrine effect.

By a “plurality of cells” herein is meant roughly from about 10³ cells to 10⁸ or 10⁹, with from 10⁶ to 10⁸ being preferred. This plurality of cells comprises a cellular library, wherein generally each cell within the library contains a member of the retroviral molecular library, i.e., a different candidate nucleic acid, although as will be appreciated by those in the art, some cells within the library may not contain a retrovirus, and some may contain more than one. When methods other than retroviral infection are used to introduce the candidate nucleic acids into a plurality of cells, the distribution of candidate nucleic acids within the individual cell members of the cellular library may vary widely, as it is generally difficult to control the number of nucleic acids which enter a cell during electroporation, transfection etc.

In a preferred embodiment, the candidate nucleic acids are introduced into a first plurality of cells, and the effect of the candidate bioactive agents is screened in a second or third plurality of cells, different from the first plurality of cells, i.e., generally a different cell type. That is, the effect of the bioactive agents is due to an extracellular effect on a second cell; i.e., an endocrine or paracrine effect. This is done using standard techniques. The first plurality of cells may be grown in or on one media, and the media is allowed to touch a second plurality of cells, and the effect measured. Alternatively, there may be direct contact between the cells. Thus, contacting is functional contact, and includes both direct and indirect. In this embodiment, the first plurality of cells may or may not be screened.

If necessary, the cells are treated to conditions suitable for expression of the candidate nucleic acid; for example, when inducible promoter are used to express the candidate agents. Expression of the candidate agents results in functional contact of the candidate agent and the cell.

The plurality of cells is then screened, as is more fully outlined below, for a cell exhibiting an altered phenotype. The altered phenotype is due to the presence of a candidate bioactive agent. By “altered phenotype” or “changed physiology” or other grammatical equivalents herein is meant that the phenotype of the cell is altered in some way, preferably in some detectable and/or measurable way. As will be appreciated in the art, a strength of the present invention is the wide variety of cell types and potential phenotypic changes which may be tested using the present methods. Accordingly, any phenotypic change which may be observed, detected, or measured may be the basis of the screening methods herein. Suitable phenotypic changes include, but are not limited to: gross physical changes such as changes in cell morphology, cell growth, cell viability, adhesion to substrates or other cells, and cellular density; changes in the expression of one or more RNAs, proteins, lipids, hormones, cytokines, or other molecules; changes in the equilibrium state (i.e., half-life) or one or more RNAs, proteins, lipids, hormones, cytokines, or other molecules; changes in the localization of one or more RNAs, proteins, lipids, hormones, cytokines, or other molecules; changes in the bioactivity or specific activity of one or more RNAs, proteins, lipids, hormones, cytokines, receptors, or other molecules; changes in the secretion of ions, cytokines, hormones, growth factors, or other molecules; alterations in cellular membrane potentials, polarization, integrity or transport; changes in infectivity, susceptibility, latency, adhesion, and uptake of viruses and bacterial pathogens; etc. By “capable of altering the phenotype” herein is meant that the candidate agent can change the phenotype of the cell in some detectable and/or measurable way.

The altered phenotype may be detected in a wide variety of ways, as is described more fully below, and will generally depend and correspond to the phenotype that is being changed. Generally, the changed phenotype is detected using, for example: microscopic analysis of cell morphology; standard cell viability assays, including both increased cell death and increased cell viability, for example, cells that are now resistant to cell death via virus, bacteria, or bacterial or synthetic toxins; standard labeling assays such as fluorometric indicator assays for the presence or level of a particular cell or molecule, including FACS or other dye staining techniques; biochemical detection of the expression of target compounds after killing the cells; etc. In some cases, as is more fully described herein, the altered phenotype is detected in the cell in which the randomized nucleic acid was introduced; in other embodiments, the altered phenotype is detected in a second cell which is responding to some molecular signal from the first cell.

In a preferred embodiment, once a cell with an altered phenotype is detected, the cell is isolated from the plurality which do not have altered phenotypes. Isolation of the altered cell may be done in any number of ways, as is known in the art, and will in some instances depend on the assay or screen. Suitable isolation techniques include, but are not limited to, FACS; lysis selection using complement; cell cloning; scanning by Fluorimager; expression of a “survival” protein; induced expression of a cell surface protein or other molecule that can be rendered fluorescent or taggable for physical isolation; expression of an enzyme that changes a non-fluorescent molecule to a fluorescent one; overgrowth against a background of no or slow growth; death of cells and isolation of DNA or other cell vitality indicator dyes; etc.

In a preferred embodiment, the candidate nucleic acid and/or the bioactive agent is isolated from the positive cell. In one aspect, primers complementary to DNA regions common to the retroviral constructs, or to specific components of the library such as a rescue sequence, as described above, are used to “rescue” the subject sequence. Alternatively, the bioactive candidate agent is isolated using a rescue sequence. Thus, for example, rescue sequences comprising epitope tags or purification sequences may be used to pull out the bioactive candidate agent, using immunoprecipitation or affinity columns. In some instances, as is outlined below, this may also pull out the primary target molecule if there is a sufficiently strong binding interaction between the bioactive agent and the target molecule. Alternatively, the peptide may be detected using mass spectroscopy.

Once rescued, the sequence of the candidate agent and/or bioactive nucleic acid is determined. This information can then be used in a number of ways.

In a preferred embodiment, the candidate agent is resynthesized and reintroduced into the target cells, to verify the effect. This may be done using retroviruses, or alternatively using fusions to the HIV-1 Tat protein, and analogs and related proteins, which allows very high uptake into target cells (see for example, Fawell, S. et al.(1994) Proc. Natl. Acad. Sci. USA 91: 664-68; Frankel, A. D. et al.(1988) Cell 55: 1189-93; Savion, N. et al. (1981)J. Biol. Chem. 256: 1149-54; Derossi, D. et al. (1994)J. Biol. Chem. 269:10444-50; and Baldin, V. et al. (1990) EMBO J. 9: 1511-17, all of which are incorporated by reference.

In a preferred embodiment, the sequence of a candidate agent is used to generate more candidate bioactive agents. For example, the sequence of the candidate agent may be the basis of a second round of (biased) randomization, to develop other candidate agents with increased or altered activities. Alternatively, the second round of randomization may change the affinity of the candidate agent.

Furthermore, it may be desirable to put the identified random region of the candidate agent into other presentation structures, or to alter the sequence of the constant region of the presentation structure, to alter the conformation/shape of the candidate agent. It may also be desirable to “walk” around a potential binding site, in a manner similar to the mutagenesis of a binding pocket, by keeping one end of the ligand region constant and randomizing the other end to shift the binding of the peptide around.

In a preferred embodiment, either the candidate agent or the candidate nucleic acid encoding it is used to identify target molecules. As will be appreciated by those in the art, there may be primary target molecules, to which the candidate agent binds or acts upon directly, and there may be secondary target molecules, which are part of the signaling pathway affected by the bioactive agent; these might be termed “validated targets”.

In a preferred embodiment, the bioactive agent is used to pull out target molecules. For example, as outlined herein, if the target molecules are proteins, the use of epitope tags or purification sequences can allow the purification of primary target molecules via biochemical means (co-immunoprecipitation, affinity columns, etc.). Alternatively, the peptide, when expressed in bacteria and purified, can be used as a probe against a bacterial cDNA expression library made from mRNA of the target cell type. Alternatively, peptides can be used as “bait” in either yeast or mammalian two or three hybrid systems. Such interaction cloning approaches have been very useful in isolating DNA-binding proteins and protein-protein interacting components. The peptide(s) can be combined with other pharmacologic activators to study the epistatic relationships of signal transduction pathways in question. It is also possible to synthetically prepare labeled peptide candidate agent and use it to screen a cDNA library expressed in bacteriophage for those expressed cDNAs which bind the peptide. Furthermore, it is also possible that one could use cDNA cloning via retroviral libraries to “complement” the effect induced by the peptide. In such a strategy, the peptide would be required to be stochiometrically titrating away some important factor for a specific signaling pathway. If this molecule or activity is replenished by over-expression of a cDNA from a cDNA library, then one can clone the target. Similarly, cDNAs cloned by any of the above yeast or bacteriophage systems can be reintroduced to mammalian cells in this manner to confirm that they act to complement function in the system the peptide acts upon.

Once primary target molecules have been identified, secondary target molecules may be identified in the same manner, using the primary target as the “bait”. In this manner, signaling pathways may be elucidated. Similarly, bioactive agents specific for secondary target molecules may also be discovered to identify a number of bioactive agents acting on a single pathway, for example for purposes of combination therapies.

The methods of the present invention may be useful for screening a large number of cell types under a wide variety of conditions. Generally, the host cells are cells are involved in disease states, and they are tested or screened under conditions that normally result in undesirable consequences on the cells. When a suitable bioactive candidate agent is found, the undesirable effect may be reduced or eliminated. Alternatively, normally desirable consequences may be reduced or eliminated, with an eye towards elucidating the cellular mechanisms associated with the disease state or signaling pathway.

Accordingly, the compositions and methods described herein are useful in a variety of applications. In one preferred embodiment, the SIN retroviral constructs are used to screen for modulators of promoter activity. By “modulation” of promoter activity herein is meant increase or decrease in transcription of nucleic acid regulated by the promoter of interest. A variety of promoters are amenable to analysis. Example of relevant promoters are IL-4 inducible ε promoter, IgH promoter, NF-kβ regulated promoters, APC/β-catenin regulated promoters, myc regulated promoters, and promoters regulating HIV viral gene expression and cell cycle genes. Preferred are promoters regulating expression of signal transduction proteins, cell cycle regulatory proteins, oncogenes, or promoters which are themselves regulated by signal transduction pathways, cell cycle regulators, or other aspects of cell regulatory networks.

In one preferred embodiment, the SIN vector comprises a fusion nucleic acid comprising a promoter of interest, for example the ε promoter, and a reporter protein, such as GFP. Candidate agents are introduced into or combined with the transformed cells and examined for effects on reporter gene expression, as described in WO 99/58663, hereby expressly incorporated by reference. If the promoter is inducible, promoter is induced with appropriate stimulus or effector. Alternatively, the promoter is induced prior to addition of the candidate bioactive agents, or simultaneously. For example, for the IL-4 inducble ε promoter, addition of cytokines IL-4 or IL-13 to the cells (e.g., IL-4 of not less than 5 units/ml and at a preferred concentration of 200 units/ml) can induce transcription of the ε promoter. Screening of candidate agents affecting inducible expression of the reporter will allow identifying cellular targets involved in signal transduction by the cytokine leading to promoter regulation. To provide a more stringent selection for promoter regulators, the fusion nucleic may comprise a promoter, a reporter gene, a separation sequence, and a selection gene. The reporter gene, such as GFP, allows identification of cells expressing the reporter while the selection gene allows an additional basis for selecting cells. For example, if the selection gene is a thymidine kinase (TK), the cells can be selected based on killing by gangcyclovir since TK activity is needed for gangcyclovir toxicity. Alternatively, the selection gene may encode the HBEGF and the killing initiated by adding the diptheria toxin. Thus, candidate agents that repress promoter activity are readily identified by selecting for cells lacking GFP expression and displaying resistance to cell death. The presence of a separation sequence, such as 2A, permits expression of both reporter and selection genes from a single transcript, thus providing a sensitive indicator of promoter activity.

In another preferred embodiment for studying the regulation of promoter activity, the transformed cells comprise a plurality of SIN vectors comprising a promoter and gene of interest. In one aspect, at least one the plurality of SIN vectors comprises a promoter of interest operably linked to a reporter or selection gene. In addition, at least one of the plurality of SIN vectors comprises a different promoter operably linked to a different gene of interest, which encodes a regulator of the promoter of interest. In one aspect, if the gene of interest are candidate nucleic acids and candidate peptides, and the regulator of the promoter of interest is an inducible transcription factor, such as tetracyclin inducible transcription factor (tTA), expression of the transcription factor allows regulated expression of the candidate agents during the screening process.

In another aspect, if the different gene of interest encodes a regulator of the promoter of interest, cells transformed with these SIN vectors provide stable cell lines for screening of candidate agents affecting the activity of the regulator or signaling pathways in which the regulator acts. For example, it is well known that adenomatosis polyposis coli (APC) protein interacts with β-catenin, a regulator of the Tcf/Lef transcription factor. Phosphorylation by glycogen synthase kinase-3 (GKS-3) of the β-catenin complexed with APC results in rapid degradation of the β-catenin via the ubiquitin degradation pathway. Mutations in APC or β-catenin, however, stabilize β-catenin from degradation, leading to its accumulation and subsequent translocation into the nucleus where it serves as a transcriptional co-activator of Tcf/Lef regulated genes. Moreover, the activity of GKS-3 is regulated, in part, by the Wnt signaling pathway.

Thus, a transformed cell containing at least one SIN vector comprising a Tcf/Lef regulated promoter, such as c-myc or cyclin D1 promoter, which is operably linked to a reporter gene (e.g., GFP) provides a stable cell line for identifying candidate agents regulating Wnt/β-catenin signaling pathways. If the transformed cell further comprises at least another SIN vector comprising a fusion nucleic acid expressing β-catenin or degradation resistant β-catenin variants capable of acting as activators of Tcf/Lef, expressing the β-catenin, either by a constitutive or inducible promoter, results in activation of the promoter of interest, thus providing a more specific cell line for identifying candidate agents affecting β-catenin activity and Tcf/Lef promoter regulation. Candidate agents are combined or introduced into these transformed cells and examined for reduction or loss of expression of the reporter gene to identify candidate bioactive agents capable of disrupting Wnt signaling pathway or β-catenin/Tcf mediated transcriptional activation. Candidate agents with the desired effects are then used to identify the cellular targets affected by the candidate agent. In a further preferred embodiment, the SIN vector expressing the regulator of the promoter of interest may further comprise a separation sequence and second gene of interest encoding a different reporter gene, which allows monitoring the expression of the regulator. Alternatively, the second gene of interest may encode the Tcf/Lef transcription factor to increase β-catenin/Tcf mediate transcriptional activation of the promoter of interest.

In another preferred embodiment, the retroviral vectors and cellular libraries of the present invention are useful in identifying candidate agents affecting proteases involved in pathogenesis. As is well known in the art, viral pathogenesis and cellular physiology is regulated by the activity of various proteases. For example, HIV protease acts on the gag-pol precursor to generate the mature polymerase required for virus replication. This viral protease is a prime target for protease inhibitor based anti-HIV therapies. Other viral proteases are involved in processing of viral polyproteins, which are necessary to produce mature, infectious viral particles. In regards to cellular regulation, caspases comprise a family of proteases involved in activating cell death pathways. Lysozomal proteases, such as the cathepsin family are involved in processing of proteins in the lysozomes and are believed to play a role in metastasis of tumor cells. Extracellular proteases, including metalloproteases act on extracellular matrix to regulate cell-cell interactions. Increased activity of these metalloproteinases are thought to reduce contact inhibition of cells and thus promote growth of tumor cells, including metastasis to other tissues and organs. Tissue inhibitors of extracellular matrix metalloproteases are frequently deleted in certain cancers, such as breast cancer, suggesting that they act to create metastatic potential. Consequently, numerous proteases and biochemical pathways that regulate protease activity serve as important targets for therapeutic agents.

Accordingly, in one embodiment, the SIN vectors of the present invention comprises a fusion nucleic acid comprising a separation sequence recognized by a protease, such as the HIV protease or caspase. The first gene of interest and the second gene of interest encode distinguishable reporter molecules. Thus, in one preferred embodiment, the first gene of interest may comprises a cyan GFP, which is linked via a specific protease recognition site to a second gene of interest, a blue GFP capable of fluorescence resonance energy transfer (FRET). Candidate agents are introduced into cells expressing these protease substrates and the cells screened for agents that inhibit protease acitivity. Candidate agents acting as inhibitors or affecting the regulation of events leading to protease activation will prevent separation of the GFP molecules, thus resulting in increases in the FRET signal.

As an alternative to the FRET based assay, the first reporter gene may be targeted to a cellular location distinguishable from the cellular localization of the second reporter gene. In the absence of a separation reaction, the fusion protein comprising the first reporter protein, protease recognition site, and second reporter protein is directed predominantly to the cellular location of the first reporter protein. For example, the first reporter protein could be targeted to the plasma membrane while the second reporter protein has nuclear localization sequences. In the absence of protease activity, the fusion protein is predominantly localized to the plasma membrane. In the presence of protease, the two reporters are separated, thus allowing the second reporter to properly localize to the nucleus. The redistribution of the reporter protein resulting from protease action allows assessment of protease activity. If the second reporter protein produces a dominant effect on the cell when properly localized to a subcellular compartment, the presence of a dominant effect on a cell provides a useful indicator of protease activity.

In another embodiment for protease substrates, the SIN vectors may comprise a first gene of interest comprising a DNA binding domain while the second gene of interest is a transcriptional activation domain. The sequence linking the DNA binding domain and the transcription activator domain comprises the protease recognition site. In the absence of protease activity, the fusion nucleic acid produces a fusion protein capable of activating transcription of a independent reporter or selection gene construct whose expression is regulated by the fusion protein. The reporter construct is stably integrated in the cell or is introduced into the cell by transfection or viral delivery, for example using the SIN vectors of the present invention. Consequently, the transformed cell may comprise a plurality of SIN vector of which at least one SIN vector expresses the protease substrate and at least one SIN vector provides the reporter construct. Upon expression of the protease under study, separation of the DNA binding domain and transcriptional activation domain occurs, thereby reducing or eliminating transcription of the reporter or selection gene. Candidate agents are then screened for protease inhibiting activity by monitoring increased transcription of the reporter or selection gene. This assay allows high throughput screens to identify protease inhibitors, for example inhibitors of HIV proteases, including variant proteases resistant to protease inhibitor based anti HIV therapy.

In a further preferred embodiment, since many proteases are present extracellularly, the fusion nucleic acids of the present invention may comprise a secretory sequence operably linked to an upstream first gene of interest, preferably encoding a first reporter protein, while a transmembrane anchoring domain sequence is inserted or fused to a downstream second gene of interest, which encodes a second reporter protein. The separation sequence is a peptide region recognized by an extracellular protease, such as a metalloprotease. Upon expression of the fusion nucleic acid in a cell, a fused polypeptide comprising the first protein of interest, protease recognition site, and the second protein of interest is displayed on the cell surface, anchored to the cell membrane via the transmembrane domain. Exposure of the cells to extracellular protease, for example by contact with co-cultured cells expressing the extracellular protease, results in release of the first reporter protein, which is conveniently detected in the cellular medium. Alternatively, the transmembrane domain could be omitted, which releases the protease substrate into the extracellular medium where it can be acted on by proteases. Candidate agents are added to the cells to screen for inhibitors of the extracellular protease. Since metalloproteases and other extracellular proteases are believed to affect the metastatic potential of tumor cells, these types of screen allow for identifying potential anti-metastatic agents.

The protease may be introduced into these transformed cells (or other appropriate cells if the protease is provided by different cells than those expressing the substrate) via an exogenous fusion nucleic acid, for example by retroviral delivery, or transfecting with a nucleic acid construct or incubating with an pathogenic agent expressing the protease. In one aspect, the protease may be provided by a SIN vector. Introducing all components of the assay is also possible by using a fusion nucleic acid comprising a second separating sequence and an additional gene of interest comprising the protease. Thus, this retroviral vector contains the complete protease, protease recognition site, and the appropriate reporter molecules to permit detection of candidate agents acting on the protease. Alternatively, when the protease is an inducible cellular protease, appropriate inducing signals (for example, an apototic signal to induce caspases) are provided to activate the cellular protease.

Since constitutive expression of the protease is potentially cytotoxic, fusion nucleic acids expressing the protease may comprise an inducible promoter while the transformed cell line provides the cognate inducible transcription factor. Thus, in one aspect, the cell used in the assay is transformed with a plurality of SIN vectors wherein at least one SIN vector expresses the inducible transcription factor, at least one SIN vector expresses the protease (i.e. HIV), and at least one SIN vector expresses the substrate for the protease. Candidate agents are combined with or introduced into these cells, and the cells induced to synthesize the protease. These cells are then screened for agents capable of inhibiting protease activity by the assays described above.

In another preferred embodiment, the present invention is useful for identifying candidate agents directed against IRES mediated gene expression. In one aspect, the SIN vectors used to generate transformed cells may comprise a fusion nucleic acid in which the separation site is an IRES element derived from a pathogenic virus, such as hepatitis C virus (HCV) IRES, or a cellular IRES element responsible for expression of gene products involved in cellular disease states. The transformed cell comprises a SIN vector comprising a first gene of interest encoding a first reporter/selection gene, an IRES element, and a second gene of interest encoding a second reporter/selection gene. In this embodiment, the IRES element preferably regulates expression of the downstream gene of interest. Cells transformed with these SIN vectors are selectable based on expression of both first and second genes of interest. Candidate agents are introduced into these cell lines, for example by retroviral delivery, and screened for their ability to inhibit IRES dependent expression of the second reporter/selection gene. The first reporter/selection gene serves as a useful monitor for expression of the fusion nucleic acid and for distinguishing inhibitory effects of candidate agents on transcription as compared to translation. Candidate agents and their cellular targets are identified, which may lead to therapeutic agents effective against diseases dependent on IRES mediated gene expression.

Similarly, another aspect of the present invention comprises SIN vectors in which the separation site is a Type 2A sequence from a pathogenic virus or a Type 2A sequence mediating expression of a gene product responsible for a cellular disease state. In assays similar to those described above, the fusion nucleic acids comprise a first reporter/selection gene, a Type 2A separation sequence, and a second reporter/selection gene. Thus, the fusion nucleic acid expresses separate reporter/selection proteins encoded by the first and second genes of interest. These expressing cells are treated with candidate agents to identify inhibitors of the 2A separating activity as indicated by the production of unseparated proteins encoded by the first and second genes of interest. For example, the assays may incorporate use of GFP based FRET, whereby inhibition of 2A separation activity results in increased FRET signal arising from retention of linkage between GFP reporter molecules. If the assay uses cellular localization of the reporter proteins as the basis to detect separate reporter/selection proteins, inhibition of 2A separating activity will result in altered cellular localization of the reporter/selection genes. Alternatively, when the first and second reporter genes encode a DNA binding domain and a transcriptional activation domain, respectively, inhibiting the Type 2A separation activity results in expression of a functional transcriptional regulator capable of increasing expression of an independent reporter construct.

In another preferred embodiment, cells transformed with SIN vectors find use in screening for cells with altered exocytosis phenotypes. By “alteration” or “modulation” in relation to exocytosis is meant a decrease or increase in amount or frequency of exocytosis in one cell compared to another cell or in the same cell under different conditions. Often mediated by specialized cells, exocytosis is vital for a variety of cellular processes, including neurotramitter release by neurons, hormone release by adrenal chromaffin cells (adrenaline) and pancreatic β-cells (insulin), and histamine release by mast cells.

Disorders involving exocytosis are numerous. For example, inflammatory immune response mediated by mast cells leads to a variety of disorders, including asthma and allergies. Therapy for allergy remains limited to blocking mediators released by mast cells (i.e., anti-histamines) and non-specific anti-inflammatory agents, such as steroids and mast cell stabilizers. These treatments are only marginally effective in alleviating the symptoms of allergy. To identify cellular targets for drug design or candidate effectors of exocytosis, SIN vectors comprising libraries of candidate agents may be introduced into appropriate cells, for example mast cells, and selected for modulation of exocytosis by assaying for changes in cellular exocytosis properties. These cells are stimulated with appropriate inducer if exocytosis is triggered by an inducing signal.

Assays for changes in exocytosis may comprise sorting cells in a fluorescence cell sorter (FACS) by measuring alterations of various exocytosis indicators, such as light scattering, fluorescent dye uptake, fluorescent dye release, granule release, and quantity of granule specific proteins (as provided in U.S. Ser. No. 09/293,670, hereby expressly incorporated by reference). Use of combinations of indicators reduces background and increases specificity of the sorting assay.

The exocytosis assay based on changes in the cell's light scattering properties, including use of forward and side scatter properties of the cells, are indicative of the size, shape, and granule content of the cell. Multiparameter FACS selection based on light scattering properties of cells are well known in the art, (see Perretti, M. et al. (1990) J. Pharmacol. Methods 23: 187-94; Hide, I. et al. (1993) J. Cell Biol. 123: 585-93).

Assays based on uptake of fluorescent dyes reflect the coupling of exocytosis and endocytosis in which endocytosis levels indirectly reflect exocytosis levels since the cell attempts to maintain cell volume and membrane integrity as the amount of cell membrane rapidly changes when secretory vesicles fuse with the cell membrane. Preferred fluorescent dyes include styryl dyes, such as FM143, FM4-64, FM14-68, FM2-10, FM4-84, FM1-84, FM14-27, FM14-29, FM3-25, FM3-14, FM5-55, RH414, FM6-55, FM10-75, FM1-81, FM9-49, FM4-95, FM4-59, FM9-40, and combinations thereof. Styryl dyes such as FM1-43 are only weakly fluorescent in water but very fluorescent when associated with a membrane, such that dye uptake by endocytosis is readily discernable (Betz, et al. (1996) Current Opinion in Neurobiology, 6:365-371; Molecular Probes, Inc., Eugene, Oreg., “Handbook of Fluorescent Probes and Research Chemicals”, 6th Edition, 1996, particularly, Chapter 17, and more particularly, Section 2 of Chapter 17, (including referenced related chapter), hereby incorporated herein by reference). Useful solution dye concentration is about 25 to 1000-5000 nM, with from about 50 to about 1000 nM being preferred, and from about 50 to 250 being particularly preferred.

Exocytosis assays based on fluorescent dye release rely on release of dye that is taken up passively by the cell or dye that is actively endocytosed by the cell. Release of dyes initially taken up by a cell results in decreased cellular fluorescence and presence of the dye in the cellular medium, thus providing two basis for measuring dye release. For example, styryl dyes taken up into cells by endocytosis is released into the cellular media by exocytosis, resulting in decreased cellular fluorescence and presence of the dye in the medium. Another dye release assay uses low pH dyes, such as acridine orange, LYSOTRACKER™ red, LYSOTRACKER™ green, and LYSOTRACKER™ blue (Molecular Probes, supra), which stain exocytic granules when dye is internalized by the cell.

Preferential staining of exocytic granules when the vesicles fuse with the cell membrane provides an additional assay for measuring exocytosis. Annexin V, which binds to phospholipid (phospahtidyl serine) in a divalent ion dependent manner, specifically binds to exocytic granules present on the cell surface but fails to bind internally localized exocytic granules. This property of Annexin provides a basis for determining exocytosis by the level of Annexin bound to cells. Cells show an increase in Annexin binding in proportion to the time and intensity of the exocytic response. Annexin is detectable directly by use of fluorescently labeled Annexin derivatives (e.g., FITC, TRITC, AMCA, APC, or Cy-5 fluorescent labels), or indirectly by use of Annexin modified with a primary label (e.g., biotin), which is detected using a labeled secondary agent that binds to the primary label (e.g., fluorescently labeled avidin).

Alternatively, in a preferred embodiment the exocytosis indicators are engineered into the cells. For example, recombinant proteins comprising fusion proteins of a granule specific, or a secreted protein, and a reporter molecule are expressed in a cell by transforming the cells with a fusion nucleic acid encoding a fusion protein comprising a granule specific or secreted protein and a reporter protein. This is generally done as is known in the art, and will depend on the cell type. Generally, for mammalian cells, retroviral vectors, including the SIN vectors described herein, are preferred for delivery of the fusion nucleic acid. Preferred reporter molecules include, but are not limited to, Aequoria Victoria GFP, Renilla mulleris GFP, Renilla reniformis GFP, Renilla ptilosarcus, GFP, BFP, YFP, and enzymes including luciferases (Renilla, firefly etc.) and p-galactosidases. Presence of the granule protein-reporter fusion construct on the cell surface or presence of secreted protein-reporter fusion construct in the medium indicates the level of exocytosis in the cells. Thus, in one preferred embodiment cells are transformed with SIN vectors expressing a fusion protein comprising granule specific (i.e., secretory vesicle) proteins, such as VAMP (synaptobrevin) or synaptotagmin, fused to a GFP reporter molecule. The cells are monitored for localization of the fusion protein to the cell membrane. Candidate agents, for example candidate nucleic acids and candidate proteins, introduced into these transformed cells are tested for their ability to affect distribution of the fusion protein. Since the definition of granule specific proteins encompasses mediators released during exocytosis, including, but not limited to, serotonin, histamine, heparin, hormones, etc., these granule proteins may be identified using specific antibodies.

In another preferred embodiment, the present inventions are useful in screening for agents affecting cell cycle regulation. It is known that the cell cycle is regulated by a complicated network of regulatory pathways involving molecules such as cell surface receptors, cyclins, cyclin dependent kinases, kinase inhibitors, phosphatases, tumor suppressors, transcription factors, and components of the ubiquitin mediated protein degradation pathway (e.g., ubiquitin conjugating enzyme, ubiquitin ligase, preoteasome complex, etc.). Dysregulation of the cell cycle leads to a variety of disease states, for example tumor formation and improper immune system response. To identify candidate agents affecting cell cycle regulation, cells with senescent or proliferative properties are transformed with SIN vectors expressing a library of candidate agents, for example random peptides. In one aspect, the SIN vector may further comprise a separation sequence and a second gene of interest encoding a reporter gene for detecting expression of the random peptide. Presence of the separation sequence limits any interference of the reporter protein on the function of the candidate agent. The promoter is constitutive or inducible, but an inducible promoter allows examining the cellular phenotype in the absence of expressed peptide or in the presence of expressed peptide, which is important for distinguishing between altered cellular phenotypes caused by somatic mutations and candidate agents. Cells are then examined for effects on the cell cycle, for example by analysis of cell viability, cellular DNA content, cell proliferation assays, etc. (see US 2001/0003042, hereby incorporated by reference). These cellular parameters are readily measured by methods well known in the art (e.g., FACS analysis). Furthermore, the cells may be transformed with a plurality of SIN vectors where, in addition to the fusion nucleic acid expressing the candidate nucleic acid, at least one of the SIN vectors also comprises a fusion nucleic acid encoding a reporter protein that communicates the cell cycle status of the cell, for example a GFP fused to a chromatin associated protein (see Belmont, A. D. (2001) Trends Cell Biol. 11: 250-57; Kimura, H. et al. (2001) J. Cell. Biol. 153:1341-53) or a cyclin destruction box. These methods outlined above permit identification of candidate agents having specific effects on the cell cycle and allow isolation of the cognate cellular target molecules involved in cell cycle regulation.

In another embodiment, the SIN vectors are used to express cell cycle regulators or mutant variants of cell cycle regulators, which produce an aberrant cell cycle phenotype in the transformed cells. Thus, in one aspect, the SIN vectors may comprise fusion nucleic acids overexpressing a cell cycle regulator, such as cyclin (Cln). Moreover, the SIN vectors of the present invention is used to express combinations of cells cycle regulators, such as Cln and cyclin dependent kinase (Cdk), to dysregulate Cdk pathways and generate aberrant cell cycles. These transformed cells serve as screening systems to identify candidate agents affecting cellular targets involved in regulating cell cycle pathways.

In another preferred embodiment, the transformed cells are useful in signal transduction applications, especially in disease states involving dysregulation of signal transduction pathways. For example, it is well known that mutations or inappropriate expression of genes such as Her/Neu, Erb, Abl, Src, Ras, Raf, Rb, and p53, among others, induce abnormal cell growth phenotype arising from disrupted signal transduction. The signal transduction events affected in these cells may arise from inappropriate cell surface receptor activation, dysfunctional kinase activity, unregulated protein-protein interactions, mistranscription of genes, etc. In one aspect, the present invention is used to treat the affected signal transduction pathway by identifying candidate agents that reverse the effects of signal transduction misregulation. A library of SIN vectors expressing candidate nucleic acids and peptides are used to transform cells having defects in signal transduction, such as tumor cells expressing constitutively active Ras or Rb proteins. Cells with altered phenotype, for example loss of contact inhibition or growth in soft agar, are identified and the bioactive agent identified.

In another aspect, cells are transformed with SIN vectors comprising fusion nucleic acids expressing signal transduction proteins, or mutant variants thereof, that when expressed in a cell induce a specific cellular phenotype. For example, expression of oncogenes (e.g., Src, Ras, Raf) in particular cell types are known to induce a tumorigenic phenotype. Candidate agents are introduced into these cells, and cells in which tumorigenic phenotype is reversed or increased is identified. Alternatively, cells are transformed with a plurality of SIN vectors where at least two of the SIN vectors express proteins which act together or synergistically to produce a tumorigenic phenotype. For example, it is well known that Ras and Raf oncogenes interact to transform cells by activating the ras signaling pathway. By expressing these combination of proteins, non-tumorigenic cells can be induced to display tumorigenic phenotype. In addition to use of plurality of SIN vectors, these proteins may also be expressed using SIN vectors comprising a first gene of interest, separation sequence, and second gene of interest. Once these transformed cells are available, screens may be conducted for candidate agents and cellular targets that specifically reverse, enhance, or modulate the dominant phenotype caused by the expressed proteins.

In yet another preferred embodiment, the present invention is useful in screening for modulators of cell death pathways. A variety of diseases states are associated with inhibition or activation of cell death pathways. Inhibiting cell death pathways may result in cell proliferation and tumorigenesis while inflammatory responses can activate cell death pathways leading to cell apoptosis.

In one aspect, candidate agents are screened for anti-death gene activity. Cell death is initiated by activating cell death pathway, for example by using a cell death ligand (e.g., Fas ligand). In another aspect, cells are transformed with SIN vectors comprising fusion nucleic acids expressing death inducing genes. For example, the cells are transformed with a SIN vector expressing caspases or ICE related proteases. Use of an inducible promoter limits the detrimental effect of constitutive expression. Candidates agents are introduced into these cells and then cell death induced by activating expression of the cell death gene. Transformed cells surviving the induction of the death gene is isolated and the candidate agents providing anti-death protection identified. Cell death assays are well known in the art (e.g., annexin-phycoerythrin staining; see also US 2001/0003042).

In another embodiment, the transformed cells express multiple death promoting genes to activate multiple cell death pathways. In addition, the transformed cells may express multiple cell death related proteins when interaction of multiple proteins is required to induce a particular cell death pathway. Thus, in one aspect, a transformed cell may comprise a plurality of SIN vectors expressing at least two different caspases to activate independent cell death pathways. In another example, the transformed cells may express caspase 9 and Apaf-1, which are known to interact and form the apoptosome complex that leads to induction of cell death. As indicated above, expression of the cell death proteins are preferably under the control of an inducible promoter. Candidate agents are combined or introduced into these cells and cell death induced by expressing the cell death genes to screen for agents and cellular targets acting on cell death pathways.

In another preferred embodiment, the present invention is used in various drug applications. Drug toxicity is a significant clinical problem and can limit the effectiveness of particular drugs. For example, many cancer therapies rely on generalized DNA damage by agents, such as cisplatin, adriamycin or bleomycin, etc. while some anti-cancer compounds, including vinblastin, vinchristine and Taxol, act on the cell microtubule machinery. Selectivity of these drugs is based on differential growth of cancerous cells versus normal cells, but the general lack of specificity of these compounds results in toxicity to normal cells as well as to cancer cells. Selectivity may be increased by increasing the sensitivity of cancer cells to anti-cancer compounds or by protecting normal cells from the toxic effects of the drug. In one aspect, non-cancerous cells are transformed with a library of SIN vectors expressing the candidate agents and treated with the drug to identify candidate agents that protect the cells from the toxic effects of the drug. In another aspect, cancer cells are transformed with SIN vectors expressing candidate nucleic acids or peptides and treated with the drug to identify agents that sensitizes the cells to the drug. The assay may involve detecting apoptotic markers, DNA fragmentation, microtubule dynamics, or cell viability staining.

In other drug related applications, it is well known that expression of ATP cassetted transporters confers multi-drug resistance upon cells. This effect is readily seen in populations of cancer cells treated with anti-cancer agents in which drug toxicity provides a selection pressure for growth of cells resistant to the drug, thereby reducing the drug's efficacy in treating the cancer. Since drug resistance may arise from multiple factors, use of cultured cancer cells may limit the likelihood of identifying candidate agents acting on specific cellular targets involved in development of drug resistance. This problem is obviated by using cells transformed with SIN vectors expressing genes, such as MDRI, MRP, MCRP, MXR or combinations thereof, that confer drug resistance upon a cell. A plurality of SIN vectors, or a SIN vector comprising a fusion nucleic acid comprising a gene of interest, separation sequence, and a second gene of interest, are used to express various combinations of multi-drug resistance proteins in cells. When an individual multi-drug resistance gene is expressed in a cell, candidate agents capable of optimally inhibiting each of the separate transporters may be identified. These agents then may be combined to provide a combination therapy to inhibit a group of transporters expressed in drug resistant cancer cells. Alternatively, when combinations of multi-drug resistance genes are expressed in a cell, candidate agents capable of inhibiting the group of multi-drug resistance genes may be identified. Comparison of all identified candidate agents should allow design of additional candidate agents effective against the expressed multi-drug resistance genes.

In another preferred embodiment, the present invention is useful in inflammation and immunology applications. The inflammatory response is mediated, in part, by cyclooxygenases (COX1 and COX2), nitric oxide synthase (NOS), and heme oxygenase. Activity of these enzymes are implicated in cell death, tumor progression, and immune response. For example, increase in the inducible form of NOS (iNOS) in immune cells following tissue injury, for example brain ischemia, may lead to cell death of cells surrounding the injury sight. In part, the mechanism for toxicity of increased NO production is believed to be activation of cell death pathways. The endothelial form of NOS (eNOS) found in the cardiovascular system produces NO, which functions as a vasodilator, and provides the basis for drugs effective for treating angina and erectile dysfunction. The neuroal form of NOS (nNOS) in the peripheral and central nervous system produces NO, which functions as a neuromodulator. Consequently, finding specific inhibitors of the various forms of NOS have wide ranging applications in the clinical setting.

In the present invention, cells may be transformed with SIN vectors expressing various forms of NOS. The cell may contain a single form of NOS or combinations of the NOS forms. If constitutive expression is injurious to the cells, inducible promoters (i.e. tetp) are used to regulate NOS expression. As described above, an inducible transcription factor (i.e. tTA) may be provided in the transformed cell by at least one of the plurality of SIN vectors. Candidate agents are combined with or introduced into these transformed cells and the cells examined for synthesis of NO by methods well known in the art (e.g., FACS; see Nakatsubo, N. et al. (1998) FEBS Letters 427: 263-66; Kojima, H. et al. (1998) Chem. Pharm. Bull. 46: 373-75). Cells with low NOS activity are isolated and the candidate agent identified. This method may be applied generally to cyclooxygenases and heme-oxygenase or other enzymes involved in mediating the inflammatory response.

In yet another preferred embodiment, the present invention is useful in identifying modulators of the immune response. For example, activation of B-cells initiates various facets of humoral immunity, including immunoglobulin synthesis and antigen presentation by B-cells. Activation is mediated by engagement of the B-cell receptor (BCR), for example by binding of anti-lgM F(ab′) fragments, which induces several signal transduction pathways leading to various responses by the B-cell, including apoptosis, expression of cell surface marker CD69, and modulation of IgH promoter activity. In one aspect, the SIN vectors of the present invention are useful for introducing candidate agents, such as libraries of cDNAs, candidate nucleic acids, and candidate peptides into appropriate B-cell lines, such as Ramos Human B-cell lines, M12.4, MC116, DND39, etc., to identify various effectors of the signaling pathways activated by B-cell receptor engagement. The effectors may be the candidate agents themselves or the cellular targets of the candidate agents, and the assay may comprise determining the level of CD69 cell surface marker (e.g., by fluorescently labeled anti-CD69 antibody and FACS selection of cells expressing high levels of CD69) or inhibition of apoptotic pathway following receptor activation.

In another aspect, the present invention is useful as indicators of B-cell receptor mediated signal transduction. In one preferred embodiment, the SIN vector comprises an IgH promoter operably linked to a reporter gene (e.g., GFP), or to a first gene of interest comprising a reporter gene, a separation sequence, and a second gene of interest comprising a second reporter or selection gene. For example, the genes of interest may comprise a combination such as GFP and HBEGF, which provides selection based on GFP expression and diptheria toxin mediated killing (see WO 0134806, hereby incorporated by reference). This and other configurations provide sensitive monitoring of BCR activation by the detecting IgH promoter activity. Candidate agents are introduced into these cells to identify agents that activate or suppress BCR mediated signal transduction, as reflected by changes in IgH promoter activity. Expression of the candidate agents may be under the control of an inducible promoter, such as tetP, thus limiting any detrimental effect on the cell by constitutive expression of candidate agents. Inducible expression of candidate agents also provides a basis for distinguishing between altered cellular phenotypes caused by somatic mutations and candidate agents. Generally, cells used in this type of screen will also a comprise fusion nucleic acid expressing the tetracyclin regulatable transactivators (see for example, Goose, N. M. et al. (1995) Science 268: 1766-69).

Thus, in a preferred embodiment, a transformed cell used to identify candidate agents affecting BCR mediated signal transduction may comprise a plurality of SIN vectors where at least one SIN vector comprises a fusion nucleic expressing a tetracycline inducible transcription factor (tTA) and at least one SIN vector comprises a fusion nucleic acid comprising the tetP promoter operably linked to fusion nucleic acids expressing candidate agents. Depending on the screening method used, the cells may optionally have at least one SIN vector comprising an IgH promoter operably linked to a reporter gene. These cells, initially grown in the presence of tetracycline analog (Doxycycline) to repress candidate gene expression, are induced by removal of the analog to initiate expression of candidate agents. Treatment with anti-lgM F(ab′)2 fragments activates BRC pathways, and the cells are screened based on the assays described above. Upon identification of bioactive candidate agents, the cellular targets of the candidate agent can be isolated.

In another embodiment, the present invention is used in anti-viral applications. For example, HIV is the etiological cause of acquired immune deficiency syndrome (AIDS), which exacts a enormous social and financial costs on society. Therapeutic targets for inhibiting replication of the virus are generally directly towards inhibiting reverse transcriptase or viral proteases required for viral replication. The promiscuity of reverse transcriptase, however, results in rapid accumulation of mutations that renders the reverse transcriptase or protease resistant to the drugs directed towards these enzymes. Continual development of drugs targeting the resistant enzymes or development of new targets are needed for HIV directed therapies.

In one preferred embodiment, the SIN vectors comprising fusion nucleic acids expressing candidate agents are used to transform cells susceptible to infection by HIV virus. These transformed cells are infected with HIV virus, including resistant forms of the virus, and examined to identify cells resistant to virus replication. Cells which are not normally susceptible to infection are induced to being susceptible by transforming the cells with the HIV virus receptor, CD4, which is readily introduced into the cells via SIN vectors expressing a gene of interest encoding the CD4 molecule. Cells resistant to viral replication are identified based on absence of cytopathological effects on the infected cells (e.g., apoptosis) and/or presence of viral proteins in the cell (e.g., as determined by antibodies to presence of viral proteins).

It is understood by the skilled artisan that the steps for constructing the SIN vectors, fusion nucleic acids, retroviral libraries, and cellular libraries can be varied according to the options provided herein. Those skilled in the art may modify according to the skill in the art

The following examples serve to more fully describe the manner of using the above-described invention for carrying out various aspects of the invention. It is understood that these embodiments in no way serve to limit the scope of this invention. All references cited herein are incorporated by reference in their entirety.

### EXAMPLES

### Example 1

### Construction of a Promoter-Reporter Cell Line

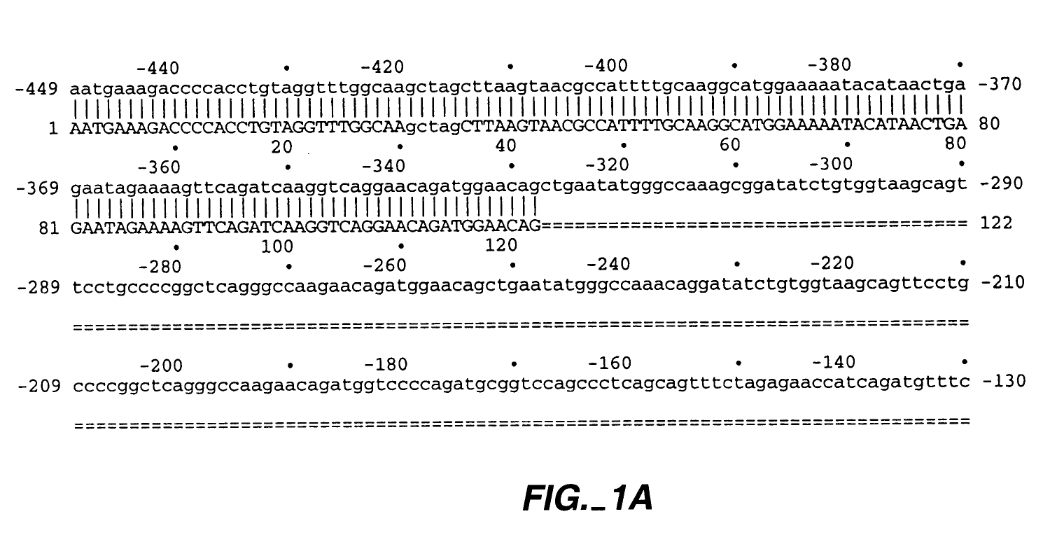

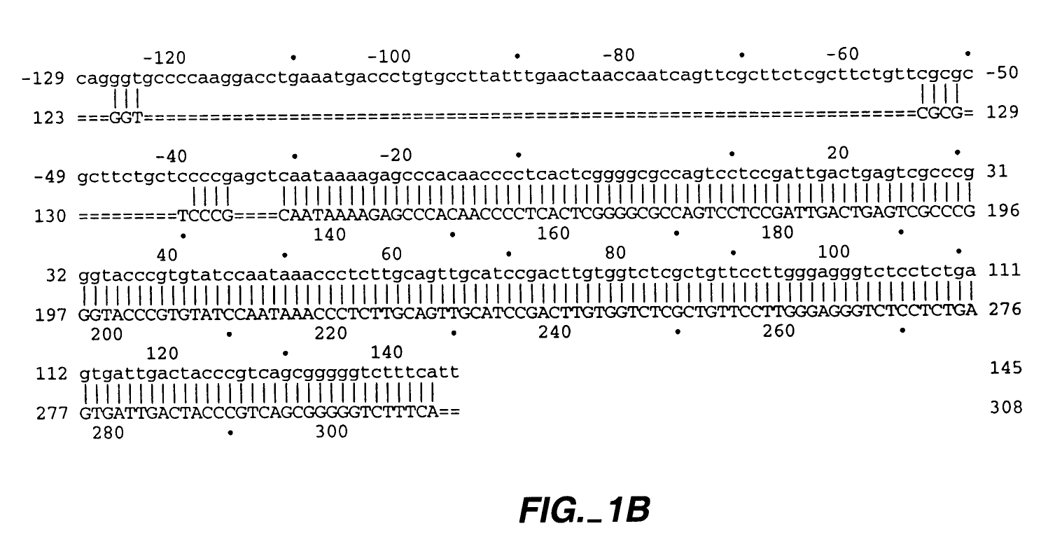

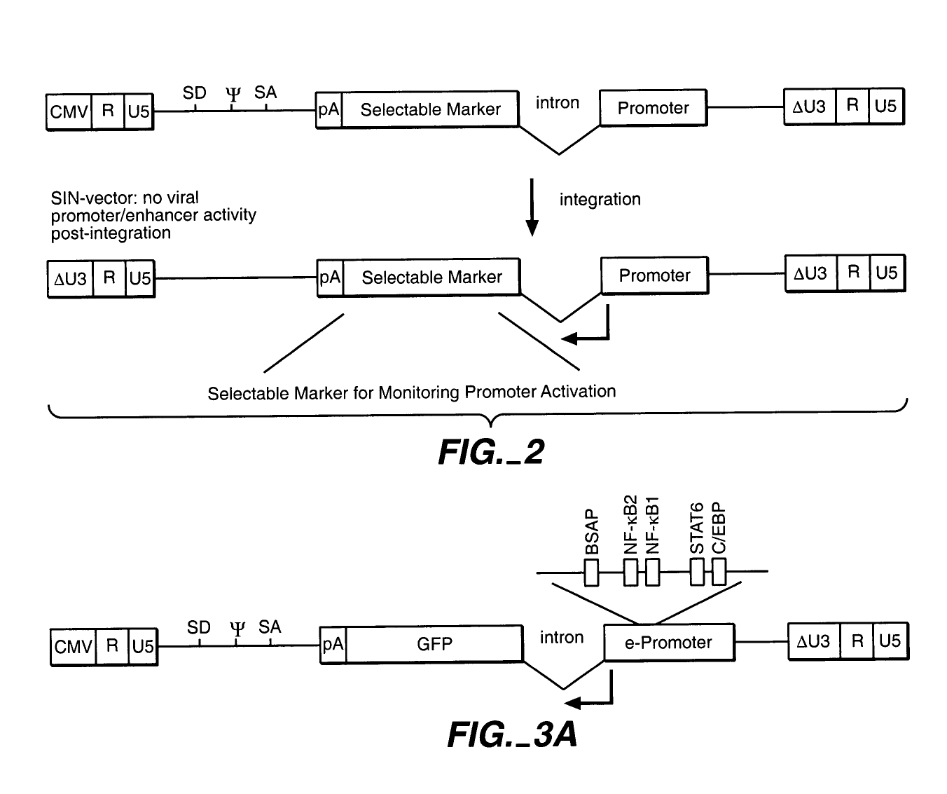

Reporter construct for examining IgM ε promoter activity is shown in FIG. 3. The reporter construct is based on CRU5 (Naviaux et al. “The pCL Vector System: Rapid Production of Helper Free, High Titre, Recombinant Retroviruses,” J. Virol. 70: 5701-05 (1996)) vector, which uses a CMV promoter located near the 5′ end of the viral genome to transcribe RNAs for packaging into virus particles. The 3′ end of the construct contains a SIN deletion in the U3 region (AU3; as provided in FIG. 1) of the 3′ LTR (i.e., ΔU3-R-U5). An IL-4 responsive 600 bp fragment of the ε promoter is linked to a GFP reporter gene via a β-globin intron, and a poly adenylation site, pA, is present near the 3′ end of the GFP gene to allow efficient protein expression. Extended packaging signal ψ⁺ is present for packaging of transcribed, non-spliced RNA molecules. Viral sequences and construction of the vectors are further provided in WO 0134806, hereby incorporated by reference. The described construct is transfected into 293 based Phoenix packaging cell lines to generate retroviral particles (Swift, et al., In Current Protocols in Immunology (J. E. Coligan, A. M. Kruisbeek, D. H. Marguiles, E. M. Shevach, and W. Strober, Eds.), Vol. 1017 C, ppl-17, Wiley, New York).

Filtered virus was used to infect Burkitt's Lymphoma cell line CA46, and the cell population analyzed by FACS with or without stimulation with about 30 U/ml of IL-4 for about 2-3 days. Flow cytometric analysis was conducted on a FACS Caliber flow cytometer (BD-Biosciences, Franklin Lakes, N.J.). FACS data was analyzed using WinList (Verity Software House, Topsham, Me.) analysis program. Uninfected cells provided a baseline fluorescence for comparison to infected cells.

Cells with high GFP expression following IL-4 stimulation was selected by FACS, grown for several days, and then reselected for low GFP fluorescence in the absence of IL-4. Following several rounds of screening in the presence and absence of IL-4, the D5 cell line was selected. This cell line does not express GFP in the absence of IL-4, but expresses high levels of GFP in the presence of IL4 stimulation, suggesting that the promoter reporter cell line is a highly sensitive indicator of IL-4 mediated activation of the ε promoter (see FIG. 3B).

### Example 2

### Screens for Candidate Agents Affecting BCR Mediated Activation of IgH Promoter

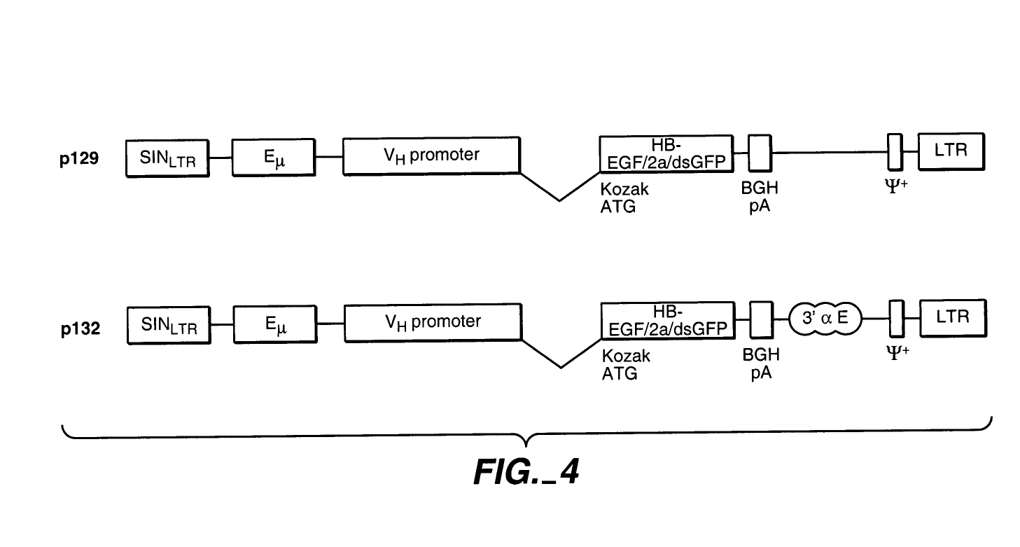

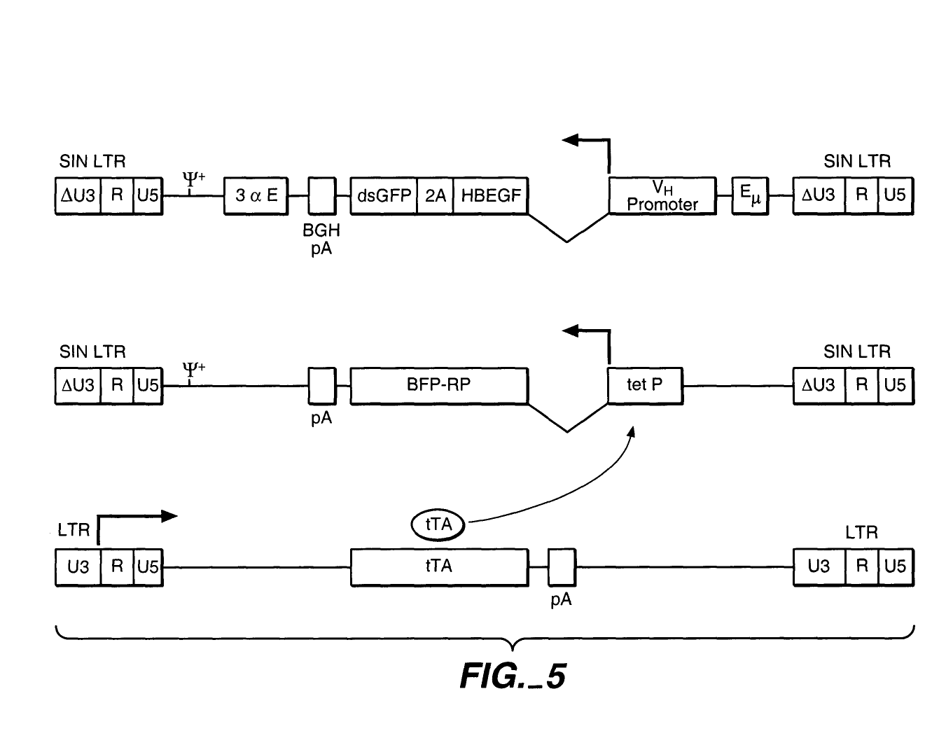

The SIN vector used in the screen is the p132 construct shown in FIG. 4. Promoter elements comprise an IgH V_{H} promoter, the intronic enhancer Eμ (see Lin, M. M. et al (1998) Int. Immunol. 10: 1121-9), and a 3′ enhancer element, 3′αE (Lin, et al., supra). A β-globin intron ((see Lorens et al. (2000) Virology 272: 7-15) and bovine growth hormone poly adenylation sequences are used to efficiently express the genes of interest, which comprise HBEGF as a first gene of interest, a FMDV 2A separation sequence (Donnelly, M. L. et al. (1997) J. Gen. Virol. 78: 13-21), and destabilized GFP (Clontech, Palo Alto, Calif.). The construct was made in a pCRU5 base vector and transfected into 293 based Phoenix packaging cells to generate viruses, which were collected from the culture medium. Infections were generally carried out by spin infection with 0.45 um filtered virus containing medium.

BJAB-tTA cells, a B-cell line which expresses the tetracyclin regulatable transactivator, was transduced with p132 viral constructs and cells selected by FACS based on low GFP expression in the absence of anti-IgM F(ab)2 antibody stimulation and for high levels of expression in presence of antibody. Optimal activation of IgH promoter occurs at an anti-lgM antibody concentration of about 2 ug/ml. Increase in GFP expression are seen to about 40-48 hrs following antibody treatment. Additional selection based on sensitivity to diptheria toxin is optional since the basal level of IgH promoter activity is sufficiently high in the absence of IL-4 induction. After several rounds of selection, cell lines that display high level of GFP expression upon BCR activation and low GFP expression in absence of receptor stimulation were selected as screening cell lines.

For screening candidate agents, a cDNA or a BFP-RP random peptide fusion library was constructed in pTRA vector (see Lorens et al., supra) and packaged in 293 based Phoenix packaging cells. Viral supernatants were collected and used to infect about 2×10⁸ BJAB tTA cell lines containing the p132 promoter reporter construct. Cells were selected by FACS based on low GFP expression, grown for about 4-5 days, and reselected. The low GFP expressing cells were then treated with tetracyclin analog, doxcyclin, at about 100 ng/ml to repress expression of candidate agents. Following additional growth for about 5-6 days, FACS was used to select single cells exhibiting high GFP expression. Retesting the identified cells for doxycyclin regulatable GFP expression identifies candidate agents that regulate BCR mediated activation of the IgH promoter. Two rounds of stimulation and selection are generally used to identify cells expressing bioactive candidate agents.

We claim:

1. A method of screening cells comprising:
a) providing a plurality of transformed cells, each said cell transformed with a retroviral self-inactivating (SIN) vector comprising a promoter operably linked to a first gene of interest;
b) combining said cells with at least one candidate agent; and
c) screening said cells for an altered phenotype.

2. A method according to claim 1, wherein said SIN vector comprises
a. said promoter
b. said first gene of interest
c. a separation sequence; and
d. a second gene of interest.

3. A method according to claim 2, wherein said separation sequence comprises a protease recognition sequence.

4. A method according to claim 2, wherein said separation sequence comprises an IRES sequence.

5. A method according to claim 2, wherein said separation sequence comprises a Type 2A sequence.

6. A method according to claim 1 or 2, wherein said gene of interest comprises a reporter gene.

7. A method according to claim 6, wherein said reporter gene comprises GFP.

8. A method according to claim 7, wherein said GFP comprises Aequoria victoria GFP.

9. A method according to claim 7, wherein said GFP comprises Renilla reniformis GFP.

10. A method according to claim 7, wherein said GFP comprises Renilla mulleris GFP.

11. A method according to claim 7, wherein said GFP comprises Ptilosarcus gurneyi GFP.

12. A method according to claim 1 or 2, wherein said gene of interest comprises a selection gene.

13. A method according to claim 1 or 2, wherein said gene of interest comprises a nucleic acid encoding a dominant effect protein.

14. A method according to claim 1 or 2 of screening for said candidate agent which regulates activity of said promoter, wherein detecting said altered phenotype comprises detecting presence or absence of expression of said gene of interest.

15. A method according to claim 14, wherein said promoter comprises an inducible promoter and said method further comprises inducing said promoter with an inducer.

16. A method according to claim 15 wherein said promoter comprises an IL-4 inducible ε promoter and said inducer comprises IL-4.

17. A method according to claim 14, wherein said gene of interest comprises a reporter gene.

18. A method according to claim 17, wherein said reporter gene comprises GFP.

19. A method according to claim 17, wherein said reporter gene encodes a death gene that is activated by the introduction of a ligand.

20. A method according to claim 1 or 2, wherein each said cell comprises multiple SIN vectors.

21. A method according to claim 20 wherein said promoters of multiple SIN vectors is the same.

22. A method according to claim 20 wherein said promoters of multiple SIN vectors is different.

23. A method according to claim 20, wherein said gene of interest of multiple SIN vectors is different.

24. A method according to claim 20, wherein at least one of said SIN vectors comprises a gene of interest encoding a regulator of a different promoter of at least one of said SIN vectors.

25. A method according to claim 1 or 2, wherein said candidate agent comprises a small molecule.

26. A method according to claim 1 or 2, wherein said candidate agent comprises cDNA.

27. A method according to claim 1 or 2, wherein said candidate agent comprises cDNA fragment.

28. A method according to claim 1 or 2, wherein said candidate agent comprises genomic DNA fragment.

29. A method according to claim 1 or 2, wherein said candidate agent comprises random peptide.

30. A method according to claim 29, wherein said random peptide is biased.

31. A method according to claim 1 or 2, wherein said combining comprises transducing said plurality of cells with a retroviral vector comprising nucleic acids encoding said candidate agent.

32. A method according to claim 1 or 2 further comprising isolating said cell with said altered phenotype.

33. A method according to claim 32 further comprising identifying the candidate agent producing said altered phenotype.


SEQUENCE LISTING (53 sequences)

SEQ ID NO: 1 - DNA, 594 bp, Moloney murine leukemia virus
aatgaaagaccccacctgtaggtttggcaagctagcttaagtaacgccattttgcaaggcatggaaaaatacataactgagaatagaaaagttcagatcaaggtcaggaacagatggaacagctgaatatgggccaaagcggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 2 - DNA, 308 bp, Artificial sequence
  synthetic
aatgaaagaccccacctgtaggtttggcaagctagcttaagtaacgccattttgcaaggcatggaaaaatacataactgagaatagaaaagttcagatcaaggtcaggaacagatggaacagggtcgcgtcccgcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 3 - protein, 21 aa, Artificial Sequence
  Type 2A consensus sequence
XXXXXXXXXXLXXDXEXNPGP

SEQ ID NO: 4 - protein, 61 aa, Artificial sequence
  coiled-coil presentation structure
MGCAALESEVSALESEVASLESEVAALGRGDMPLAAVKSKLSAVKSKLASVKSKLAACGPP

SEQ ID NO: 5 - protein, 69 aa, Artificial sequence
  minibody presentation structure
MGRNSQATSGFTFSHFYMEWVRGGEYIAASRHKHNKYTTEYSASVKGRYIVSRDTSQSILYLQKKKGPP

SEQ ID NO: 6 - protein, 32 aa, Artificial Sequence
  zinc finger consensus sequence
XXXXXCXXXCXXXXXXXXXXXXHXXXHXXXXX

SEQ ID NO: 7 - protein, 33 aa, Artificial Sequence
  C2H2 zinc finger consensus sequence
FQCEECXXXXXXXXXXXXXXXXXXXXHIRSHTG

SEQ ID NO: 8 - protein, 30 aa, Artificial sequence
  CCHC box consensus sequence
CXXCXXXXXXXXXXXXXXXXXXXXHXXXXC

SEQ ID NO: 9 - protein, 33 aa, Artificial sequence
  CCHC box consensus sequence
VKCFNCXXXXXXXXXXXXXXXXXXXXHTARNCR

SEQ ID NO: 10 - protein, 34 aa, Artificial sequence
  CCHC box consensus sequence
MNPNCARCGXXXXXXXXXXXXXXXXXXXXHKACF

SEQ ID NO: 11 - protein, 7 aa, Simian virus 40
PKKKRKV

SEQ ID NO: 12 - protein, 6 aa, Homo sapiens
ARRRRP

SEQ ID NO: 13 - protein, 10 aa, Mus musculus
EEVQRKRQKL

SEQ ID NO: 14 - protein, 9 aa, Mus musculus
EEKRKRTYE

SEQ ID NO: 15 - protein, 20 aa, Xenopus laevis
AVKRPAATKKAGQAKKKKLD

SEQ ID NO: 16 - protein, 31 aa, Mus musculus
MASPLTRFLSLNLLLLGESILGSGEAKPQAP

SEQ ID NO: 17 - protein, 21 aa, Homo sapiens
MSSFGYRTLTVALFTLICCPG

SEQ ID NO: 18 - protein, 51 aa, Mus musculus
PQRPEDCRPRGSVKGTGLDFACDIYIWAPLAGICVALLLSLIITLICYHSR

SEQ ID NO: 19 - protein, 33 aa, Homo sapiens
MVIIVTVVSVLLSLFVTSVLLCFIFGQHLRQQR

SEQ ID NO: 20 - protein, 37 aa, Rattus sp.
PNKGSGTTSGTTRLLSGHTCFTLTGLLGTLVTMGLLT

SEQ ID NO: 21 - protein, 14 aa, Gallus gallus
MGSSKSKPKDPSQR

SEQ ID NO: 22 - protein, 11 aa, Rous sarcoma virus
MGQSLTTPLSL

SEQ ID NO: 23 - protein, 18 aa, Homo sapiens
SKDGKKKKKKSKTKCVIM

SEQ ID NO: 24 - protein, 11 aa, Rattus sp.
MVCCMRRTKQV

SEQ ID NO: 25 - protein, 14 aa, Mus musculus
CMSCKCVLKKKKKK

SEQ ID NO: 26 - protein, 26 aa, Homo sapiens
LLQRLFSRQDCCGNCSDSEEELPTRL

SEQ ID NO: 27 - protein, 20 aa, Rattus norvegicus
KQFRNCMLTSLCCGKNPLGD

SEQ ID NO: 28 - protein, 19 aa, Homo sapiens
LNPPDESGPGCMSCKCVLS

SEQ ID NO: 29 - protein, 19 aa, Mus musculus
  MOD_RES (11)..(11): palmitoyl group
LNPPDESGPGCMSCKCVLS

SEQ ID NO: 30 - protein, 5 aa, Artificial sequence
  lysosomal degradation sequence
KFERQ

SEQ ID NO: 31 - protein, 36 aa, Cricetulus griseus
MLIPIAGFFALAGLVLIVLIAYLIGRKRSHAGYQTI

SEQ ID NO: 32 - protein, 35 aa, Homo sapiens
LVPIAVGAALAGVLILVLLAYFIGLKHHHAGYEQF

SEQ ID NO: 33 - protein, 27 aa, Saccharomyces cerevisiae
MLRTSSLFTRRVQPSLFSRNILRLQST

SEQ ID NO: 34 - protein, 25 aa, Saccharomyces cerevisiae
MLSLRQSIRFFKPATRTLCSSRYLL

SEQ ID NO: 35 - protein, 64 aa, Saccharomyces cerevisiae
MFSMLSKRWAQRTLSKSFYSTATGAASKSGKLTQKLVTAGVAAAGITASTLLYADSLTAEAMTA

SEQ ID NO: 36 - protein, 41 aa, Saccharomyces cerevisiae
MKSFITRNKTAILATVAATGTAIGAYYYYNQLQQQQQRGKK

SEQ ID NO: 37 - protein, 4 aa, Homo sapiens
KDEL

SEQ ID NO: 38 - protein, 15 aa, unidentified adenovirus
LYLSRRSFIDEKKMP

SEQ ID NO: 39 - protein, 9 aa, Unknown
  cyclin B1 destruction box
RTALGDIGN

SEQ ID NO: 40 - protein, 20 aa, Unknown
  signal sequence from Interleukin-2
MYRMQLLSCIALSLALVTNS

SEQ ID NO: 41 - protein, 29 aa, Homo sapiens
MATGSRTSLLLAFGLLCLPWLQEGSAFPT

SEQ ID NO: 42 - protein, 27 aa, Homo sapiens
MALWMRLLPLLALLALWGPDPAAAFVN

SEQ ID NO: 43 - protein, 18 aa, Influenza virus
MKAKLLVLLYAFVAGDQI

SEQ ID NO: 44 - protein, 24 aa, Unknown
  signal sequence from Interleukin-4
MGLTSQLLPPLFFLLACAGNFVHG

SEQ ID NO: 45 - protein, 10 aa, Artificial sequence
  stability sequence
MGXXXXGGPP

SEQ ID NO: 46 - protein, 7 aa, Artificial sequence
  dimerization sequence
EFLIVKS

SEQ ID NO: 47 - protein, 9 aa, Artificial sequence
  dimerization sequence
EEFLIVKKS

SEQ ID NO: 48 - protein, 7 aa, Artificial sequence
  dimerization sequence
FESIKLV

SEQ ID NO: 49 - protein, 7 aa, Artificial sequence
  dimerization sequence
VSIKFEL

SEQ ID NO: 50 - protein, 10 aa, Artificial sequence
  dimerization sequence
EEEFLIVEEE

SEQ ID NO: 51 - protein, 10 aa, Artificial sequence
  dimerization sequence
KKKFLIVKKK

SEQ ID NO: 52 - protein, 5 aa, Artificial sequence
  linker consensus sequence
GSGGS

SEQ ID NO: 53 - protein, 4 aa, Artificial sequence
  linker consensus sequence
GGGS